(19) United States
(12) Patent Application Publication
(10) Pub. No.: US 2013/0324526 A1
(43) Pub. Date: Dec. 5, 2013

(54) [1,2,4] TRIAZOLO [4,3-B] PYRIDAZINE COMPOUNDS AS INHIBITORS OF THE C-MET TYROSINE KINASE

(71) Applicants: Feng He, Shanghai (CN); Haibing Deng, Shanghai (CN); Xingnian Fu, Lanzhou City (CN); Haibing Guo, Shanghai (CN); Yuan Mi, Shanghai (CN); Xiaoxia Yan, Shanghai (CN); Hongping Yu, Shanghai (CN); Ji Yue (Jeff) Zhang, Shanghai (CN)

(72) Inventors: Feng He, Shanghai (CN); Haibing Deng, Shanghai (CN); Xingnian Fu, Lanzhou City (CN); Haibing Guo, Shanghai (CN); Yuan Mi, Shanghai (CN); Xiaoxia Yan, Shanghai (CN); Hongping Yu, Shanghai (CN); Ji Yue (Jeff) Zhang, Shanghai (CN)

(73) Assignee: NOVARTIS AG, Basel (CH)

(21) Appl. No.: 13/984,429

(22) PCT Filed: Feb. 8, 2012
(86) PCT No.: PCT/EP2012/052147

§ 371 Date: Aug. 8, 2013

(30) Foreign Application Priority Data
Feb. 10, 2011 (CN) PCT/CN2011/070909
Dec. 14, 2011 (CN) PCT/CN2011/083960

(51) Int. Cl. C07D 471/04; A61K 31/5377; A61K 45/06; A61K 31/5025

(52) U.S. Cl. CPC C07D 471/04; A61K 31/5025; A61K 31/5377; A61K 45/06
USPC 514/233.2; 514/248; 544/236; 544/118

(57) ABSTRACT

The invention relates to compounds of formula (I) and salts thereof: wherein the substituents are as defined in the specification; a compound of formula (I) for use in the treatment of the human or animal body, in particular with regard to c-Met tyrosine kinase mediated diseases or conditions; the use of a compound of formula (I) for manufacturing a medicament for the treatment of such diseases; pharmaceutical compositions comprising a compound of the formula (I), optionally in the presence of a combination partner, and processes for the preparation of a compound of formula (I).

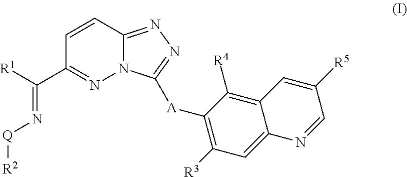

The invention relates to bicyclic compounds of formula (I) and salts thereof, the uses of such compounds to treat the human or animal body, in particular with regard to a proliferative disease, pharmaceutical compositions comprising such compounds, combinations comprising a compound of formula (I), and processes for the preparation of such compounds.

The Hepatocyte Growth Factor Receptor, herein referred to as c-Met, is a receptor tyrosine kinase that has been shown to be over-expressed and/or genetically altered in a variety of malignancies, specifically, gene amplification and a number of c-Met mutations are found in various solid tumors, see e.g. WO 2007/126799. Further, the receptor tyrosine kinase c-Met is involved in the processes of migration, invasion and morphogenesis that accompany embryogenesis and tissue regeneration. C-Met is also involved in the process of metastasis. Several lines of evidence have indicated that c-Met plays a role in tumor pathogenesis. Gain of function germ line mutations in c-Met is associated with development of hereditary papillary renal cell carcinoma (PRCC). Amplification or mutations in c-Met have also been reported in sporadic forms of PRCC, in head and neck squamous cell carcinoma, in gastric carcinoma, in pancreatic carcinoma and in lung cancer. Such alterations have been shown in selected instances to confer dependence of the tumor on c-Met and/or resistance to other targeted therapies. Elevated levels of c-Met, together with its unique ligand HGF/SF, are observed at high frequency in multiple clinically relevant tumors. A correlation between increased expression and disease progression, metastases and patient mortality has been reported in several cancers, including bladder, breast, squamous cell carcinoma and gastric carcinoma as well as leiomyosarcoma and glioblastoma.

WO 2008/008539 discloses certain fused heterocyclic derivatives which are useful in the treatment of HGF mediated diseases. WO 2007/075567, WO 2008/051805 and WO 2008/051808 disclose certain triazolopyridazine derivatives which are useful in the treatment of HGF mediated diseases. Furthermore, international patent applications PCT/EP2010/062057 and PCT/EP2010/061609 also disclose certain substituted triazolopyridazine derivatives with an oxime or hydrazone moiety which are useful in the treatment of c-Met mediated disorders.

It is an aim of the present invention to provide further compounds that modulate, and in particular inhibit, c-Met. It has now been found that the compounds of the formula (I) described herein are inhibitors of c-Met and have a number of therapeutic applications. For example, the compounds of formula (I) are suitable for use in the treatment of diseases dependent on c-Met activity, especially solid tumors or metastasis derived therefrom. Through the inhibition of c-Met, compounds of the invention also have utility as anti-inflammatory agents, for example for the treatment of an inflammatory condition which is due to an infection.

Preferably, the compounds of the invention are metabolically stable, are non-toxic and demonstrate few side-effects. In addition, preferred compounds of the invention exist in a physical form that is stable, non-hygroscopic and easily formulated. One aspect of the invention is directed to compounds of formula (I) having an activity that is at least similar, better superior to the activity of compounds of the prior art, or other similar compounds. Another aspect of the invention is directed to compounds of formula (I) having a good kinase selectivity. In particular, preferred compounds should have high affinity to the c-Met receptor and show functional antagonistic activity, while having little affinity for other kinase receptors or for targets known to be associated with adverse effects. In one aspect of the invention, preferred compounds demonstrate comparably low antagonistic activity against human PDE3 than related derivatives. Preferred compounds of the invention posses favourable pharmacokinetic properties, such as good in-vivo exposure and/or solubility and especially good metabolic stability, and/or do not form metabolites with unfavourable pharmacological properties.

The present invention relates to a compound of the formula (I)

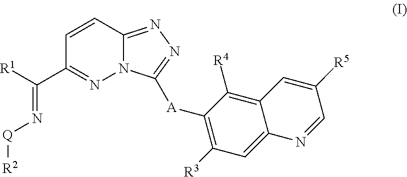

wherein
Q is O, NH or N(C₁-C₄)-alkyl,
A is a group selected from i or ii:

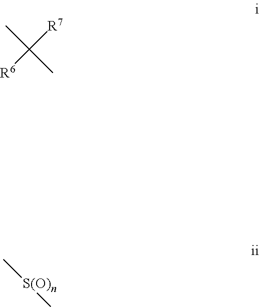

wherein
R⁶ is hydrogen, deuterium, OH, methyl or halo;
R⁷ is hydrogen, deuterium, halo, or (C₁-C₃)alkyl, wherein said (C₁-C₃)alkyl is optionally substituted by one or more substituents independently selected from OH and halo;
or R⁶ and R⁷, together with the carbon to which they are attached form cyclopropyl, wherein said cyclopropyl is optionally substituted by methyl;
n is 0, 1 or 2;
R¹ is hydrogen, NH₂, or (C₁-C₄)alkyl, wherein said (C₁-C₄)alkyl is optionally substituted by one or more substituents independently selected from OH, NH₃ and halo;
R² is
   Hydrogen,
   (C₁-C₄)alkyl, wherein said (C₁-C₄)alkyl is optionally substituted by one or more substituents independently selected from halo, hydroxy and methoxy, or
   —(C₀-C₂)alkyl(C₃-C₆)cycloalkyl;
R³ and R⁴ are independently selected from H and halo;
R⁵ is
   —(C₀-C₃)alkyl-heterocyclyl¹,
   —(C₀-C₃)alkyl-(C₃-C₈)cycloalkyl,
   —NR⁸R⁹, or
   (C₁-C₃)alkyl substituted by one or more OH [i.e. one, two or three OH] or by —N((C₁-C₃)alkyl)₂,
wherein R⁸ is hydrogen or (C₁-C₃)alkyl,
and R⁹ is (C₁-C₃)alkyl, (C₃-C₈)cycloalkyl, or heterocyclyl²,
   or a pharmaceutically acceptable salt thereof;
   with the proviso that the compound is not (E)-1-{3-[3-(4-Methyl-piperazin-1-yl)quinolin-6-ylmethyl]-[1,2,4]triazolo[4,3-b]pyridazin-6-yl}-ethanone O-(2-hydroxy-ethyl)-oxime.

In one embodiment, the present invention relates to a compound of the formula (I), wherein
Q is O, NH or N(C₁-C₄)-alkyl,
A is a group selected from i or ii:

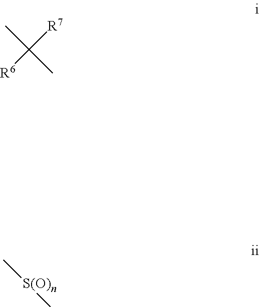

wherein
R⁶ is hydrogen, deuterium, OH, methyl or halo;
R⁷ is hydrogen, deuterium, halo, or (C₁-C₃)alkyl, wherein said (C₁-C₃)alkyl is optionally substituted by one or more substituents independently selected from OH and halo;
or R⁶ and R⁷, together with the carbon to which they are attached form cyclopropyl, wherein said cyclopropyl is optionally substituted by methyl;
n is 0, 1 or 2;
R¹ is hydrogen, NH₂, or (C₁-C₄)alkyl, wherein said (C₁-C₄)alkyl is optionally substituted by one or more substituents independently selected from OH, NH₃ and halo;
R² is
   hydrogen,
   (C₁-C₄)alkyl, wherein said (C₁-C₄)alkyl is optionally substituted by one or more substituents independently selected from halo, hydroxy and methoxy, or
   —(C₀-C₂)alkyl(C₃-C₆)cycloalkyl;
R³ and R⁴ are independently selected from H and halo;
R⁵ is
   —(C₀-C₃)alkyl-heterocyclyl¹,
   —(C₀-C₃)alkyl-(C₃-C₈)cycloalkyl, or
   (C₁-C₃)alkyl substituted by one or more OH [i.e. one, two or three OH],
or a pharmaceutically acceptable salt thereof;
with the proviso that the compound is not (E)-1-{3-[3-(4-Methyl-piperazin-1-yl)quinolin-6-ylmethyl]-[1,2,4]triazolo[4,3-b]pyridazin-6-yl}-ethanone O-(2-hydroxy-ethyl)-oxime.

The following general definitions shall apply in this specification, unless otherwise specified:

Unless specified otherwise, the term “compound of the invention”, or “compounds of the invention”, or “a compound of the present invention” or “compounds of the present invention” refer to compounds of Formula (I) and subformulae thereof, prodrugs thereof, salts of the compounds and/or prodrugs, hydrates or solvates of the compounds, salts and/or prodrugs, as well as all stereoisomers (including diastereoisomers and enantiomers), tautomers and isotopically labeled compounds (including deuterium substitutions), as well as inherently formed moieties (e.g., polymorphs, solvates and/or hydrates).

As used herein, the terms “including”, “containing” and “comprising” are used herein in their open, non-limiting sense.

Where the plural form (e.g. compounds, salts) is used, this includes the singular (e.g. a single compound, a single salt). “A compound” does not exclude that (e.g. in a pharmaceutical formulation) more than one compound of the formula (I) (or a salt thereof) is present.

“Halo” means fluoro, chloro, bromo or iodo. In a particular embodiment of the invention, halo is fluoro or chloro. In one embodiment, halo is fluoro.

Any non-cyclic carbon containing group or moiety with more than 1 carbon atom is straight-chain or branched.

“Alkyl” refers to a straight-chain or branched-chain alkyl group. For example, (C₁-C₄)alkyl includes methyl, ethyl, n- or iso-propyl, and n-, iso-, sec- or tert-butyl.

The term “cycloalkyl” refers to a saturated or unsaturated monocyclic hydrocarbon groups having 3, 4, 5, 6, 7 or 8 ring carbon atoms, in one embodiment from 3 up to and including 6 ring carbon atoms. Exemplary monocyclic hydrocarbon groups include, but are not limited to, cyclopropyl, cyclobutyl, cyclopentyl, cyclopentenyl, cyclohexyl and cyclohexenyl and the like.

The term “heterocyclyl¹” refers to a 4, 5, 6, 7 or 8 membered saturated, unsaturated or partially unsaturated mono- or bicyclic group comprising 1, 2 or 3 ring heteroatoms independently selected from N, O and S, wherein the total number of ring S atoms does not exceed 1, and the total number of ring O atoms does not exceed 1. Heterocyclyl¹ is optionally substituted by one or two substituents independently selected from —OH, —CONH₂, (C₁-C₃)alkyl, —N((C₁-C₃)alkyl)₂ and —NH₂, or in one embodiment (C₁-C₃)alkyl and —OH. Specific examples of heterocyclyl¹ include, but are not limited to, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, azetidinyl, tetrahydrofuryl, tetrahydrothiophenyl, 3,6-dihydro-2H-pyridinyl, 1,2,3,4-tetrahydropyridinyl, 1,2,5,6-tetrahydropyridinyl, pyrrolidinyl, thiazolidinyl, morpholinyl, thiomorpholinyl, piperidinyl, piperazinyl, quinuclidinyl, 2,5-diaza-bicyclo[2.2.1]heptyl, pyrrolyl, furanyl, thiophenyl, pyrazolyl, imidazolyl, isoxazolyl, oxazolyl, oxazolinyl, oxazolidinyl, isothiazolyl, thiazolyl, pyridinyl, pyridazinyl, pyrimidinyl, pyrazinyl, 3,4-dihydro-2H-pyranyl, 5,6-dihydro-2H-pyranyl, 2H-pyranyl, tetrahydropyranyl, dihydro-1 H-pyrrolyl, azepanyl, diazepanyl, oxazepanyl, and thiazepanyl. All these heterocyclyl¹ groups can be optionally substituted by one or two substituents independently selected from —OH, —CONH₂, (C₁-C₃)alkyl, —N((C₁-C₃)alkyl)₂ and —NH₂, preferably (C₁-C₃)alkyl, —OH and —NH₂, in particular one or two methyl groups or one —OH group. In one embodiment all these heterocyclyl¹ groups can be optionally substituted by (C₁-C₃)alkyl, —OH and —N(CH₃)₂, in particular one or two methyl groups or one dimethylamino or one —OH group.

In one embodiment, the term “heterocyclyl¹” refers to a 5, 6, 7 or 8 membered saturated, unsaturated or partially unsaturated mono- or bicyclic group comprising 1 or 2 ring heteroatoms independently selected from N, O and S, wherein the total number of ring S atoms does not exceed 1, and the total number of ring O atoms does not exceed 1. Heterocyclyl¹ is optionally substituted by one or two substituents independently selected from —OH, —CONH₂, (C₁-C₃)alkyl, —N((C₁-C₃)alkyl)₂ and —NH₂, in one embodiment one or two (C₁-C₃)alkyl groups or one —OH group. Examples of heterocyclyl¹ include, but are not limited to tetrahydrofuranyl, tetrahydrothiophenyl, 3,6-dihydro-2H-pyridinyl, 1,2,3,4-tetrahydropyridinyl, 1,2,5,6-tetrahydropyridinyl, pyrrolidinyl, thiazolidinyl, morpholinyl, piperidinyl, piperazinyl, quinuclidinyl, 2,5-diaza-bicyclo[2.2.1]heptyl, pyrrolyl, furanyl, thiophenyl, pyrazolyl, imidazolyl, isoxazolyl, oxazolyl, oxazolinyl, oxazolidinyl, isothiazolyl, thiazolyl, pyridinyl, pyridazinyl, pyrimidinyl, pyrazinyl, 3,4-dihydro-2H-pyranyl, 5,6-dihydro-2H-pyranyl, 2H-pyranyl, tetrahydropyranyl, dihydro-1 H-pyrrolyl, azepanyl, diazepanyl, oxazepanyl, and thiazepanyl. All these heterocyclyl¹ groups can be optionally substituted by one or two substituents independently selected from —OH, —CONH₂, (C₁-C₃)alkyl, —N((C₁-C₃)alkyl)₂ and —NH₂, in one embodiment (C₁-C₃)alkyl, —OH and —N((C₁-C₃)alkyl)₂, in particular by one or two methyl groups or —OH group. In one embodiment all these heterocyclyl¹ groups can be optionally substituted by (C₁-C₃)alkyl, —OH and —N(CH₃)₂, in particular one or two methyl groups or one dimethylamino or one —OH group.

In one embodiment heterocyclyl¹ includes tetrahydrofuranyl, tetrahydrothiophenyl, 3,6-dihydro-2H-pyridinyl, 1,2,3,4-tetrahydropyridinyl, 1,2,5,6-tetrahydropyridinyl, pyrrolidinyl, thiazolidinyl, morpholinyl, thiomorpholinyl, piperidinyl, quinuclidinyl, 2,5-diaza-bicyclo[2.2.1]heptyl, pyrrolyl, furanyl, thiophenyl, pyrazolyl, imidazolyl, isoxazolyl, oxazolyl, oxazolinyl, oxazolidinyl, isothiazolyl, thiazolyl, pyridinyl, pyridazinyl, pyrimidinyl, pyrazinyl, 3,4-dihydro-2H-pyranyl, 5,6-dihydro-2H-pyranyl, 2H-pyranyl, tetrahydropyranyl, dihydro-1H-pyrrolyl, azepanyl, diazepanyl, oxazepanyl, and thiazepanyl. All these heterocyclyl¹ groups can be optionally substituted by one or two substituents independently selected from —OH, —CONH₂, (C₁-C₃)alkyl, —N((C₁-C₃)alkyl)₂ and —NH₂, in one embodiment (C₁-C₃)alkyl, —OH and —N((C₁-C₃)alkyl)₂, in particular by one or two methyl groups or one —OH group. In one embodiment all these heterocyclyl¹ groups can be optionally substituted by (C₁-C₃)alkyl, —OH and —N(CH₃)₂, in particular one or two methyl groups or one dimethylamino or one —OH group.

In another embodiment heterocyclyl¹ includes 3,6-dihydro-2H-pyridin-1-yl, 1,2,3,4-tetrahydropyridin-1-yl, 1,2,5,6-tetrahydropyridin-1-yl, pyrrolidin-1-yl, thiazolidin-3-yl, morpholin-4-yl, thiomorpholin-4-yl, piperidin-1-yl, piperazin-1-yl, quinuclidin-1-yl, 2,5-diaza-bicyclo[2.2.1]hept-2-yl, pyrrol-1-yl, pyrazol-1-yl, imidazol-1-yl, H-isoxazol-2-yl, oxazol-3-yl, oxazolidin-3-yl, isothiazol-2-yl, thiazol-3-yl, pyridin-1-yl, pyridazin-1-yl, pyrimidin-1-yl, pyrazin-1-yl, dihydro-pyrrol-1-yl, azepan-1-yl, diazepan-1-yl, oxazepan-3-yl, and thiazepan-3-yl. All these heterocyclyl¹ groups can be optionally substituted by one or two substituents independently selected from —OH, —CONH₂, (C₁-C₃)alkyl, —N((C₁-C₃)alkyl)₂ and —NH₂, in one embodiment (C₁-C₃)alkyl, —OH and —N((C₁-C₃)alkyl)₂, in particular by one or two methyl groups or one —OH group. In one embodiment all these heterocyclyl¹ groups can be optionally substituted by (C₁-C₃)alkyl, —OH and —N(CH₃)₂, in particular one or two methyl groups or one dimethylamino or one —OH group. In one embodiment, heterocyclyl¹ includes the aforementioned groups except piperazin-1-yl.

In a further embodiment the term heterocyclyl¹ refers to morpholinyl, piperazinyl, piperidinyl, pyrrolidinyl, pyrazolyl, isoxazolyl, and 2,5-diaza-bicyclo[2.2.1]heptyl, all optionally substituted by one or two methyl groups or one —N(CH₃)₂ or one —OH group. In particular the term heterocyclyl¹ refers to morpholin-4-yl, piperazin-1-yl, piperidin-1-yl, pyrrolidin-1-yl, pyrazol-4-yl, isoxazol-4-yl, or 2,5-diaza-bicyclo[2.2.1]hept-2-yl, all optionally substituted by one or two methyl groups or one —N(CH₃)₂ or one —OH group.

In a further embodiment the term heterocyclyl¹ refers to morpholinyl, piperazinyl, piperidinyl, pyrazolyl, isoxazolyl, and 2,5-diaza-bicyclo[2.2.1]heptyl, all optionally substituted by one or two methyl groups or one —OH group. In particular the term heterocyclyl¹ refers to morpholin-4-yl, piperazin-1-yl, piperidin-1-yl, pyrazol-4-yl, isoxazol-4-yl, and 2,5-diaza-bicyclo[2.2.1]hept-2-yl, all optionally substituted by one or two methyl groups or one —OH group.

In a further embodiment, the term heterocyclyl¹ refers to morpholin-4-yl, 4-methylpiperazin-1-yl, piperidin-1-yl, 1-methyl-1H-pyrazol-4-yl, 3,5-dimethyl-isoxazol-4-yl, (1S,4S)-5-methyl-2,5-diaza-bicyclo[2.2.1]hept-2-yl, 3-dimethylamino-pyrrolidin-1-yl or 4-hydroxypiperidin-1-yl.

In a further embodiment, the term heterocyclyl¹ refers to morpholin-4-yl, 4-methylpiperazin-1-yl, piperidin-1-yl, 1-methyl-1H-pyrazol-4-yl, 3,5-dimethyl-isoxazol-4-yl, (1S,4S)-5-methyl-2,5-diaza-bicyclo[2.2.1]hept-2-yl, and 4-hydroxypiperidin-1-yl.

The term “heterocyclyl²” refers to a 5 or 6-membered saturated or partially unsaturated monocyclic group comprising 1 or 2 ring heteroatoms independently selected from N, O and S. Heterocyclyl² is optionally substituted by —OH or (C₁-C₃)alkyl. Specific examples of heterocyclyl² include, but are not limited to, tetrahydrofuranyl, tetrahydrothiophenyl, 3,6-dihydro-2H-pyridinyl, 1,2,3,4-tetrahydropyridinyl, 1,2,5,6-tetrahydropyridinyl, pyrrolidinyl, thiazolidinyl, morpholinyl, thiomorpholinyl, piperidinyl, oxazolinyl, oxazolidinyl, 3,4-dihydro-2H-pyranyl, 5,6-dihydro-2H-pyranyl, 2H-pyranyl, tetrahydropyranyl, and dihydro-1H-pyrrolyl. In one embodiment, heterocyclyl² includes piperidinyl and tetrahydropyranyl, in particular piperidin-4-yl and tetrahydropyran-4-yl. All these heterocyclyl² groups can be optionally substituted by one or two methyl groups.

In a further embodiment the term heterocyclyl² refers 1-methylpiperidin-4-yl or tetrahydro-2H-pyran-4-yl.

In one embodiment of the invention, A is i or ii′:

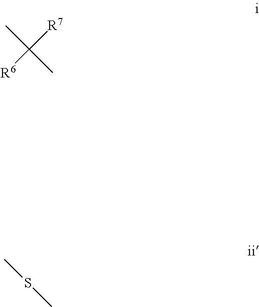

In one embodiment of the invention, A is i:

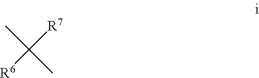

In another embodiment of the invention, Q is —O—. In this embodiment, compounds of the invention are of formula (Ia)

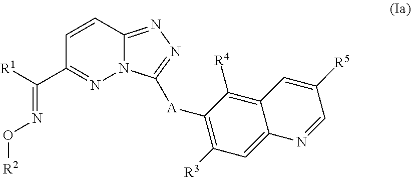

In one embodiment of the invention, R¹ is methyl.

In another embodiment of the invention, R² is hydrogen, or (C₁-C₂)alkyl, wherein said (C₁-C₂)alkyl is optionally substituted by one or more substituents independently selected from halo and hydroxy, or —(C₀-C₁)alkyl(C₃-C₆)cycloalkyl.

In one embodiment of the invention, R² is hydrogen, cyclopropylmethyl-, ethyl, methyl or 2-hydroxyethyl; in one embodiment R² is hydrogen.

In one embodiment of the invention, R³ and R⁴ are independently selected from hydrogen and fluoro; in one embodiment, R³ and R⁴ are either both hydrogen or R³ and R⁴ are both halogen, in particular fluoro.

In one embodiment of the invention, R⁵ is —(C₀-C₃)alkyl-heterocyclyl¹, —(C₀-C₃)alkyl-(C₃-C₈)cycloalkyl or (C₁-C₃)alkyl substituted by one or more OH [i.e. one, two or three OH] or by —N((C₁-C₃)alkyl)₂, or R⁵ is —NR⁸R⁹.

In a further embodiment of the invention, R⁵ is —(C₀-C₃)alkyl-heterocyclyl¹ or —(C₀-C₃)alkyl-(C₃-C₈)cycloalkyl.

In another embodiment R⁵ is —(C₁-C₃)alkyl-heterocyclyl¹ or —(C₀-C₃)alkyl-(C₃-C₈)cycloalkyl.

In an alternative embodiment R⁵ is —(C₀-C₁)alkyl-heterocyclyl¹ or —(C₀-C₁)alkyl-(C₃-C₆)cycloalkyl.

In a particular embodiment of the invention, R⁵ is —CH₂-heterocyclyl¹ or —(C₀-C₁)alkyl-(C₃-C₆)cycloalkyl, in particular —CH₂-heterocyclyl¹.

In all the above mentioned definitions for R⁵, the term heterocyclyl¹ in —(C₀-C₃)alkyl-heterocyclyl¹, —(C₁-C₃)alkyl-heterocyclyl¹, —(C₀-C₁)alkyl-heterocyclyl¹ or —CH₂-heterocyclyl¹, can have any of the aforementioned meanings of heterocyclyl¹.

In one embodiment, R⁵ is —(C₀-C₁)alkyl-heterocyclyl¹ or —(C₀-C₁)alkyl-(C₃-C₈)cycloalkyl, wherein heterocyclyl¹ is selected from tetrahydrofuranyl, tetrahydrothiophenyl, 3,6-dihydro-2H-pyridinyl, 1,2,3,4-tetrahydropyridinyl, 1,2,5,6-tetrahydropyridinyl, pyrrolidinyl, thiazolidinyl, morpholinyl, thiomorpholinyl, piperidinyl, piperazinyl, quinuclidinyl, 2,5-diaza-bicyclo[2.2.1]heptyl, pyrrolyl, furanyl, thiophenyl, pyrazolyl, imidazolyl, isoxazolyl, oxazolyl, oxazolinyl, oxazolidinyl, isothiazolyl, thiazolyl, pyridinyl, pyridazinyl, pyrimidinyl, pyrazinyl, 3,4-dihydro-2H-pyranyl, 5,6-dihydro-2H-pyranyl, 2H-pyranyl, tetrahydropyranyl, dihydro-1H-pyrrolyl, azepanyl, diazepanyl, oxazepanyl, and thiazepanyl, and wherein heterocyclyl¹ is optionally substituted by one or two methyl groups or one —N((C₁-C₃)alkyl)₂, —NH₂ or —OH group.

In a particular embodiment of the invention, R⁵ is morpholin-4-ylmethyl, methylpiperazin-1-ylmethyl, piperidin-1-ylmethyl, 1-methyl-1H-pyrazol-4-yl, morpholin-4-yl, (1S,4S)-5-methyl-2,5-diaza-bicyclo[2.2.1]hept-2-yl, dimethylamino-pyrrolidin-1-yl and 4-hydroxypiperidin-1-yl.

In a particular embodiment thereof, R⁵ is morpholin-4-ylmethyl, 4-methylpiperazin-1-ylmethyl, piperidin-1-ylmethyl, 1-methyl-1H-pyrazol-4-yl, morpholin-4-yl, (1S,4S)-5-methyl-2,5-diaza-bicyclo[2.2.1]hept-2-yl, and 4-hydroxypiperidin-1-yl.

In an alternative embodiment, R⁵ is —NR⁸R⁹, wherein R⁸ is hydrogen or (C₁-C₃)alkyl, and R⁹ is (C₁-C₃)alkyl, (C₃-C₈)cycloalkyl, or heterocyclyl² as defined herein.

In one embodiment thereof, R⁸ is hydrogen or methyl, in particular hydrogen, and R⁹ is cyclohexyl or heterocyclyl², in particular heterocyclyl², optionally substituted by methyl. In one embodiment thereof, heterocyclyl² is piperidin-4-yl or tetrahydropyran-4-yl.

In a particular embodiment, R⁵ is tetrahydro-pyran-4-ylamino- or 1-methyl-piperidin-4-ylamino-.

In another embodiment of the invention, R⁶ is hydrogen, deuterium, OH or halo, particularly hydrogen, deuterium or halo, and in another embodiment, R⁶ is hydrogen.

In another embodiment of the invention, R⁷ is hydrogen, deuterium, halo, or methyl, wherein said methyl is optionally substituted by one or more substituents independently selected from OH and halo. In another embodiment of the invention R⁷ is hydrogen, deuterium, halo, or methyl. In one embodiment, R⁷ is hydrogen or methyl, in particular hydrogen.

In a further embodiment of the invention, R⁶ and R⁷, together with the carbon to which they are attached form cyclopropyl, wherein said cyclopropyl is optionally substituted by methyl. In one embodiment, R⁶ and R⁷, together with the carbon to which they are attached form cyclopropyl.

In one embodiment of the invention, R⁶ and R⁷ are both hydrogen.

In an embodiment, where A is i, and R⁶ and R⁷ are not both hydrogen, the compound of formula (I) contains an asymmetric carbon atom at A. Included within the scope of the invention is a compound of formula (I) containing the (R), or the (S) enantiomer of A, or a mixture thereof. In another embodiment of the invention there is provided a compound of formula (I) containing the (S) enantiomer of Ai, or a mixture including the (S) enantiomer as a major component.

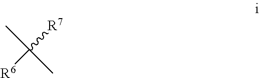

In another embodiment of the invention n is 0.

In a further embodiment the invention provides a compound of formula (I)

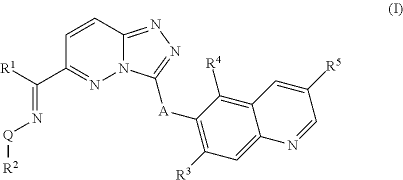

wherein
Q is O or NH,
A is a group selected from i or ii′:

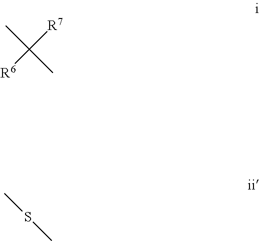

wherein
R⁶ is hydrogen;
R⁷ is hydrogen or methyl;
or R⁶ and R⁷, together with the carbon to which they are attached form cyclopropyl;
R¹ is methyl;
R² is
   hydrogen,
   (C₁-C₂)alkyl, wherein said (C₁-C₂)alkyl is optionally substituted by hydroxy, or
   —CH₂-cyclo(C₃-C₄)alkyl;
R³ and R⁴ are independently selected from hydrogen and fluoro;
R⁵ is
   heterocyclyl¹,
   —CH₂-heterocyclyl¹,
   —(C₀-C₁)alkyl-(C₃-C₆)cycloalkyl,
   —NR⁸R⁹, or
   (C₁-C₃)alkyl substituted by one or more OH [i.e. one, two or three OH] or by —N((C₁-C₃)alkyl)₂,
      wherein
heterocyclyl¹ is morpholin-4-yl, piperazin-1-yl, piperidin-1-yl, 1H-pyrazol-4-yl, isoxazol-4-yl, 2,5-diaza-bicyclo[2.2.1]hept-2-yl, pyrrolidin-1-yl, and wherein heterocyclyl¹ is optionally substituted by one or two methyl groups or one —NH₂ [or one —N(CH₃)₂] or one —OH group,
R⁸ is hydrogen or (C₁-C₃)alkyl,
and R⁹ is (C₁-C₃)alkyl, (C₃-C₆)cycloalkyl, or heterocyclyl², wherein heterocyclyl² is piperidin-4-yl or tetrahydropyran-4-yl, optionally substituted by methyl,
   or a pharmaceutically acceptable salt thereof;
   with the proviso that the compound is not (E)-1-{3-[3-(4-Methyl-piperazin-1-yl)quinolin-6-ylmethyl]-[1,2,4]triazolo[4,3-b]pyridazin-6-yl}-ethanone O-(2-hydroxy-ethyl)-oxime.

In one embodiment thereof, R⁵ is
heterocyclyl¹,
—CH₂-heterocyclyl¹,
—(C₀-C₁)alkyl-(C₃-C₆)cycloalkyl,
—(C₁-C₃)alkyl substituted by one or more OH [i.e. one, two or three OH],
   wherein
heterocyclyl¹ is morpholin-4-yl, piperidin-1-yl, 1H-pyrazol-4-yl, isoxazol-4-yl, 2,5-diaza-bicyclo[2.2.1]hept-2-yl, or pyrrolidin-1-yl, and wherein heterocyclyl¹ is optionally substituted by one or two methyl groups or one —NH₂ [or one —N(CH₃)₂] or one —OH group.

In another embodiment there is provided a compound of formula (I), wherein

### Q is —O—,

R¹ is methyl,
R² is hydrogen,

### A is —CH₂— or —S—,

R³ and R⁴ are independently selected from hydrogen and fluoro,
R⁵ is —(C₀-C₁)alkyl-heterocyclyl¹, wherein heterocyclyl¹ is selected from morpholinyl, piperidinyl, piperazinyl, pyrazolyl, isoxazolyl, 2,5-diaza-bicyclo[2.2.1]heptyl, and pyrrolidinyl, and wherein heterocyclyl¹ is optionally substituted by one or two methyl groups or one —NH₂ [or one —N(CH₃)₂] or one —OH group,
or a pharmaceutically acceptable salt thereof,
with the proviso that the compound is not (E)-1-{3-[3-(4-Methyl-piperazin-1-yl)quinolin-6-ylmethyl]-[1,2,4]triazolo[4,3-b]pyridazin-6-yl}ethanone O-(2-hydroxy-ethyl)-oxime,

In one embodiment thereof, R⁵ is —(C₀-C₁)alkyl-heterocyclyl¹, wherein heterocyclyl¹ is selected from morpholinyl, piperidinyl, pyrazolyl, isoxazolyl, 2,5-diaza-bicyclo[2.2.1]heptyl, and pyrrolidinyl, and wherein heterocyclyl¹ is optionally substituted by one or two methyl groups or one —NH₂ [or one —N(CH₃)₂] or one —OH group.

In an alternative embodiment thereof, R⁵ is —CH₂-heterocyclyl¹, wherein heterocyclyl¹ is selected from morpholinyl, piperidinyl, pyrazolyl, isoxazolyl, 2,5-diaza-bicyclo[2.2.1]heptyl, and pyrrolidinyl, and wherein heterocyclyl¹ is optionally substituted by one or two methyl groups or one one —NH₂ [or one —N(CH₃)₂] or one —OH group.

In an alternative embodiment thereof, R⁵ is —CH₂-heterocyclyl¹, wherein heterocyclyl¹ is selected from morpholin-4-yl, 4-methylpiperazin-1-yl, piperidin-1-yl, 1-methyl-1H-pyrazol-4-yl, 3,5-dimethyl-isoxazol-4-yl, (1S,4S)-5-methyl-2,5-diaza-bicyclo[2.2.1]hept-2-yl, 4-hydroxypiperidin-1-yl, and 3-dimethylamino-pyrrolidin-1-yl, in particular from morpholin-4-yl, 4-methylpiperazin-1-yl and piperidin-1-yl.

In a further embodiment thereof, R⁵ is selected from morpholin-4-ylmethyl, 4-methylpiperazin-1-ylmethyl, piperidin-1-ylmethyl, 1-methyl-1H-pyrazol-4-yl, morpholin-4-yl, 3,5-dimethyl-isoxazol-4-yl, (1S,4S)-5-methyl-2,5-diaza-bicyclo[2.2.1]hept-2-yl, 4-hydroxypiperidin-1-yl, and 3-amino-pyrrolidin-1-yl.

In a further embodiment thereof, R⁵ is selected from morpholin-4-ylmethyl, 4-methylpiperazin-1-ylmethyl, piperidin-1-ylmethyl, 1-methyl-1H-pyrazol-4-yl, morpholin-4-yl, 3,5-dimethyl-isoxazol-4-yl, (1S,4S)-5-methyl-2,5-diaza-bicyclo[2.2.1]hept-2-yl, 4-hydroxypiperidin-1-yl, and 3-dimethylamino-pyrrolidin-1-yl.

Various embodiments of the invention are described herein. It will be recognized that features specified in each embodiment may be combined with other specified features to provide further embodiments.

In a particular embodiment, the invention provides one or more individual compounds as those listed in the Examples section below, or a pharmaceutically acceptable salt or solvate thereof.

In another embodiment the invention provides a compound of the formula (I), which is selected from the following compounds:
No. 1 1-(3-{1-[3-(1-Methyl-1H-pyrazol-4-yl)quinolin-6-yl]-ethyl}-[1,2,4]triazolo[4,3-b]pyridazin-6-yl)-ethanone oxime
No. 2 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-ethyl-oxime
No. 3 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-methyl-oxime
No. 4 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-cyclopropylmethyl-oxime
No. 5 (E)-1-(3-{1-[3-(1-Methyl-1H-pyrazol-4-yl)quinolin-6-yl]-ethyl}-[1,2,4]triazolo[4,3-b]pyridazin-6-yl)-ethylidene]-hydrazine
No. 6 (E)-1-{3-[3-(1-Methyl-1H-pyrazol-4-yl)quinolin-6-ylmethyl]-[1,2,4]triazolo[4,3-b]pyridazin-6-yl}-ethanone O-methyl-oxime
No. 7 (E)-1-{3-[3-(1-Methyl-1H-pyrazol-4-yl)quinolin-6-ylmethyl]-[1,2,4]triazolo[4,3-b]pyridazin-6-yl}-ethanone O-cyclopropylmethyl-oxime
No. 8 (E)-1-{3-[3-(1-Methyl-1H-pyrazol-4-yl)quinolin-6-ylmethyl]-[1,2,4]triazolo[4,3-b]pyridazin-6-yl}-ethanone O-ethyl-oxime
No. 9 (E)-1-{3-[1-(3-(Morpholin-4-yl-methyl)quinolin-6-yl)-ethyl]-[1,2,4]triazolo[4,3-b]pyridazin-6-yl}-ethanone O-cyclopropylmethyl-oxime
No. 10 (E)-1-[3-(3-(Morpholin-4-yl)quinolin-6-ylsulfanyl)-[1,2,4]triazolo[4,3-b]pyridazin-6-yl]-ethanone oxime
No. 11 (E)-1-[3-((5,7-Difluoro-3-morpholin-4-yl-quinolin-6-yl)methyl)-[1,2,4]triazolo[4,3-b]pyridazin-6-yl]-ethanone oxime
No. 12 (E)-1-(3-((3-(1-Methyl-1H-pyrazol-4-yl)quinolin-6-yl)methyl)-[1,2,4]triazolo[4,3-b]pyridazin-6-yl)-ethanone oxime
No. 13 (E)-1-(3-((3-Morpholin-4-yl-quinolin-6-yl)methyl)-[1,2,4]triazolo[4,3-b]pyridazin-6-yl)-ethanone oxime

No. 14 (E)-1-(3-((3-(4-Methylpiperazin-1-yl)quinolin-6-yl)methyl)-[1,2,4]triazolo[4,3-b]pyridazin-6-yl)-ethanone oxime
No. 15 (E)-1-(3-((3-Morpholin-4-yl-methyl-quinolin-6-yl)methyl)-[1,2,4]triazolo[4,3-b]pyridazin-6-yl)-ethanone oxime
No. 16 (E)-1-(3-((3-(4-Methylpiperazin-1-yl-methyl)quinolin-6-yl)methyl)-[1,2,4]triazolo[4,3-b]pyridazin-6-yl)-ethanone oxime
No. 17 (E)-1-(3-((5,7-Difluoro-3-((morpholin-4-yl)-methyl)quinolin-6-yl)methyl)-[1,2,4]triazolo[4,3-b]pyridazin-6-yl)-ethanone oxime
No. 18 (E)-1-(3-((3-(Piperidin-1-ylmethyl)quinolin-6-yl)methyl)-[1,2,4]triazolo[4,3-b]pyridazin-6-yl)-ethanone oxime
No. 19 (E)-1-(3-((3-((1S,4S)-5-Methyl-2,5-diazabicyclo[2.2.1]heptan-2-yl)quinolin-6-yl)methyl)-[1,2,4]triazolo[4,3-b]pyridazin-6-yl)-ethanone oxime
No. 20 (E)-1-(3-((3-(4-Hydroxypiperidin-1-yl)quinolin-6-yl)methyl)-[1,2,4]triazolo[4,3-b]pyridazin-6-yl)-ethanone oxime
No. 21 (E)-1-(3-(1-(5,7-Difluoro-3-(1-methyl-1H-pyrazol-4-yl)quinolin-6-yl)ethyl)-[1,2,4]triazolo[4,3-b]pyridazin-6-yl)-ethanone oxime
No. 22 (E)-1-(3-((5,7-Difluoro-3-(1-methyl-1H-pyrazol-4-yl)quinolin-6-yl)methyl)-[1,2,4]triazolo[4,3-b]pyridazin-6-yl)-ethanone oxime
No. 23 (E)-1-(3-((3-(3,5-Dimethylisoxazol-4-yl)-5,7-difluoroquinolin-6-yl)methyl)-[1,2,4]triazolo[4,3-b]pyridazin-6-yl)-ethanone oxime
No. 24 (E)-1-(3-(1-(5,7-Difluoro-3-(2-hydroxypropan-2-yl)quinolin-6-yl)ethyl)-[1,2,4]triazolo[4,3-b]pyridazin-6-yl)-ethanone oxime
No. 25 (E)-1-(3-((3-Cyclohexyl-5,7-difluoroquinolin-6-yl)methyl)-[1,2,4]triazolo[4,3-b]pyridazin-6-yl)-ethanone O-2-hydroxyethyl oxime
No. 26 (E)-1-(3-((3-Cyclohexyl-5,7-difluoroquinolin-6-yl)methyl)-[1,2,4]triazolo[4,3-b]pyridazin-6-yl)-ethanone oxime
No. 27 (E)-1-(3-({1-[3-(4-Methyl-piperazin-1-yl)quinolin-6-yl]-cyclopropyl}-[1,2,4]triazolo[4,3-b]pyridazin-6-yl)-ethanone oxime
No. 28 (E)-1-(3-((3-(4-Methylpiperazin-1-yl)quinolin-6-yl)sulfanyl)-[1,2,4]triazolo[4,3-b]pyridazin-6-yl)-ethanone oxime
No. 29 (E)-1-(3-((3-(4-Hydroxypiperidin-1-yl)quinolin-6-yl)sulfanyl)-[1,2,4]triazolo[4,3-b]pyridazin-6-yl)-ethanone oxime
No. 30 (E)-1-(3-((3-((Tetrahydro-2H-pyran-4-yl)amino)quinolin-6-yl)sulfanyl)-[1,2,4]triazolo[4,3-b]pyridazin-6-yl)-ethanone oxime
No. 31 (E)-1-(3-((3-((Morpholin-4-yl)-methyl)quinolin-6-yl)sulfanyl)-[1,2,4]triazolo[4,3-b]pyridazin-6-yl)-ethanone oxime
No. 32 (E)-1-(3-((3-((Diethylamino)methyl)quinolin-6-yl)sulfanyl)-[1,2,4]triazolo[4,3-b]pyridazin-6-yl)ethanone oxime,
No. 33 (E)-1-(3-((3-(3-(Dimethylamino)pyrrolidin-1-yl)quinolin-6-yl)sulfanyl)-[1,2,4]triazolo[4,3-b]pyridazin-6-yl)ethanone oxime, and
No. 34 (E)-1-{3-[3-(Tetrahydro-pyran-4-ylamino)-quinolin-6-ylmethyl]-[1,2,4]triazolo[4,3-b]pyridazin-6-yl}-ethanone oxime.

In particular, the invention provides a compound of the formula (I), which is selected from compounds No. 1 to No. 27.

In a further embodiment, the invention provides a compound of the formula (I), which is selected from
No. 1 1-(3-{1-[3-(1-Methyl-1H-pyrazol-4-yl)quinolin-6-yl]-ethyl}-[1,2,4]triazolo[4,3-b]pyridazin-6-yl)-ethanone oxime
No. 2 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-methyl-oxime
No. 3 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-methyl-oxime
No. 4 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-cyclopropylmethyl-oxime
No. 5 (E)-1-(3-{1-[3-(1-Methyl-1H-pyrazol-4-yl)quinolin-6-yl]-ethyl}-[1,2,4]triazolo[4,3-b]pyridazin-6-yl)-ethylidene]-hydrazine
No. 6 (E)-1-{3-[3-(1-Methyl-1H-pyrazol-4-yl)quinolin-6-ylmethyl]-[1,2,4]triazolo[4,3-b]pyridazin-6-yl}-ethanone O-methyl-oxime
No. 7 (E)-1-{3-[3-(1-Methyl-1H-pyrazol-4-yl)quinolin-6-ylmethyl]-[1,2,4]triazolo[4,3-b]pyridazin-6-yl}-ethanone O-cyclopropylmethyl-oxime
No. 8 (E)-1-{3-[3-(1-Methyl-1H-pyrazol-4-yl)quinolin-6-ylmethyl]-[1,2,4]triazolo[4,3-b]pyridazin-6-yl}-ethanone O-ethyl-oxime
No. 9 (E)-1-{3-[1-(3-(Morpholin-4-yl-methyl)quinolin-6-yl)-ethyl]-[1,2,4]triazolo[4,3-b]pyridazin-6-yl}-ethanone O-cyclopropylmethyl-oxime
No. 10 (E)-1-[3-(3-(Morpholin-4-yl)quinolin-6-ylsulfanyl)-[1,2,4]triazolo[4,3-b]pyridazin-6-yl]-ethanone oxime
No. 11 (E)-1-[3-((5,7-Difluoro-3-(morpholin-4-yl)quinolin-6-yl)methyl)-[1,2,4]triazolo[4,3-b]pyridazin-6-yl]-ethanone oxime
No. 12 (E)-1-(3-((3-(1-Methyl-1H-pyrazol-4-yl)quinolin-6-yl)methyl)-[1,2,4]triazolo[4,3-b]pyridazin-6-yl)-ethanone oxime
No. 13 (E)-1-(3-((3-Morpholin-4-yl-quinolin-6-yl)methyl)-[1,2,4]triazolo[4,3-b]pyridazin-6-yl)-ethanone oxime
No. 15 (E)-1-(3-((3-Morpholin-4-yl-methyl-quinolin-6-yl)methyl)-[1,2,4]triazolo[4,3-b]pyridazin-6-yl)-ethanone oxime
No. 16 (E)-1-(3-((3-(4-Methylpiperazin-1-yl-methyl)quinolin-6-yl)methyl)-[1,2,4]triazolo[4,3-b]pyridazin-6-yl)-ethanone oxime
No. 17 (E)-1-(3-((5,7-Difluoro-3-((morpholin-4-yl)-methyl)quinolin-6-yl)methyl)-[1,2,4]triazolo[4,3-b]pyridazin-6-yl)-ethanone oxime
No. 18 (E)-1-(3-((3-(Piperidin-1-ylmethyl)quinolin-6-yl)methyl)-[1,2,4]triazolo[4,3-b]pyridazin-6-yl)-ethanone oxime
No. 19 (E)-1-(3-((3-((1S,4S)-5-Methyl-2,5-diazabicyclo[2.2.1]heptan-2-yl)quinolin-6-yl)methyl)-[1,2,4]triazolo[4,3-b]pyridazin-6-yl)-ethanone oxime
No. 20 (E)-1-(3-((3-(4-Hydroxypiperidin-1-yl)quinolin-6-yl)methyl)-[1,2,4]triazolo[4,3-b]pyridazin-6-yl)-ethanone oxime
No. 21 (E)-1-(3-(1-(5,7-Difluoro-3-(1-methyl-1H-pyrazol-4-yl)quinolin-6-yl)ethyl)-[1,2,4]triazolo[4,3-b]pyridazin-6-yl)-ethanone oxime
No. 22 (E)-1-(3-((5,7-Difluoro-3-(1-methyl-1H-pyrazol-4-yl)quinolin-6-yl)methyl)-[1,2,4]triazolo[4,3-b]pyridazin-6-yl)-ethanone oxime
No. 23 (E)-1-(3-((3-(3,5-Dimethylisoxazol-4-yl)-5,7-difluoroquinolin-6-yl)methyl)-[1,2,4]triazolo[4,3-b]pyridazin-6-yl)-ethanone oxime
No. 24 (E)-1-(3-(1-(5,7-Difluoro-3-(2-hydroxypropan-2-yl)quinolin-6-yl)ethyl)-[1,2,4]triazolo[4,3-b]pyridazin-6-yl)-ethanone oxime
No. 25 (E)-1-(3-((3-Cyclohexyl-5,7-difluoroquinolin-6-yl)methyl)-[1,2,4]triazolo[4,3-b]pyridazin-6-yl)-ethanone O-2-hydroxyethyl oxime, and
No. 26 (E)-1-(3-((3-Cyclohexyl-5,7-difluoroquinolin-6-yl)methyl)-[1,2,4]triazolo[4,3-b]pyridazin-6-yl)-ethanone oxime.

In another embodiment, the invention relates to a compound which is selected from the group consisting of
No. 1 1-(3-{1-[3-(1-Methyl-1H-pyrazol-4-yl)quinolin-6-yl]-ethyl}-[1,2,4]triazolo[4,3-b]pyridazin-6-yl)-ethanone oxime
No. 2 1-(3-{1-[3-(1-Methyl-1H-pyrazol-4-yl)quinolin-6-yl]-ethyl}-[1,2,4]triazolo[4,3-b]pyridazin-6-yl)-ethanone O-ethyl-oxime
No. 3 1-(3-{1-[3-(1-Methyl-1H-pyrazol-4-yl)quinolin-6-yl]-ethyl}-[1,2,4]triazolo[4,3-b]pyridazin-6-yl)-ethanone O-methyl-oxime
No. 4 1-(3-{1-[3-(1-Methyl-1H-pyrazol-4-yl)quinolin-6-yl]-ethyl}-[1,2,4]triazolo[4,3-b]pyridazin-6-yl)-ethanone O-cyclopropylmethyl-oxime
No. 5 1-(3-{1-[3-(1-Methyl-1H-pyrazol-4-yl)quinolin-6-yl]-ethyl}-[1,2,4]triazolo[4,3-b]pyridazin-6-yl)-ethylidene]-hydrazine
No. 6 1-{3-[3-(1-Methyl-1H-pyrazol-4-yl)quinolin-6-ylmethyl]-[1,2,4]triazolo[4,3-b]pyridazin-6-yl}-ethanone O-methyl-oxime
No. 7 1-{3-[3-(1-Methyl-1H-pyrazol-4-yl)quinolin-6-ylmethyl]-[1,2,4]triazolo[4,3-b]pyridazin-6-yl}-ethanone O-cyclopropylmethyl-oxime
No. 8 1-{3-[3-(1-Methyl-1H-pyrazol-4-yl)quinolin-6-ylmethyl]-[1,2,4]triazolo[4,3-b]pyridazin-6-yl}-ethanone O-ethyl-oxime
No. 9 1-{3-[1-(3-(Morpholin-4-yl-methyl)quinolin-6-yl)-ethyl]-[1,2,4]triazolo[4,3-b]pyridazin-6-yl}-ethanone O-cyclopropylmethyl-oxime
No. 10 1-[3-(3-(Morpholin-4-yl)quinolin-6-ylsulfanyl)-[1,2,4]triazolo[4,3-b]pyridazin-6-yl]-ethanone oxime
No. 11 1-[3-((5,7-Difluoro-3-morpholin-4-yl-quinolin-6-yl)methyl)-[1,2,4]triazolo[4,3-b]pyridazin-6-yl]-ethanone oxime
No. 12 1-(3-((3-(1-Methyl-1H-pyrazol-4-yl)quinolin-6-yl)methyl)-[1,2,4]triazolo[4,3-b]pyridazin-6-yl)-ethanone oxime
No. 13 1-(3-((3-Morpholin-4-yl-quinolin-6-yl)methyl)-[1,2,4]triazolo[4,3-b]pyridazin-6-yl)-ethanone oxime
No. 14 1-(3-((3-(4-Methylpiperazin-1-yl)quinolin-6-yl)methyl)-[1,2,4]triazolo[4,3-b]pyridazin-6-yl)-ethanone oxime
No. 15 1-(3((3-Morpholin-4-yl-methyl-quinolin-6-yl)methyl)-[1,2,4]triazolo[4,3-b]pyridazin-6-yl)-ethanone oxime
No. 16 1-(3-((3-(4-Methylpiperazin-1-yl-methyl)quinolin-6-yl)methyl)-[1,2,4]triazolo[4,3-b]pyridazin-6-yl)-ethanone oxime
No. 17 1-(3-((5,7-Difluoro-3-((morpholin-4-yl)-methyl)quinolin-6-yl)methyl)-[1,2,4]triazolo[4,3-b]pyridazin-6-yl)-ethanone oxime
No. 18 1-(3-((3-(Piperidin-1-ylmethyl)quinolin-6-yl)methyl)-[1,2,4]triazolo[4,3-b]pyridazin-6-yl)-ethanone oxime
No. 19 1-(3-((3-(5-Methyl-2,5-diazabicyclo[2.2.1]heptan-2-yl)quinolin-6-yl)methyl)-[1,2,4]triazolo[4,3-b]pyridazin-6-yl)-ethanone oxime
No. 20 1-(3-((3-(4-Hydroxypiperidin-1-yl)quinolin-6-yl)methyl)-[1,2,4]triazolo[4,3-b]pyridazin-6-yl)-ethanone oxime
No. 21 1-(3-(1-(5,7-Difluoro-3-(1-methyl-1H-pyrazol-4-yl)quinolin-6-yl)ethyl)-[1,2,4]triazolo[4,3-b]pyridazin-6-yl)-ethanone oxime
No. 22 1-(3-((5,7-Difluoro-3-(1-methyl-1H-pyrazol-4-yl)quinolin-6-yl)methyl)-[1,2,4]triazolo[4,3-b]pyridazin-6-yl)-ethanone oxime
No. 23 1-(3-((3-(3,5-Dimethylisoxazol-4-yl)-5,7-difluoroquinolin-6-yl)methyl)-[1,2,4]triazolo[4,3-b]pyridazin-6-yl)-ethanone oxime
No. 24 1-(3-(1-(5,7-Difluoro-3-(2-hydroxypropan-2-yl)quinolin-6-yl)ethyl)-[1,2,4]triazolo[4,3-b]pyridazin-6-yl)-ethanone oxime
No. 25 1-(3-((3-Cyclohexyl-5,7-difluoroquinolin-6-yl)methyl)-[1,2,4]triazolo[4,3-b]pyridazin-6-yl)-ethanone O-2-hydroxyethyl oxime
No. 26 1-(3((3-Cyclohexyl-5,7-difluoroquinolin-6-yl)methyl)-[1,2,4]triazolo[4,3-b]pyridazin-6-yl)-ethanone oxime,
No. 27 1-(3-({1-[3-(4-Methyl-piperazin-1-yl)quinolin-6-yl]-cyclopropyl}-[1,2,4]triazolo[4,3-b]pyridazin-6-yl)-ethanone oxime,
No. 28 1-(3-((3-(4-Methylpiperazin-1-yl)quinolin-6-yl)sulfanyl)-[1,2,4]triazolo[4,3-b]pyridazin-6-yl)-ethanone oxime
No. 29 1-(3-((3-(4-Hydroxypiperidin-1-yl)quinolin-6-yl)sulfanyl)-[1,2,4]triazolo[4,3-b]pyridazin-6-yl)-ethanone oxime
No. 30 1-(3-((3-((Tetrahydro-2H-pyran-4-yl)amino)quinolin-6-yl)sulfanyl)-[1,2,4]triazolo[4,3-b]pyridazin-6-yl)-ethanone oxime
No. 31 1-(3((3-((Morpholin-4-yl)-methyl)quinolin-6-yl)sulfanyl)-[1,2,4]triazolo[4,3-b]pyridazin-6-yl)-ethanone oxime
No. 32 1-(3-((3-((Diethylamino)methyl)quinolin-6-yl)sulfanyl)-[1,2,4]triazolo[4,3-b]pyridazin-6-yl)ethanone oxime,
No. 33 1-(3-((3-(3-(Dimethylamino)pyrrolidin-1-yl)quinolin-6-yl)sulfanyl)-[1,2,4]triazolo[4,3-b]pyridazin-6-yl)ethanone oxime, and
No. 34 1-{3-[3-(Tetrahydro-pyran-4-ylamino)-quinolin-6-ylmethyl]-[1,2,4]triazolo[4,3-b]pyridazin-6-yl}-ethanone oxime.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a disubstituted cycloalkyl, the cycloalkyl substituent may have a cis- or trans-configuration. All tautomeric forms are also intended to be included.

Any asymmetric atom (e.g., carbon or the like) of the compound(s) of the present invention can be present in racemic or enantiomerically enriched, for example the (R)-, (S)- or (R,S)-configuration, such as for the asymmetric carbon atom which may be present within the A group (i) defined herein. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. In one embodiment, for the asymmetric A group (i) defined herein, the (S) enantiomer is in excess, in amounts as described above.

Substituents at atoms with unsaturated bonds may, if possible, be present in cis-(Z)- or trans-(E)-form. In one embodiment, the hydrazones of the present invention have the trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this invention any atom specifically designated as a deuterium (D) is meant to represent deuterium, for example in the ranges given above.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Pharmaceutically acceptable solvates include hydrates and other solvates wherein the solvent of crystallization may be isotopically substituted, e.g. D₂O, d₆-acetone, d₆-DMSO.

The compounds of the present invention, including salts, hydrates and solvates thereof, may inherently or by design form polymorphs.

The compounds of the invention therefore include compounds of formula I, as well as their pharmaceutically acceptable salts, polymorphs, solvates and isomers (including optical, geometric and tautomeric isomers) and isotopically-labelled compounds of formula I, as defined herein, as well as mixtures thereof.

In particular embodiments, which are selected independently, collectively or in any combination or sub-combination, the invention relates to a compound of the formula (I), in free base form or in acid addition salt form, wherein the substituents are as defined herein.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by c-Met or (ii) associated with c-Met activity, or (iii) characterized by activity (normal or abnormal) of c-Met; or (2) reducing or inhibiting the activity of c-Met; or (3) reducing or inhibiting the expression of c-Met. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of c-Met; or at least partially reducing or inhibiting the expression of c-Met.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

“Disease” as used herein includes a disorder or condition.

“C-Met tyrosine kinase mediated diseases” are especially such disorders that respond in a beneficial way (e.g. amelioration of one or more symptoms, delay of the onset of a disease, up to temporary or complete cure from a disease) to the inhibition of a protein tyrosine kinase, especially inhibition of a c-Met kinase. These disorders include proliferative diseases such as tumor diseases, in particular solid tumors and metastasis derived thereof, e.g. hereditary papillary renal cell carcinoma (PRCC), sporadic forms of PRCC, head and neck cancer, squamous cell carcinoma, gastric carcinoma, pancreatic carcinoma, lung cancer, bladder cancer, breast cancer, leiomyosarcoma, glioblastoma, melanoma, alveolar soft part sarcoma. These disorders further include inflammatory conditions, such as inflammatory conditions due to an infection.

“Combination” refers to either a fixed combination in one dosage unit form, or a kit of parts for the combined administration where a compound of the formula (I) and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients.

The term “fixed combination” means that the active ingredients, e.g. a compound of formula (I) and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The compounds of formula I in free form or in salt form, exhibit valuable pharmacological properties, c-Met kinase inhibiting properties, e.g. as indicated in in vitro and in vivo tests as provided herewithin and are therefore indicated for therapy.

In another embodiment of the invention, there is provided a method for treating a c-Met related disorder or condition by administering a compound of the present invention. The disorder or condition to be treated is preferably a proliferative disease such as a cancer or an inflammatory condition. Compounds of formula (I) are further useful for treating diseases associated with a c-Met-related condition.

A: Proliferative diseases: Compounds of formula (I) are particular useful for the treatment of one or more of the following proliferative diseases:

Compounds of formula (I) are useful in the treatment of cancer wherein the cancer is selected from the group consisting of brain cancer, stomach cancer, genital cancer, urinary cancer, prostate cancer, bladder cancer (superficial and muscle invasive), breast cancer, cervical cancer, colon cancer, colorectal cancer, glioma (including glioblastoma, anaplastic astrocytoma, oligoastrocytoma, oligodendroglioma), esophageal cancer, gastric cancer, gastrointestinal cancer, liver cancer, hepatocellular carcinoma (HCC) including childhood HCC, head and neck cancer (including head and neck squamous-cell carcinoma, nasopharyngeal carcinoma), Hurthle cell carcinoma, epithelial cancer, skin cancer, melanoma (including malignant melanoma), mesothelioma, lymphoma, myeloma (including multiple myeloma), leukemias, lung cancer (including non-small cell lung cancer (including all histological subtypes: adenocarcinoma, squamous cell carcinoma, bronchoalveolar carcinoma, large-cell carcinoma, and adenosquamous mixed type), small-cell lung cancer), ovarian cancer, pancreatic cancer, prostate cancer, kidney cancer (including but not limited to papillary renal cell carcinoma), intestine cancer, renal cell cancer (including hereditary and sporadic papillary renal cell cancer, Type I and Type II, and clear cell renal cell cancer); sarcomas, in particular osteosarcomas, clear cell sarcomas, and soft tissue sarcomas (including alveolar and embryonal rhabdomyosarcomas, alveolar soft part sarcomas); thyroid carcinoma (papillary and other subtypes).

Compounds of formula (I) are useful in the treatment of cancer wherein the cancer is stomach, colon, liver, genital, urinary, melanoma, or prostate. In a particular embodiment, the cancer is liver or esophageal.

Compounds of formula (I) are useful in the treatment of colon cancer, including metastases, e.g. in the liver, and of non-small-cell lung carcinoma.

Compounds of formula (I) may also be used in the treatment of hereditary papillary renal carcinoma (Schmidt, L. et al. Nat. Genet. 16, 68-73, 1997) and other proliferative diseases in which c-M ET is overexpressed or constitutively activated by mutations (Jeffers and Vande Woude. Oncogene 18, 5120-5125, 1999; and reference cited therein) or chromosomal rearrange-ments (e.g. TPR-MET; Cooper et al. Nature 311, 29-33, 1984; Park. et al. Cell 45, 895-904, 1986).

Compounds of formula (I) are further useful in the treatment of additional cancers and conditions as provided herein or known in the art.

B: Inflammatory conditions: Compounds of formula (I) are particular suitable for the treatment of one or more inflammatory conditions.

In a further embodiment, the inflammatory condition is due to an infection. In one embodiment, the method of treatment would be to block pathogen infection. In a particular embodiment, the infection is a bacterial infection, e.g., a Listeria infection. See, e.g., Shen et al. Cell 103: 501-10, (2000) whereby a bacterial surface protein activates c-Met kinase through binding to the extracellular domain of the receptor, thereby mimicking the effect of the cognate ligand HGF/SF.

Compounds of formula (I) are further useful in the treatment of additional inflammatory disorders and conditions as provided herein or known in the art.

C: Combination therapy: In certain embodiments, any of the above methods involve further administering a chemotherapeutic agent.

In a related embodiment, the chemotherapeutic agent is an anti-cancer agent. Specific combinations are provided throughout the application.

In a further related embodiment, any of the above methods involve further administering a pathway specific inhibitor. The pathway specific inhibitor may be a chemotherapeutic agent or may be a biologic agent, e.g., such as antibodies. Pathway specific inhibitors include, but are not limited to, inhibitors of EGFR, Her-2, Her-3, VEGFR, Ron, IGF-IR, PI-3K, mTOR, Raf.

In a further related embodiment to several of the above methods, following administration to the subject, these methods can further involve observing amelioration or retardation of development or metastasis of the cancer.

Thus, in one embodiment, the invention relates to a method of treating a c-Met related disorder or condition which involves administering to a subject in need thereof an effective amount of a compound of formula (I).

In a further embodiment, the invention relates to a compound of formula (I) or a pharmaceutically acceptable salt thereof, for use as a medicament, in particular for the treatment of one or more c-Met tyrosine kinase mediated diseases.

In a further embodiment, the invention relates to the use of a compound of formula (I) or a pharmaceutically acceptable salt thereof, in the manufacture of a medicament for the treatment of one or more c-Met tyrosine kinase mediated diseases.

In a further embodiment, the invention relates to a method for the treatment of a disease or disorder which responds to an inhibition of c-Met tyrosine kinase, which comprises administering a compound of formula (I) or a pharmaceutically acceptable salt thereof, especially in a quantity effective against said disease, to a warm-blooded animal requiring such treatment.

In a further embodiment, the invention relates to a pharmaceutical composition comprising a compound of formula (I) as active ingredient in association with at least one pharmaceutical carrier or diluent.

In a further embodiment, the invention relates to a pharmaceutical composition comprising: (a) an effective amount of compound of formula (I) and/or pharmaceutically acceptable salts thereof, and/or pharmaceutically active metabolites thereof; and (b) one or more pharmaceutically acceptable excipients and/or diluents.

In a further embodiment, the invention relates to a pharmaceutical composition for treatment of a disease, e.g. of solid or liquid tumours in warm-blooded animals, including humans, comprising a dose effective in the treatment of said disease of a compound of the formula (I) as described above or a pharmaceutically acceptable salt of such a compound together with a pharmaceutically acceptable carrier (=carrier material).

In another embodiment of the invention, there is provided a pharmaceutical preparation (composition), comprising a compound of formula (I) as defined herein, or a pharmaceutically acceptable salt of such a compound, or a hydrate or solvate thereof, and at least one pharmaceutically acceptable carrier and/or diluents and optionally one or more further therapeutic agents.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated, the route of administration, the route of administration; the renal and hepatic function of the patient; and the particular compound employed. A physician, clinician or veterinarian of ordinary skill can readily determine the effective amount of each of the active ingredients necessary to prevent, treat or inhibit the progress of the disorder or disease. Optimal precision in achieving concentration of drug within the range that yields efficacy without toxicity requires a regimen based on the kinetics of the drug's availability to target sites. This involves a consideration of the distribution, equilibrium, and elimination of a drug.

The dose of a compound of the formula (I) or a pharmaceutically acceptable salt thereof to be administered to warm-blooded animals, for example humans of approximately 70 kg body weight, is preferably from approximately 3 mg to approximately 5 g, more preferably from approximately 10 mg to approximately 1.5 g per person per day, divided preferably into 1 to 3 single doses which may, for example, be of the same size. Usually, children receive half of the adult dose.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

The compound of the present invention may be administered either simultaneously with, or before or after, one or more other therapeutic agent. The compound of the present invention may be administered separately, by the same or different route of administration, or together in the same pharmaceutical composition as the other agents.

In one embodiment, the invention provides a product comprising a compound of formula (I) and at least one other therapeutic agent as a combined preparation for simultaneous, separate or sequential use in therapy. In one embodiment, the therapy is the treatment of a disease or condition mediated by c-Met tyrosine kinase. Products provided as a combined preparation include a composition comprising the compound of formula (I) and the other therapeutic agent(s) together in the same pharmaceutical composition, or the compound of formula (I) and the other therapeutic agent(s) in separate form, e.g. in the form of a kit.

In one embodiment, the invention provides a pharmaceutical composition comprising a compound of formula (I) and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

A compound of formula (I) can besides or in addition be administered especially for tumor therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Thus, a compound of the formula (I) may be used in combination with other anti-proliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibittors; mTOR inhibitors; antineoplastic antimetabolites; platin compounds; compounds targeting/decreasing a protein or lipid kinase activity; anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors; kinesin spindle protein inhibitors; MEK inhibitors; leucovorin; EDG binders; antileukemia compounds; ribonucleotide reductase inhibittors; S-adenosylmethionine decarboxylase inhibitors; angiostatic steroids; corticosteroids; other chemotherapeutic compounds (as defined below); photosensitizing compounds.

Further, alternatively or in addition they may be used in combination with other tumor treatment approaches, including surgery, ionizing radiation, photodynamic therapy, implants, e.g. with corticosteroids, hormones, or they may be used as radiosensitizers.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating compound” as used herein includes, but is not limited to, cyclophos-phamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA). Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl}-2E-2-propenamide, or a pharmaceutically acceptable salt thereof, especially the lactate salt.

The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity”; or a “protein or lipid phosphatase activity”; or “further anti-angiogenic compounds” as used herein includes, but is not limited to, c-Met tyrosine kinase and/or serine and/or threonine kinase inhibitors or lipid kinase inhibitors, e.g.,

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the fibroblast growth factor-receptors (FGFR);
c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
d) compounds targeting, decreasing or inhibiting the activity of the Trk receptor tyrosine kinase family, or ephrin kinase family inhibitors;
e) compounds targeting, decreasing or inhibiting the activity of the Axl receptor tyrosine kinase family;
f) compounds targeting, decreasing or inhibiting the activity of the Ret receptor tyrosine kinase;
g) compounds targeting, decreasing or inhibiting the activity of the Kit/SCFR receptor tyrosine kinase, e.g. imatinib;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN₁₀₇); PD180970; AG957; NSC 680410; PD173955 from ParkeDavis; or dasatinib (BMS-354825)
j) compounds targeting, decreasing or inhibiting the activity of members of the protein kinase C(PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a PI3K inhibitor) or AT7519 (CDK inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr <1500) compound, or a pharmaceutically acceptable salt thereof, especially a compound selected from the benzylidenemalonitrile class or the 5-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
l) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™) cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF;
n) compounds targeting, decreasing or inhibiting the activity of the Ron receptor tyrosine kinase.

Further anti-angiogenic compounds include compounds having another mechanism for their activity, e.g. unrelated to protein or lipid kinase inhibition e.g. thalidomide (THALOMID) and TNP-470.

The term “Compounds which target, decrease or inhibit the activity of a protein or lipid phosphatase” includes, but is not limited to inhibitors of phosphatase 1, phosphatase 2A, or CDC₂₅, e.g. okadaic acid or a derivative thereof.

The term “Compounds which induce cell differentiation processes” includes, but is not limited to e.g. retinoic acid, α- γ- or δ-tocopherol or α- γ- or δ-tocotrienol.

The term “cyclooxygenase inhibitor” as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “mTOR inhibitors” relates to compounds which inhibit the mammalian target of rapamycin (mTOR) and which possess antiproliferative activity such as sirolimus (Rapamune®), everolimus (Certican™), CCl-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™)and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

The term “Compounds which target, decrease or inhibit the activity of FMS-like tyrosine kinase receptors (Flt-3R)” are especially compounds, proteins or antibodies which inhibit members of the Flt-3R receptor kinase family, e.g. PKC₄₁₂, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17AAG, 17-DMAG), a geldanamycin derivative; other geldanamycin related compounds; radicicol and HDAC inhibitors; IPI-504, CNF1010, CNF2024, CNF1010 from Conforma Therapeutics; temozolomide (TEMODAL®), AUY922 from Novartis.

The term “antiproliferative antibodies” as used herein includes, but is not limited to, trastuzumab (Herceptin™), Trastuzumab-DM1, erbitux, bevacizumab (Avastin™) rituximab (Rituxan®), PRO64553 (anti-CD40) and 2C₄ Antibody. By antibodies is meant e.g. intact monoclonal antibodies, polyclonal antibodies, multispecific antibodies formed from at least 2 intact antibodies, and antibodies fragments so long as they exhibit the desired biological activity.

The term “antileukemic compounds” includes, for example, Ara-C, a pyrimidine analog, which is the 2″-alpha-hydroxy ribose (arabinoside) derivative of deoxycytidine. Also included is the purine analog of hypoxanthine, 6-mercaptopurine (6-MP) and fludarabine phosphate. For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC_{412.}

“Somatostatin receptor antagonists” as used herein refers to compounds which target, treat or inhibit the somatostatin receptor such as octreotide, and SOM230.

“Tumor cell damaging approaches” refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term “EDG binders” as used herein refers a class of immunosuppressants that modulates lymphocyte recirculation, such as Fingolimod (FTY720).

The term “kinesin spindle protein inhibitors” is known in the field and includes SB715992 or SB743921 from GlaxoSmithKline, pentamidine/chlorpromazine from CombinatoRx;

The term “MEK inhibitors” is known in the field and includes ARRY142886 from Array PioPharma, AZD6244 from AstraZeneca, PD181461 from Pfizer, leucovorin.

The term “ribonucleotide reductase inhibitors” includes, but is not limited to to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF/VEGFR disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Nat/Aced Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Bevacizumab (Avastin™)

“Photodynamic therapy” as used herein refers to therapy which uses certain chemicals known as photosensitizing compounds to treat or prevent cancers. Examples of photodynamic therapy include treatment with compounds, such as e.g. VISUDYNE and porfimer sodium.

“Angiostatic steroids” as used herein refers to compounds which block or inhibit angiogenesis, such as, e.g., anecortave, triamcinolone. hydrocortisone, 11-α-epihydrocotisol, cortexolone, 17α-hydroxyprogesterone, corticosterone, desoxycorticosterone, testosterone, estrone and dexamethasone.

“Corticosteroids” as used herein includes, but is not limited to compounds, such as e.g. fluocinolone, dexamethasone; in particular in the form of implants.

“Other chemotherapeutic compounds” include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

A compound of formula (I) may also be used in combination with one or more further drug substances selected from the group of anti-inflammatory drug substances; antihistamine drug substances; bronchodilatatory drug substances, NSAID; antagonists of chemokine receptors.

The compounds of the invention are also useful as co-therapeutic compounds for use in combination with such further drug substances, particularly in the treatment of inflammatory diseases such as those mentioned hereinbefore, for example as potentiators of therapeutic activity of such drugs or as a means of reducing required dosaging or potential side effects of such drugs. A compound of the invention may be mixed with such other drug substance in a fixed pharmaceutical composition or it may be administered separately (i.e. before, simultaneously with or after the other drug substance). Accordingly, the invention includes a combination of a compound of formula (I) with one or more further drug substance selected from the group of anti-inflammatory drug substances; antihistamine drug substances; bronchodilatatory drug substances, NSAID antagonists of chemokine receptors; said compound of the formula (I) and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445, WO 03/072592, non-steroidal glucocorticoid receptor agonists such as those described in WO 00/00531, WO 02/10143, WO 03/082280, WO 03/082787, WO 03/104195, WO 04/005229; LTB4 antagonists such LY293111, CGS025019C, CP-195543, SC-53228, BIIL 284, ONO 4057, SB 209247 and those described in U.S. Pat. No. 5,451,700; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID(TM) CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2a agonists such as those disclosed in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; A2b antagonists such as those described in WO 02/42298; and beta-2 adrenoceptor agonists such as albuterol (salbutamol), metaproterenol, terbutaline, salmeterol fenoterol, procaterol, and especially, formoterol and pharmaceutically acceptable salts thereof, and compounds (in free or salt or solvate form) of formula I of WO 0075114, which document is incorporated herein by reference, preferably compounds of the Examples thereof, especially a compound of formula

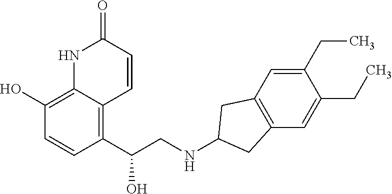 [SMILES: CCc1cc2c(cc1CC)CC(NC[C@H](O)c1ccc(O)c3[nH]c(=O)ccc13)C2]

and pharmaceutically acceptable salts thereof, as well as compounds (in free or salt or solvate form) of formula I of WO 04/16601, and also compounds of WO 04/033412. Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, WO 03/33495 and WO 04/018422.

Suitable chemokine receptors include, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-amin-ium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in WO 03/099807, WO 04/026841 and JP 2004107299.

Therapeutic agents for possible combination are especially one or more antiproliferative, cytostatic or cytotoxic compounds, for example one or several agents selected from the group which includes, but is not limited to, an inhibitor of polyamine biosynthesis, an inhibitor of a protein kinase, especially of a serine/threonine protein kinase, such as protein kinase C, or of a tyrosine protein kinase, such as the EGF receptor tyrosine kinase, e.g. Iressa®, the VEGF receptor tyrosine kinase, e.g. PTK787 or Avastin®, an antibody against the ligand VEGF, or the PDGF receptor tyrosine kinase, e.g. STI571 (Glivec®), PI3K (such as BEZ₂₃₅ from Novartis) and mToR inhibitors, such as rapamycin, RAD001, a cytokine, a negative growth regulator, such as TGF-11 or IFN-11, an aromatase inhibitor, e.g. letrozole (Femara®) or anastrozole, an inhibitor of the interaction of an SH2 domain with a phosphorylated protein, antiestrogens, topoisomerase I inhibitors, such as irinotecan, topoisomerase II inhibitors, microtubule active agents, e.g. paclitaxel or an epothilone, alkylating agents, antiproliferative antimetabolites, such as gemcitabine or capecitabine, platin compounds, such as carboplatin or cis-platin, bisphosphonates, e.g. AREDIA® or ZOMETA®, and monoclonal antibodies, e.g. against HER2, such as trastuzumab.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

The above-mentioned compounds, which can be used in combination with a compound of the formula (I), can be prepared and administered as described in the art, such as in the documents cited above.

Accordingly, the invention provides the use of a compound of formula (I) for treating a disease or condition mediated by c-Met tyrosine kinase, wherein the medicament is prepared for administration with another therapeutic agent as exemplified above. The invention also provides the use of another therapeutic agent for treating a disease or condition mediated by c-Met tyrosine kinase, wherein the medicament is administered with a compound of formula (I).

The invention also provides a compound of formula (I) for use in a method of treating a disease or condition mediated by c-Met tyrosine kinase, wherein the compound of formula (I) is prepared for administration with another therapeutic agent.

The invention also provides another therapeutic agent for use in a method of treating a disease or condition mediated by c-Met tyrosine kinase, wherein the other therapeutic agent is prepared for administration with a compound of formula (I). The invention also provides a compound of formula (I) for use in a method of treating a disease or condition mediated by c-Met tyrosine kinase, wherein the compound of formula (I) is administered with another therapeutic agent. The invention also provides another therapeutic agent for use in a method of treating a disease or condition mediated by c-Met tyrosine kinase, wherein the other therapeutic agent is administered with a compound of formula (I).

Thus, the invention relates in a further embodiment to a combination, particularly a pharmaceutical composition) comprising a therapeutically effective amount of a compound of formula (I) in free form or in pharmaceutically acceptable salt form and a second therapeutically active agent, for simultaneous or sequential administration. The additional therapeutic agent is preferably selected from the group consisting of an anti-cancer agent; an anti-inflammatory agent.

The invention further relates to a method for the treatment of a disease or mediated by c-Met tyrosine kinase, especially a proliferative disorder or disease, in particular a cancer, said method comprises administration of an effective amount of a combination of pharmaceutical agents which comprise: (a) a compound of formula (I); and (b) one or more pharmaceutically active agents, to a subject in need thereof, especially a human.

The invention further relates to the use of a combination of pharmaceutical agents which comprise: (a) a compound of formula (I); and (b) one or more pharmaceutically active agents for the treatment of a disease or disorder mediated by c-Met tyrosine kinase, especially a proliferative disorder or disease, in particular a cancer.

The invention further relates to the use of a combination of pharmaceutical agents which comprise: (a) a compound of formula (I); and (b) one or more pharmaceutically active agents, for the manufacture of a medicament for the treatment of a disease or disorder mediated by c-Met tyrosine kinase, especially a proliferative disorder or disease, in particular a cancer.

The invention further relates to pharmaceutical compositions comprising (a) a compound of formula (I) and (b) a pharmaceutically active agent; and (c) a pharmaceutically acceptable carrier; wherein at least one pharmaceutically active agent is an anti-cancer therapeutic.

The present invention further relates to a commercial package or product comprising: (a) a compound of formula (I); and (b) a pharmaceutical formulation of a pharmaceutically active agent for simultaneous, concurrent, separate or sequential use; wherein at least one pharmaceutically active agent is an anti-cancer therapeutic.

The invention also provides the use of a compound of formula (I) for treating a disease or condition mediated by c-Met tyrosine kinase, wherein the patient has previously (e.g. within 24 hours) been treated with another therapeutic agent. The invention also provides the use of another therapeutic agent for treating a disease or condition mediated by c-Met tyrosine kinase, wherein the patient has previously (e.g. within 24 hours) been treated with a compound of formula (I).

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

In another embodiment of the invention, there is provided a method of manufacturing a compound of formula (I) and intermediates thereof. A compound of the formula (I) may be prepared by processes that, though not applied hitherto for the new compounds of the present invention where they thus form new processes, are known per se. The schemes provide a general overview of synthetic strategies to obtain a compound of formula (I). All methods described can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Thus, the invention relates in a further aspect to a manufacturing process (a method for manufacturing) a compound of formula (I) comprising at least one reaction step as disclosed herein, and intermediates thereof.

The compounds of the present invention may be prepared by use of known chemical reactions and procedures. Nevertheless, the following general preparative methods are presented to aid the reader in synthesizing the compounds of formula (I), with specific details provided below in the experimental section to illustrate working examples. All variable groups of these methods are as described in the generic description if they are not specifically defined below.

It is recognized that compounds of the invention with each claimed optional functional group may not be prepared by each of the below-listed methods. Within the scope of each method, optional substituents may appear on reagents or intermediates which may act as protecting or otherwise non-participating groups. Utilizing methods well known to those skilled in the art, these groups are introduced and/or removed during the course of the synthetic schemes which provide the compounds of the present invention.

Typically, the compounds of formula (I) can be prepared according to the Schemes provided infra

Scheme 1 provides details for a synthetic strategy to obtain preferred compounds of formula (I) starting from (II).

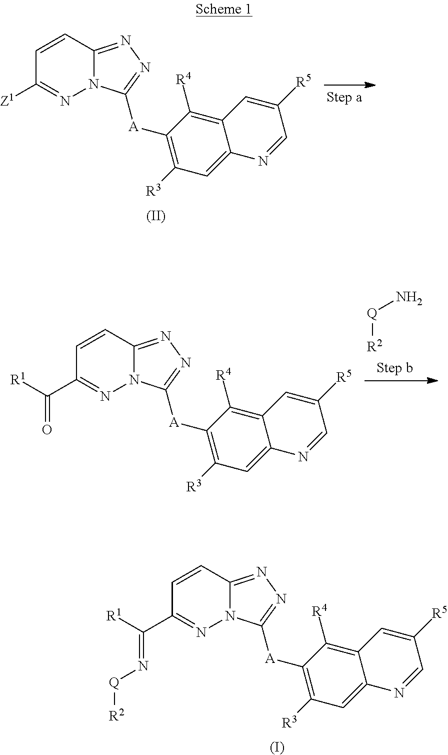

Z₁ is selected from Cl, Br and I or from COOH and COOMe.

Depending on the nature of Z₁, the reaction(s) carried out in Step a will be different.

Scheme 2 provides details for a synthetic strategy to obtain preferred compounds of formula (IB) through (IIB) starting from (III B) or (IVB).

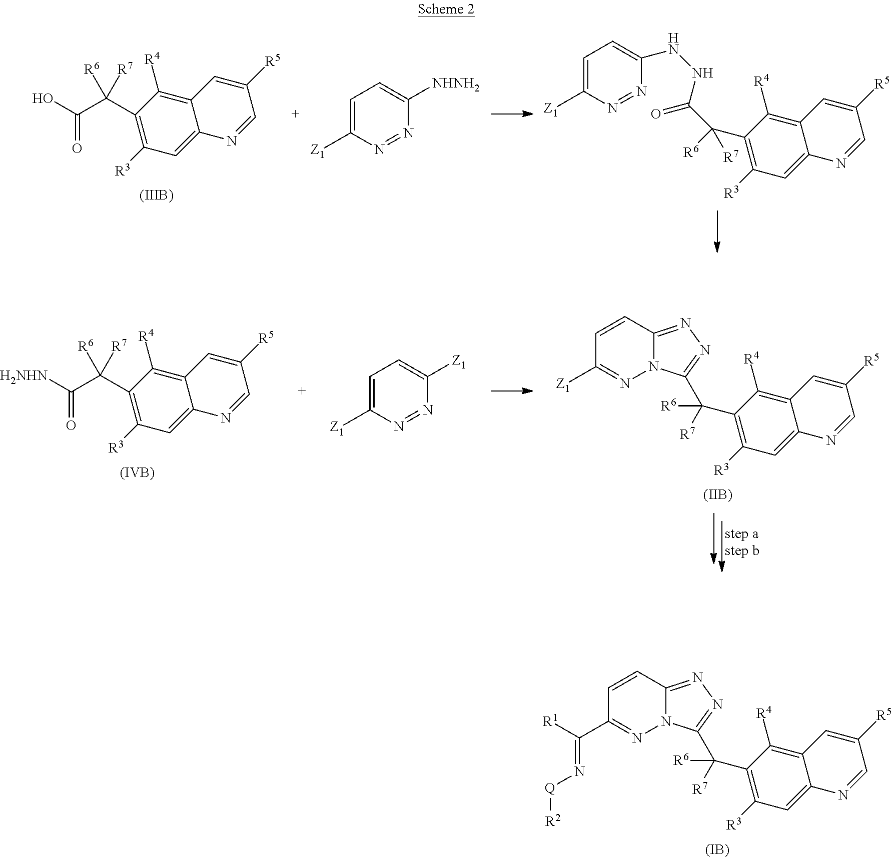

Z₁ is selected from Cl, Br and I.

Scheme 3 provides details for an alternative synthetic strategy to obtain preferred compounds of formula (IB) through (VB) starting from (VIC).

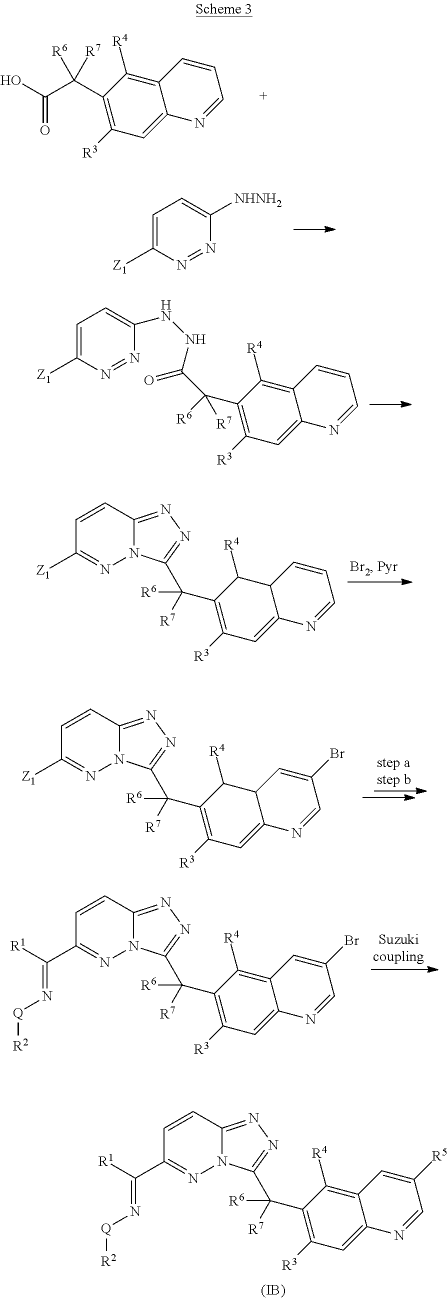

Z₁ is selected from COOH and COOMe.

Scheme 4 provides details for a synthetic strategy to obtain preferred compounds of formula (IC) through (IIC) starting from (VC).

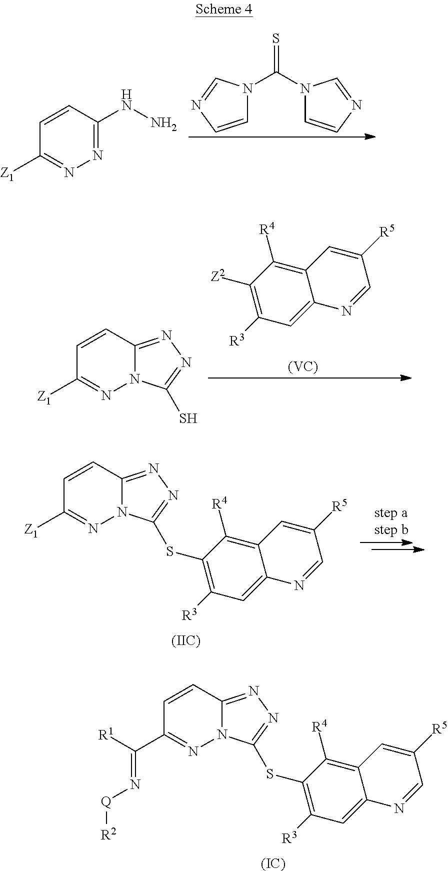

Z₁ is independently selected from COOH and COOMe.
Z₂ is independently selected from Cl, Br, I and OTf.

Oxidation of the —S— linker thereby using methods well known to the skilled person delivers SO/SO₂ linkers.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the invention and intermediates can also be converted into each other according to methods generally known to those skilled in the art.

The following examples illustrate the invention without limiting the scope thereof. In the examples provided, temperatures are measured in degrees Celsius. Unless otherwise indicated, the reactions take place at room temperature (rt). Further, if not indicated otherwise, the analytical and preparative HPLC conditions are as follows:

### Method A:

The flow is 1.2 mL/min of methanol and water (with 0.5% acetic acid)

0-2.0 min: 10% to 90% of methanol
2.0-3.0 min: 90% of methanol
Column: GP C18 3 μm 4.6×30 mm from Sepax.
Oven temperature: 30° C.

### Method B

The flow is 1.5 mL/min of methanol and water (with 0.5% formic acid)

0-2.0 min: 10% to 90% of methanol
2.0-3.0 min 90% of methanol
Column: GP C18 3 μm 4.6×30 mm from Sepax.
Oven temperature: 30° C.

### Method C

SFC equipment: Thar SFC Prep 80
The flow is 45 g/min of Methanol/CO₂ 75/25

### Column: CHIRALPAK AD-H, 2.0×25 cm

Wave length: UV 254 nm
Oven temperature: 35° C.

In the following examples, the abbreviations given below are used:
AcOH acetic acid
aq. aqueous
atm. atmosphere
BINAP 2,2′-bis-diphenylphosphinyl-[1,1′]binaphthalenyl
Bn benzyl
Boc tert-butoxycarbonyl
DCC dicyclohexylcarbodiimide
DCM dichloromethane
DME 1,2-dimethoxyethane
Et₂O diethyl ether
EtOAc or EA ethyl acetate
EtOH ethanol
DME dimethyl ethylene glycol
DMF N,N-dimethylformamide
DMSO dimethyl sulfoxide
eq. equivalent(s)
h hour(s)
HATU 2-(1H-7-Azabenzotriazol-1-yl)-1,1,3,3-tetramethyluronium hexafluorophosphate
HPLC High Performance Liquid Chromatography
HV high vacuum
IBX 2-iodoxybenzoic acid
Isolute Isolute® HM-N by International Solvent Technology Ltd., U.K. LAH lithium aluminium hydride
LCMS liquid chromatography coupled with mass spectrometry
LDA lithium diisopropylamide
mL milliliter(s)
min minute(s)
MPLC Medium Pressure Liquid Chormatography
MS-ES electrospray mass spectrometry
MW microwave
NBS N-bromosuccinimide
n-BuLi n-butyllithium
NMP N-methylpyrrolidinone
PdCl₂(dppf) 1,1-bis(diphenylphosphino)ferrocenedichloropalladium (II)
Pd₂(dba)₃ tris(dibenzylideneacetone)dipalladium (0)
PdCl₂(Ph₃)₂ dichlorobis(triphenylphosphine)palladium (II)
R_{f} ratio of fronts in TLC
rt room temperature
TBAF tetrabutylammonium fluoride
TBME methyl tert-butyl ether
TFA trifluoroacetic acid
THF tetrahydrofuran
TLC thin layer chromatography
t_{R} retention time
UV Ultraviolet

### Syntheses of Intermediates

### Intermediate A

### 3-(1-Methyl-1H-pyrazol-4-yl)quinolin-6-yl]-acetic acid ethyl ester

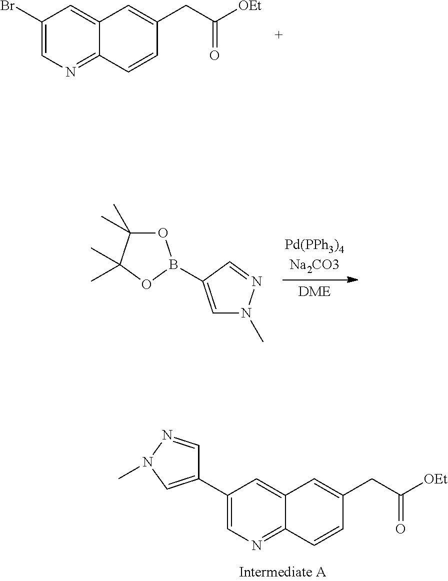

A mixture of methyl 2-(3-bromoquinolin-6-yl)acetate (800 mg, 2.86 mmol), 1-methyl-4-(4,4,5,5-tetramethyl-1,3,2-dioxaborolan-2-yl)-1H-pyrazole (713 mg, 3.43 mmol), Pd(PPh₃)₄ (330 mg, 0.286 mmol) and aqueous Na₂CO₃ solution (2 M, 2 mL) in DME (5 mL) was bubbled with argon for about 5 min, then sealed and irritated under microwave at 120° C. for 45 min. The reaction mixture was diluted with water. The aqueous phase was extracted with DCM:i-PrOH (v/v=3:1) three times. The combined organic phase was dried over anhydrous MgSO₄, filtered and concentrated. The crude product was purified by column chromatography on silica gel (eluting with 7% MeOH in DCM) to give the title compound as yellow solid (1.3 g, 89%). LCMS (method A): [M+H]⁺=296, t_{R}=2.20 min.

### Intermediate A1

### 2-[3-(1-Methyl-1H-pyrazol-4-yl)quinolin-6-yl]-propionic acid ethyl ester

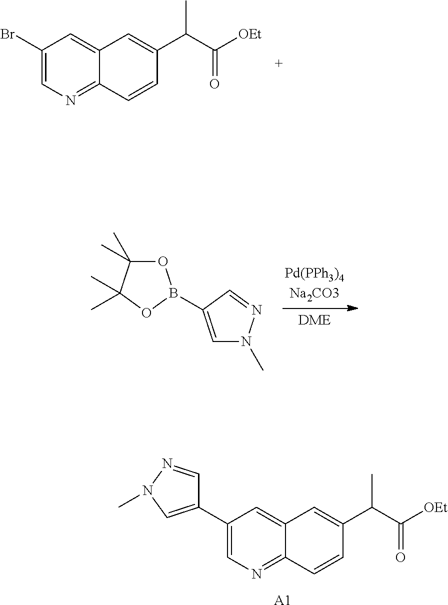

A mixture of ethyl 2-(3-bromoquinolin-6-yl)propanoate (10 g, 32.4 mmol), 1-methyl-4-(4,4,5,5-tetramethyl-1,3,2-dioxaborolan-2-yl)-1H-pyrazole (7.43 g, 35.7 mmol), Pd(PPh₃)₄ (3.75 g, 3.24 mmol) and Na₂CO₃ (2M, 10 mL) was bubbled with argon for 10 min. Then the mixture was heated at reflux for 6 h. Diluted with water, the water phase was extracted with DCM:i-PrOH (3:1) three times. The combined organic phase was dried over anhydrous MgSO₄, filtered and concentrated. The crude product was purified by chromatography (eluting with 7% MeOH in DCM) to give the title compound as yellow solid (9.5 g, yield 95%). LCMS (method A): [M+H]⁺=310, t_{R}=2.28 min.

### Intermediate B

### Methyl 2-(3-(morpholin-4-yl-methyl)quinolin-6-yl)acetate

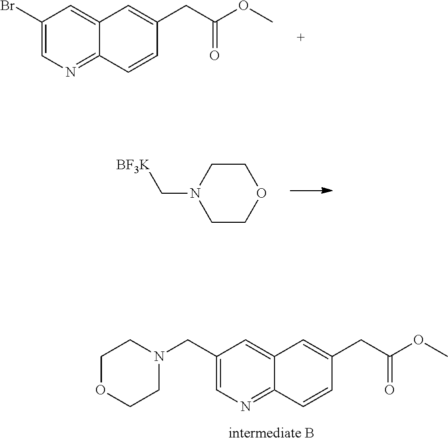

A solution of methyl 2-(3-bromoquinolin-6-yl)acetate (560 mg, 2.0 mmol) and potassium trifluoro(morpholin-4-yl-methyl)borate (418 mg, 2.019 mmol) in THF/H₂O (v/v, 4/1, 8 mL) was purged with argon for 3 min, followed by addition of Pd(OAc)₂ (13.34 mg, 0.06 mmol), Xphos (57.2 mg, 0.12 mmol) and Cs₂CO₃ (426 mg, 4.43 mmol) sequentially. The mixture was purged with argon for another half min. The reaction mixture was stirred at 80° C. for 18 h under argon. Then the reaction mixture was cooled to rt, water was added and the product was then extracted with EtOAc. The organic layers were combined, dried over Na₂SO₄ and concentrated under reduced pressure. The residue was purified by column chromatography (EtOAc) to afford 320 mg (53%) of the title compound. ¹H-NMR (400 MHz, CDCl₃) δ ppm 8.89 (s, 1H), 8.08 (d, 2H), 7.72 (s, 1H), 7.64 (d, 1H), 3.83 (s, 2H), 3.74 (s_{b}, 9H), 2.53 (s_{b}, 4H). LCMS (method A): [MH]⁺=301, t_{R}=1.017 min.

### Intermediates B1, B2 and B3

### [3-(4-Methyl-piperazin-1-ylmethyl)-quinolin-6-yl]-acetic acid methyl ester (B1)

### (5,7-Difluoro-3-morpholin-4-ylmethyl-quinolin-6-yl)-acetic acid methyl ester (B2)

### (3-Piperidin-1-ylmethyl-quinolin-6-yl)-acetic acid methyl ester (B3)

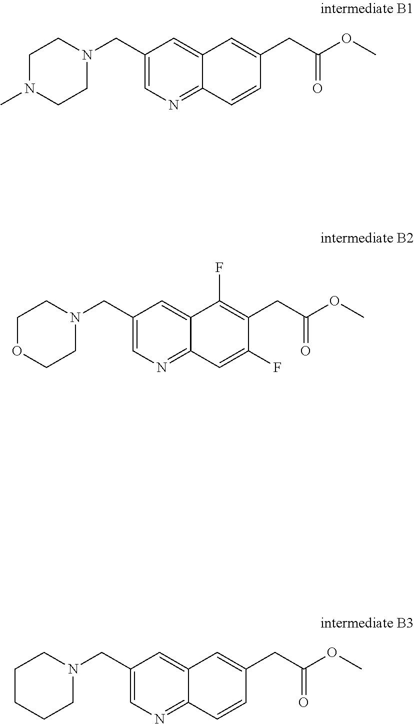

Intermediates B1, B2 and B3 were prepared using the same procedure as described for intermediate B.

### Intermediate C

### 2-(3-((1S,4S)-5-methyl-2,5-diazabicyclo[2.2.1]heptan-2-yl)quinolin-6-yl)acetic acid methyl ester

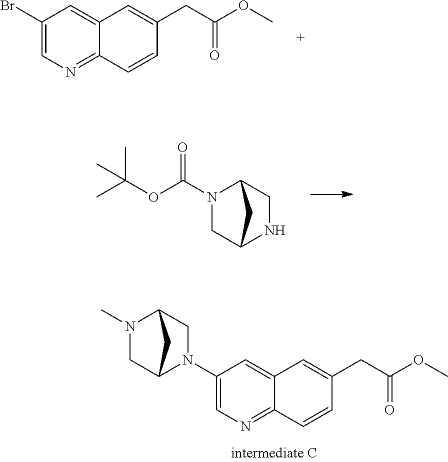

A solution of methyl 2-(3-bromoquinolin-6-yl)acetate (1.12 g, 4.0 mmol) and (1S,4S)-tert-butyl 2,5-diazabicyclo[2.2.1]heptane-2-carboxylate (1.189 g, 6.0 mmol) in toluene (15 mL) was purged with argon for 3 min, followed by addition of Pd₂(dba)₃ (366 mg, 0.4 mmol), Xantphos (463 mg, 0.8 mmol) and t-BuONa (576 mg, 6.0 mmol) sequentially. The mixture was purged with argon for another half min. The reaction mixture was stirred at 115° C. for 2 h under argon. Then the reaction mixture was cooled to rt and the solvent was removed under reduced pressure. The residue was diluted with methanol (20 mL), then SOCl₂ (2 mL) was added dropwised and the reaction mixture was stirred at rt overnight. Solvent methanol was removed and the residue was dissolved in water, neutralized with NaHCO₃ aqueous solution, extracted with dichloromethane. The organic layers were combined, dried over Na₂SO₄ and concentrated under reduced pressure. The crude product was dissolved in formic acid (10 mL) and formaldehyde (37% aqueous solution, 1 mL) and the reaction mixture was stirred at reflux for 1 h. Then the reaction mixture was cooled to rt and the solvent was removed under reduced pressure. The residue was dissolved in water, neutralized with NaHCO₃ aqueous solution, extracted with dichloromethane. The organic layers were combined, dried over Na₂SO₄ and concentrated under reduced pressure. The residue was purified by column chromatography on silica gel (20% methanol in dichloromethane) to afford 368 mg (30%) of the title compound. ¹H-NMR (400 MHz, CDCl₃) δ ppm 8.50 (s, 1H), 7.91 (d, 1H), 7.51 (s, 1H), 7.33 (d, 1H), 6.97 (s, 1H), 4.40 (s, 1H), 3.77 (s, 2H), 3.72 (s, 3H), 3.62 (s, 1H), 3.56-3.41 (m, 2H), 3.06 (d, 1H), 2.72 (d, 1H), 2.42 (s, 3H), 2.11 (d, 1H), 2.00 (d, 1H). LCMS (method A): [MH]⁺=312, t_{R}=1.218 min.

### Intermediates C1, C2, C3 and C4

### (3-Morpholin-4-yl-quinolin-6-yl)-acetic acid methyl ester (C1)

### [3-(4-Methyl-piperazin-1-yl)-quinolin-6-yl]-acetic acid methyl ester (C2)

### 1-[3-(4-Methyl-piperazin-1-yl)-quinolin-6-yl]-cyclopropanecarboxylic acid methyl ester (C3)

### [3-(Tetrahydro-pyran-4-ylamino)-quinolin-6-yl]-acetic acid methyl ester (C4)

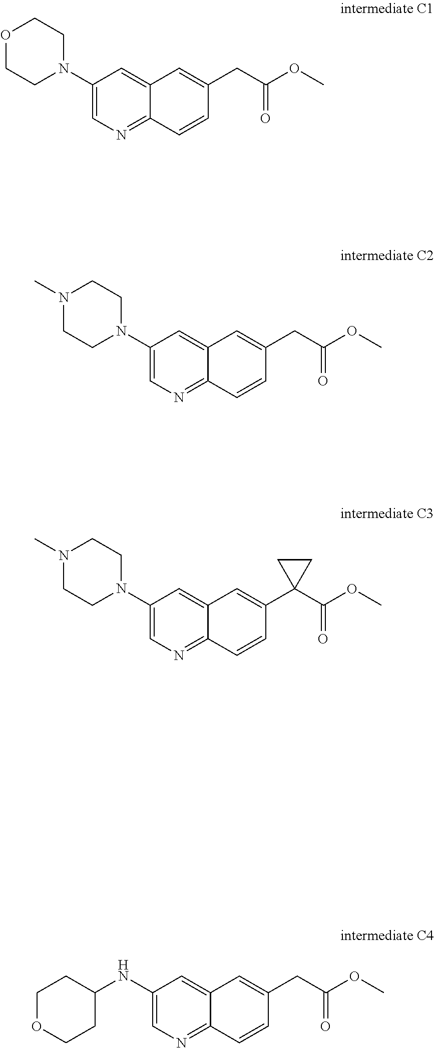

Intermediates C1 to C4 were prepared using the same procedure as described for intermediate C.

### Intermediate D

### Methyl 2-(3-(4-hydroxypiperidin-1-yl)quinolin-6-yl)acetate

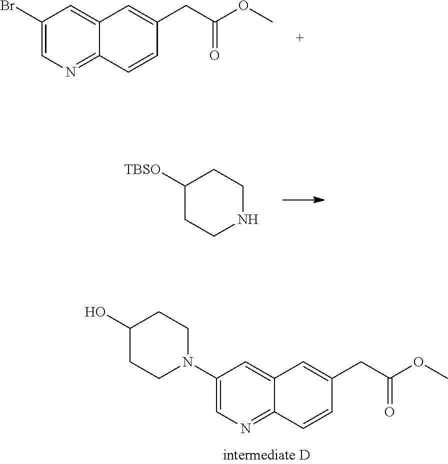 [SMILES: COC(=O)Cc1ccc2ncc(Br)cc2c1 ; CC(C)(C)[Si](C)(C)OC1CCNCC1 ; COC(=O)Cc1ccc2ncc(N3CCC(O)CC3)cc2c1]

A solution of methyl 2-(3-bromoquinolin-6-yl)acetate (2.28 g, 8.14 mmol) and 4-(tert-butyldimethylsilyloxy)piperidine (2.63 g, 12.21 mmol) in toluene (50 mL) was purged with argon for 3 min, followed by addition of Pd₂(dba)₃ (745 mg, 0.814 mmol), Xantphos (942 mg, 1.628 mmol) and t-BuONa (1.564 g, 16.28 mmol) sequentially. The mixture was purged with argon for another half min. The reaction mixture was stirred at 115° C. for 2 h under argon. Then the reaction mixture was cooled to rt and the solvent was removed under reduced pressure. The residue was diluted with methanol (20 mL), then SOCl₂ (3 mL) was added dropwise and the reaction mixture was stirred at rt overnight. The solvent methanol was removed and the residue was dissolved in water, neutralized with NaHCO₃ aqueous solution, extracted with dichloromethane. The organic layers were combined, dried over Na₂SO₄ and concentrated under reduced pressure. The residue was purified by column chromatography on silica gel (ethyl acetate) to afford 290 mg (12%) of the title compound. LCMS (method A): [MH]⁺=301, t_{R}=1.676 min.

### Intermediate E

### Methyl 2-(5,7-difluoro-3-(1-methyl-1H-pyrazol-4-yl)quinolin-6-yl)propanoate

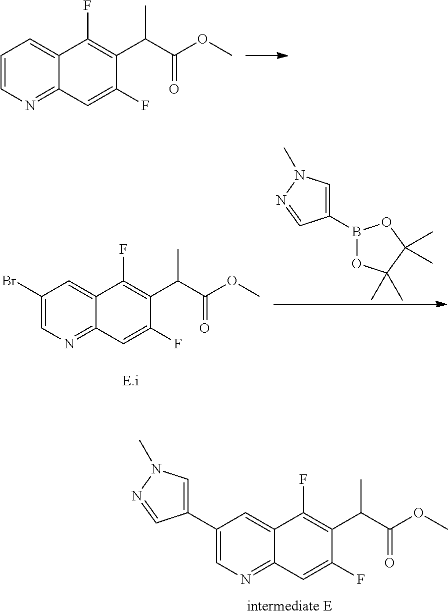

### Methyl 2-(3-bromo-5,7-difluoroquinolin-6-yl)propanoate (E.i)

A solution of 2-(5,7-difluoro-quinolin-6-yl)-propionic acid methyl ester (380 mg, 1.51 mmol) in carbon tetrachloride (6 mL) was added bromine (0.17 mL, 3.30 mmol) at rt. The reddish reaction mixture was heated to reflux, and then cooled to rt. Pyridine (0.3 mL, 3.71 mmol) was added, and the reaction mixture was heated under reflux for 1 h. LCMS showed most starting material was consumed. The mixture was cooled to rt, diluted with CH₂Cl₂. Saturated aqueous NaHCO₃ solution was added carefully, and the mixture was extracted with CH₂Cl₂, dried, concentrated, and purified by column chromatography to afford 291 mg (58% yield) of the title compound as white solid. LCMS (method A): [MN]⁺=331, t_{R}=2.61 min.

### Methyl 2-(5,7-difluoro-3-(1-methyl-1H-pyrazol-4-yl)quinolin-6-yl)propanoate (Inter-mediate E)

To a mixture of 1-methyl-4-(4,4,5,5-tetramethyl-1,3,2-dioxaborolan-2-yl)-1H-pyrazole (841 mg, 4.04 mmol), methyl 2-(3-bromo-5,7-difluoroquinolin-6-yl)propanoate (890 mg, 2.70 mmol), and sodium carbonate (571 mg, 5.39 mmol) in dioxane (40 mL) was added water (3.5 mL), and the mixture was bubbled with argon for 10 min. PdCl₂(dppf) CH₂Cl₂ (220 mg, 0.270 mmol) was added, and the reaction mixture was heated at 100° C. for 4 h. LCMS showed the reaction was complete. The reaction mixture was diluted with EtOAc, washed successively with saturated NaHCO₃ aqueous solution, water, brine, dried, concentrated, and purified by column chromatography to afford 218 mg of the title compound as pale yellow solid. ¹H-NMR (400 MHz, CDCl₃) δ ppm 9.10 (s, 1H), 8.42 (s, 1H), 7.93 (s, 1H), 7.84 (s, 1H), 7.66-7.71 (m, 1H), 4.29 (q, 1H), 4.03 (s, 3H), 3.74 (s, 3H), 1.63 (d, 3H). LCMS (method A): [MH]⁺=332, t_{R}=2.28 min.

### Intermediate E1

### Methyl 2-(5,7-difluoro-3-(1-methyl-1H-pyrazol-4-yl)quinolin-6-yl)acetate

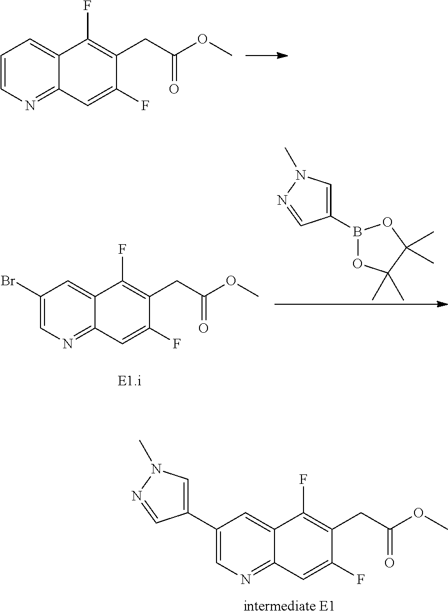

Intermediate E1 was prepared using the same procedure as described for intermediate E starting from methyl 2-(5,7-difluoroquinolin-6-yl)acetate (380 mg, 1.602 mmol) to afford 291 mg of methyl 2-(3-bromo-5,7-difluoroquinolin-6-yl)acetate (E1.i) as white solid. LCMS (method A): [MH]⁺=331, t_{R}=2.61 min. A mixture of 1-methyl-4-(4,4,5,5-tetramethyl-1,3,2-dioxaborolan-2-yl)-1H-pyrazole (247 mg, 1.186 mmol), E1.i (250 mg, 0.791 mmol), and sodium carbonate (168 mg, 1.582 mmol) was reacted to afford 218 mg of the title compound E1 as pale yellow solid. ¹H-NMR (400 MHz, CDCl₃) δ ppm 9.10 (s, 1H), 8.40 (s, 1H), 7.93 (s, 1H), 7.83 (s, 1H), 7.67 (d, 1H), 4.03 (s, 3H), 3.94 (s, 2H), 3.77 (s, 3H). LCMS (method A): [MH]⁺=318, t_{R}=2.31 min.

### Intermediate F

### Methyl 2-(3-(3,5-dimethylisoxazol-4-yl)-5,7-difluoroquinolin-6-yl)acetate

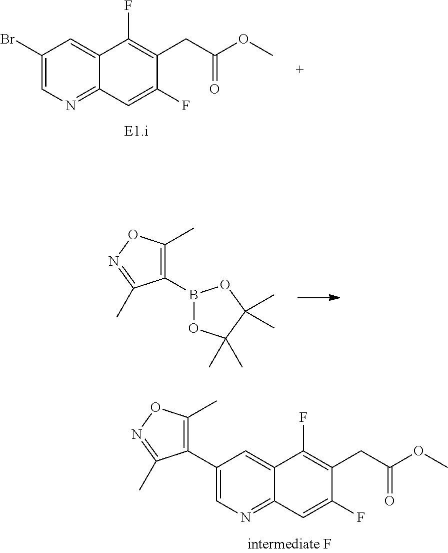

A mixture of 1-methyl-4-(4,4,5,5-tetramethyl-1,3,2-dioxaborolan-2-yl)-1H-pyrazole (318 mg, 1.424 mmol), Methyl 2-(3-bromo-5,7-difluoroquinolin-6-yl)acetate (E1.i) (300 mg, 0.949 mmol), and sodium carbonate (201 mg, 1.898 mmol) in dioxane (15 mL) was added water (1.5 mL), and the mixture was bubbled with argon for 3 min. PdCl₂(dppf)CH₂Cl₂ (78 mg, 0.095 mmol) was added. The reaction vial was sealed and heated at 100° C. for 5 h. LCMS showed the reaction was complete. The reaction mixture was diluted with EtOAc, washed successively with satd. NaHCO₃ aqueous solution, water, brine, dried, concentrated, and purified by column chromatography to afford 244 mg of the title compound as gray solid. ¹H-NMR (400 MHz, CDCl₃) δ ppm 8.87 (s, 1H), 8.25 (s, 1H), 7.68 (s, 1H), 3.95 (s, 2H), 3.78 (s, 3H), 2.50 (s, 3H), 2.35 (s, 3H). LCMS (method A): [MH]⁺=333, t_{R}=2.44 min.

### Intermediate G

### Methyl 2-(3-cyclohexyl-5,7-difluoroquinolin-6-yl)acetate

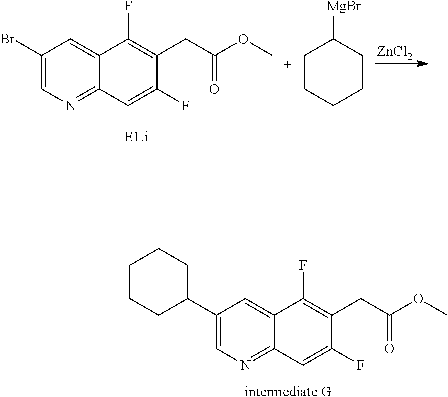

Anhydrous ZnCl₂ (1.68 g, 12.34 mmol) was dissolved in dry degassed NMP (2.0 mL) in a 3-neck flask while heated at 100° C. (oil bath) under N₂ and the resulting solution was allowed to cooled to rt. One neck of the flask was connected with a distillation set-up. To the above solution was added cyclohexylmagnesium (2.0 M in Et₂O) via syringe. The reaction was exothermic and the Et₂O was evaporated. After the completion of addition, the viscous mixture was stirred at rt for 5 min before elevating the temperature to 100° C. to allow for the complete evaporation of Et₂O to give an unstirrable solid. After cooled to rt, methyl 2-(3-bromo-5,7-difluoroquinolin-6-yl)acetate (EU) (1.3 g, 4.11 mmol) and tetrakis palladium (475 mg, 0.411 mmol), NMP (3 mL) were added and the mixture was heated at 100° C. for 1 h. Then the reaction mixture was cooled to rt and poured into ethyl acetate (20 mL) and NaHCO₃ aqueous solution (10 mL), extracted with ethyl acetate. The organic layers were combined, dried over Na₂SO₄ and concentrated under reduced pressure. The residue was purified by column chromatography (10% EtOAc in hexane) to afford 385 mg (29%) of the title compound. ¹H-NMR (400 MHz, CDCl₃) δ ppm 8.87 (s, 1H), 8.27 (s, 1H), 7.78 (d, 1H), 3.93 (s, 2H), 3.76 (s, 3H), 2.79 (t, 1H), 2.05-1.94 (m, 4H), 1.60-1.44 (m, 4H), 1.38-1.27 (m, 2H). LCMS (method A): [MH]⁺=320, t_{R}=2.769 min.

### Intermediate I

### Methyl 3-mercapto-[1,2,4]triazolo[4,3-b]pyridazine-6-carboxylate

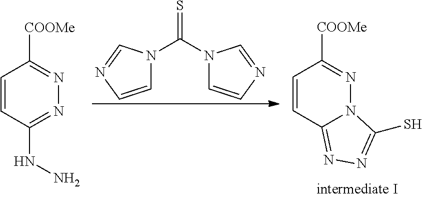

To a solution of methyl 4-hydrazinylbenzoate (3 g, 17.84 mmol) in DMF (20 ml) was added 1,1′-Thiocarbonyldiimidazole (3.18 g, 17.84 mmol). The reaction mixture was stirred for 7 hr at 70° C., concentrated under high pressure pump until half of the DMF solvent was evaporated, and then diluted with CH₂Cl₂ (20 ml). The resulting mixture was purified via biotage on silica gel flash chromatography column gradient with 0-50% MeOH/CH₂Cl₂ to give Methyl 3-mercapto-[1,2,4]triazolo[4,3-b]pyridazine-6-carboxylate (3.41 g, 16.24 mmol, 91% yield) as a yellow powder. LCMS (method B): [MN]⁺=211, t_{R}=1.42 min.

### Intermediate J

### 6-(benzyloxy)-3-bromoquinoline

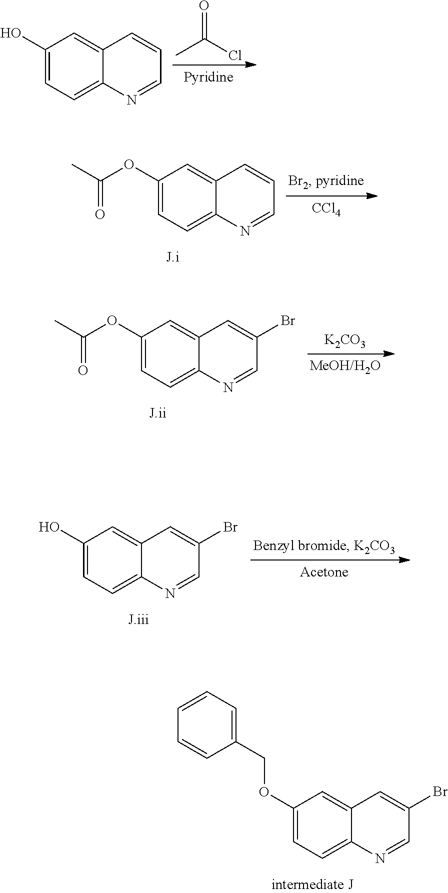

### Quinolin-6-yl acetate (J.i)

To a solution of quinolin-6-ol (4.5 g, 31.0 mmol) and pyridine (3.01 ml, 37.2 mmol) in DCM (50 ml) was added acetyl chloride (2.65 ml, 37.2 mmol) at 0° C. The mixture was then stirred at rt for 8 h. The reaction was quenched with saturated NaHCO₃ and the mixture was extracted with DCM (30 ml) three times. The combined organic phase was washed with brine and dried over anhydrous MgSO₄, filtered and concentrated to give the title compound J.i (5.0 g, 68.9% yield), which was used directly in next step. LCMS (method B): [MH]⁺=188, t_{R}=1.64 min.

### 3-Bromoquinolin-6-yl acetate (J.ii)

To a solution of J.i (5 g, 26.7 mmol) and pyridine (6.48 ml, 80 mmol) in CCl₄ (100 ml) was added Br₂ (4.13 ml, 80 mmol) at 0° C. The resultant brown suspension was then heated at 90° C. for 3 h. After being cooled to rt, the mixture was diluted with DCM and water. The organic phase was separated and washed with water and brine, dried over anhydrous MgSO₄, filtered and concentrated. The residue was purified by silica gel chromatography with Hex/EA (from 100% to 90%) to afford the title compound J.ii as white solid (3.2 g, 40.5% yield). ¹H-NMR (400 MHz, DMSO-d₆) δ ppm 8.95 (s, 1H), 8.73 (s, 1H), 8.08 (d, 1H), 7.74 (d, 1H), 7.62 (dd, 1H), 2.34 (s, 3H). LCMS (method B): [M H]⁺=267, t_{R}=2.29 min.

### 3-Bromoquinolin-6-ol (J.iii)

A solution of J.ii (1 g, 3.76 mmol) and K₂CO₃ (1.04 g, 7.52 mmol) in MeOH/H₂O (5 mL/3 mL) was stirred at rt for 2 hours. The reaction mixture was concentrated under reduced pressure to afford a crude solid which was further purified by washing with water, dried under vacuum to give the title compound J.iii as white solid (760 mg, yield 86%). LCMS (method B): [M+H]⁺=224, t_{R}=2.29 min.

### 6-(Benzyloxy)-3-bromoquinoline (intermediate J)

A solution of J.iii (760 mg, 3.39 mmol), benzyl bromide (0.44 mL, 3.73 mmol) and K₂CO₃ (563 mg, 4.07 mmol) in acetone (20 mL) was stirred at rt overnight. The reaction mixture was concentrated under reduced pressure. The crude product was purified by chromatography (eluting with 20% EtOAc in haxane) to give the title compound as white solid (970 mg, yield 89%). ¹H-NMR (400 MHz, DMSO-d₆) δ ppm 8.76 (d, 1H), 8.23 (d, 1H), 8.05 (d, 1H), 7.49˜7.34 (m, 6H), 7.08 (d, 1H), 5.20 (s, 2H). LCMS (method B): [M+H]⁺=314, t_{R}=2.91 min.

### Intermediate Q1

### (S)-3-(3-(dimethylamino)pyrrolidin-1-yl)quinolin-6-yl trifluoromethanesulfonate

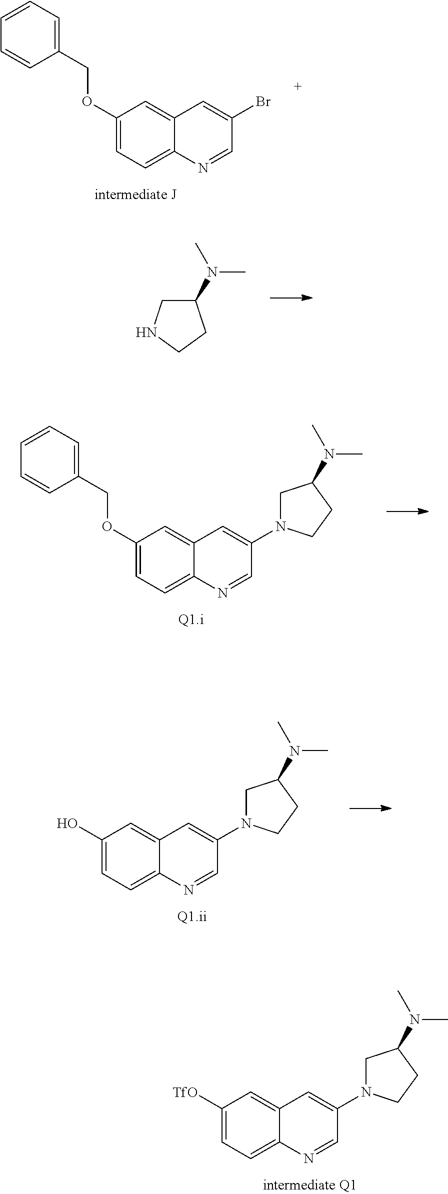

### (S)-1-(6-(benzyloxy)quinolin-3-yl)-N,N-dimethylpyrrolidin-3-amine (Q1.i)

A mixture of Intermediate J (450 mg, 1.43 mmol), (S)—N,N-dimethylpyrrolidin-3-amine (196 mg, 1.72 mmol), Pd₂(dba)₃ (65.6 mg, 0.072 mmol), Xantphos (83 mg, 0.143 mmol) and KO^{t}Bu (241 mg, 2.15 mmol) in toluene (4.5 mL) was bubbled with argon for 20 min. The result mixture was heated at 110° C. overnight. The solution was cooled to rt and the solvent was removed under reduced pressure. The residue was diluted with water, extracted with DCM three times. The combined organic phase was dried over anhydrous Na₂SO₄, filtered and concentrated. The crude product was purified by chromatography (eluting with 5% MeOH in DCM) to give the title compound as yellow solid (435 mg, yield 83%). LCMS (method B): [M+H]⁺=348, t_{R}=1.72 min.

### (S)-3-(3-(dimethylamino)pyrrolidin-1-yl)quinolin-6-ol (Q1.ii)

To a solution of Q1.i (435 mg, 1.43 mmol) in MeOH (10 mL) was added 10% Pd/C (133 mg, 0.125 mmol). The mixture was reacted under hydrogen atmosphere overnight. The result mixture was filtrated. The filtrate was concentrated under reduced pressure, dried in vacuum to give the title compound as yellow solid (280 mg, yield 78%). ¹H-NMR (400 MHz, DMSO-d₆) δ ppm 9.68 (s, 1H), 8.29 (d, 1H), 7.63 (d, 1H), 6.90˜6.87 (m, 3H), 3.61˜3.57 (m, 1H), 3.53˜3.49 (m, 1H), 3.38˜3.32 (m, 1H), 3.16˜3.12 (m, 1H), 2.83˜2.79 (m, 1H), 2.22˜2.16 (m, 7H), 1.85˜1.80 (m, 1H).

### (S)-3-(3-(Dimethylamino)pyrrolidin-1-yl)quinolin-6-yl trifluoromethanesulfonate (intermediate Q1)

To a suspension of Q1.ii (280 mg, 0.979 mmol) and pyridine (0.2 mL, 2.45 mmol) in DCM (5 mL) was added Tf₂O (0.15 mL, 1.96 mmol) dropwise under ice-bath. The reaction was stirred at rt overnight, then quenched by saturated NaHCO₃ and concentrated under reduced pressure. The residue was diluted with water, extracted with DCM three times. The combined organic phase was washed with brine, dried over anhydrous Na₂SO₄, filtered and concentrated. The crude product was purified by chromatography (eluting with 5% MeOH in DCM) to give the title compound as yellow solid (130 mg, yield 46%). ¹H-NMR (400 MHz, DMSO-d₆) δ ppm 8.65 (d, 1H), 7.97 (d, 1H), 7.83 (s, 1H), 7.37 (d, 1H), 7.24 (s, 1H), 4.09˜4.06 (m, 2H), 3.66 (t, 1H), 3.59 (t, 1H), 3.44˜3.38 (m, 1H), 3.24˜3.20 (m, 1H), 2.90 (broad, 1H), 2.26 (s, 6H), 1.93˜1.83 (m, 1H). LCMS (method B): [M+H]⁺=390, t_{R}=2.75 min.

### Intermediate Q2

### 3-((Diethylamino)methyl)quinolin-6-yl trifluoromethanesulfonate

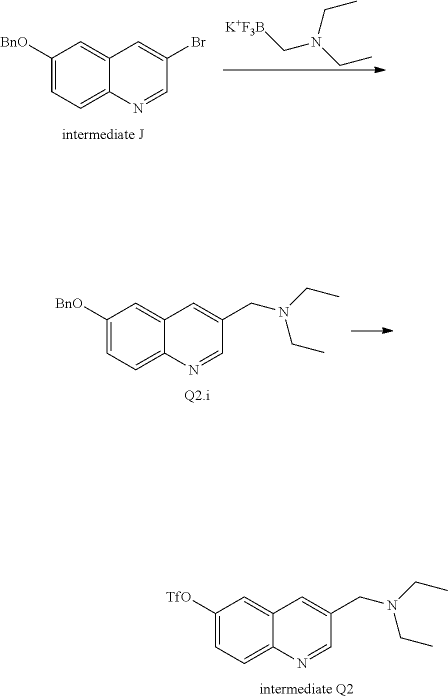

### N-((6-(benzyloxy)quinolin-3-yl)methyl)-N-ethylethanamine (Q2.i)

A mixture of Intermediate J (2.27 g, 7.25 mmol), potassium trifluoro[(N,N-diethylamino)methyl]borate (1.4 g, 7.25 mmol), dibromobis(tri-tert-butylphosphine) dipalladium(I) (332 mg, 0.36 mmol), and cesium carbonate (2.84 g, 8.70 mmol) in dioxane (30 mL)/H₂O (3 mL) was bubbled with argon for 20 min. The resulting mixture was heated at 80° C. and stirred for 3 h. Then the reaction mixture was cooled to r.t, water was added and extracted with DCM three times. The combined organic phase was dried over anhydrous Na₂SO₄, filtered, concentrated and purified by chromatography column (eluting with 5% MeOH in DCM) to give the title compound as yellow solid (1.37 g, yield 59%). LCMS (method A): [M+H]⁺=321, t_{R}=5.21 min.

### 3-((diethylamino)methyl)quinolin-6-yl trifluoromethanesulfonate (intermediate Q2)

To a solution of Q2.i (1.37 g, 4.28 mmol) in MeOH (25 mL) was added 10% Pd/C (450 mg, 0.42 mmol). The mixture was stirred under hydrogen atmosphere overnight, filtrated, concentrated under reduced pressure, and dried in vacuum to give the quinolin amine as yellow solid (530 mg, yield 50%). LCMS (method A): [M+H]⁺=321, t_{R}=0.93 min. To a suspension of the obtained (3-((diethylamino)methyl)quinolin-6-ol (530 mg, 2.38 mmol) and pyridine (0.77 mL, 9.55 mmol) in DCM (25 mL) was added Tf₂O (0.81 mL, 4.78 mmol) dropwise under ice-bath. The reaction mixture was stirred at room temperature overnight, quenched with saturated NaHCO₃ and concentrated under reduced pressure. The residue was diluted with water, extracted with DCM three times. The combined organic phases were washed with brine, dried over anhydrous Na₂SO₄, filtrated, concentrated and purified by chromatography column (eluting with 5% MeOH in DCM) to give the title compound Q2 as yellow solid (510 mg, yield 49%). LCMS (method A): [M+H]⁺=363, t_{R}=1.71 min.

### Intermediate Q3 to Q7

### 3-(Morpholin-4-yl)-quinolin-6-yl trifluoromethanesulfonate (Q3)

### 3-(4-Methyl-piperazin-1-yl)-quinolin-6-yl trifluoromethanesulfonate (Q4)

### 3-(4-Hydroxy-piperidin-1-yl)-quinolin-6-yl trifluoromethanesulfonate (Q5)

### 3-(Tetrahydro-pyran-4-ylamino)-quinolin-6-yl trifluoromethanesulfonate (Q6)

### 3-(Morpholin-4-ylmethyl)-quinolin-6-yl trifluoromethanesulfonate (Q7)

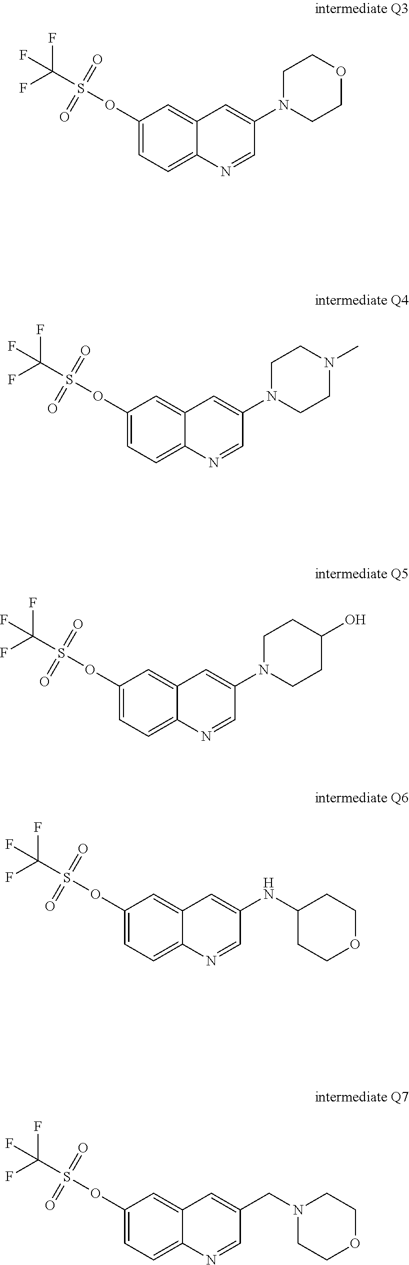 [SMILES: O=S(=O)(Oc1ccc2ncc(N3CCOCC3)cc2c1)C(F)(F)F ; CN1CCN(c2cnc3ccc(OS(=O)(=O)C(F)(F)F)cc3c2)CC1 ; O=S(=O)(Oc1ccc2ncc(N3CCC(O)CC3)cc2c1)C(F)(F)F ; O=S(=O)(Oc1ccc2ncc(NC3CCOCC3)cc2c1)C(F)(F)F ; O=S(=O)(Oc1ccc2ncc(CN3CCOCC3)cc2c1)C(F)(F)F]

Intermediates Q3 to Q7 were prepared from intermediate J using the same procedure as described for Q1 or Q2.

### SYNTHESIS OF EXAMPLES

### Example 1

### Method 1A

### (E)-1-(3-{1-[3-(1-Methyl-1H-pyrazol-4-yl)quinolin-6-yl]-ethyl}-[1,2,4]triazolo[4,3-b]pyridazin-6-yl)-ethanone oxime

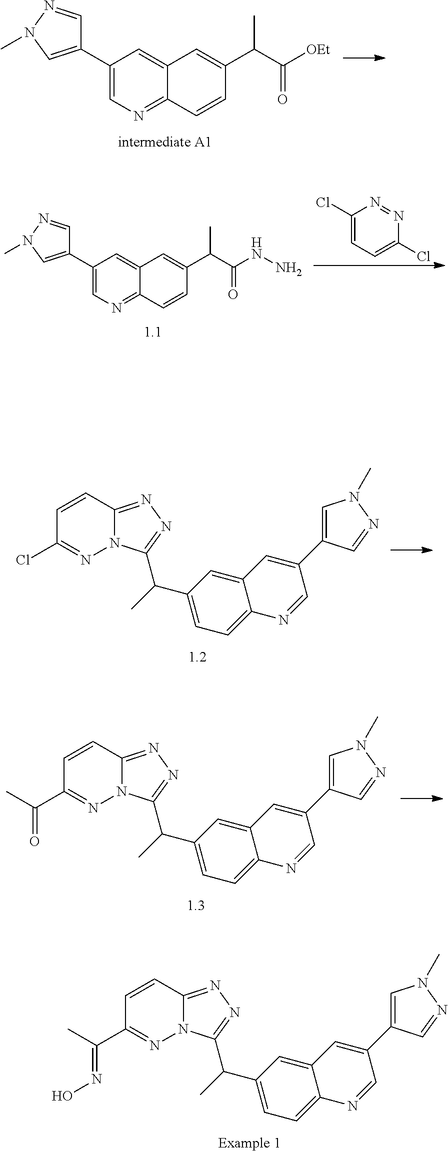 [SMILES: CCOC(=O)C(C)c1ccc2ncc(-c3cnn(C)c3)cc2c1 ; CC(C(=O)NN)c1ccc2ncc(-c3cnn(C)c3)cc2c1 ; Clc1ccc(Cl)nn1 ; CC(c1ccc2ncc(-c3cnn(C)c3)cc2c1)c1nnc2ccc(Cl)nn12 ; CC(=O)c1ccc2nnc(C(C)c3ccc4ncc(-c5cnn(C)c5)cc4c3)n2n1 ; C/C(=N\O)c1ccc2nnc(C(C)c3ccc4ncc(-c5cnn(C)c5)cc4c3)n2n1]

### 2-[3-(1-Methyl-1H-pyrazol-4-yl)quinolin-6-yl]-propionic acid hydrazide (1.1)

To a suspension 2-[3-(1-methyl-1H-pyrazol-4-yl)quinolin-6-yl]-propionic acid ethyl ester (Intermediate A1, 2.8 g, 9.05 mmol) in MeOH (10 mL) was added hydrazine hydrate (2 mL, 64.3 mmol) and then the mixture was heated at reflux for about 5 h. The solution was cooled to rt and the solvent was removed under reduced pressure to afford a white precipitate. It was washed with a little MeOH to give the title compound 1.1 as white solid (2.2 g, yield 83%). LCMS (method A): [M+H]⁺=296, t_{R}=1.49 min.

### 6-[1-(6-Chloro-[1,2,4]triazolo[4,3-b]pyridazin-3-yl)-ethyl]-3-(1-methyl-1H-pyrazol-4-yl)quinoline (1.2)

A suspension of (1.1) (2.4 g, 8.13 mmol) and 3,6-Dichloro-pyridazine (1.816 g, 12.19 mmol) in n-BuOH (25 mL) was sealed in a microwave vial and was heated at 140° C. for about 2 h. The solvent was removed and the residue was purified by silica gel chromatography (eluting with 5% MeOH in DCM) to give the title compound 1.2 as yellow solid (1.8 g, yield 57%). LCMS (method A): [M+H]⁺=390, t_{R}=2.09 min.

### 1-(3-{1-[3-(1-Methyl-1H-pyrazol-4-yl)quinolin-6-yl]-ethyl}-[1,2,4]triazolo[4,3-b]pyridazin-6-yl)-ethanone (1.3)

A mixture of (1.2) (400 mg, 1.026 mmol) and Pd(PPh₃)₂Cl₂ (720 mg, 1.026 mmol) in dioxane (10 mL) was bubbled with argon for about 20 min, then tributyl-(1-ethoxy-vinyl)-stannane was added and was bubbled for further 3 min. The resultant mixture was heated at 90° C. overnight. The solution was cooled to rt and diluted with MeOH and treated with HCl (3N) overnight. The solvent was removed and the residue was purified by chromatography to give the ketone 1.3 as yellow solid (80 mg, yield 18%). LCMS (method A): [M+H]⁺=398, t_{R}=1.95 min.

### (E)-1-(3-{1-[3-(1-Methyl-1H-pyrazol-4-yl)quinolin-6-yl]-ethyl}-[1,2,4]triazolo[4,3-b]pyridazin-6-yl)-ethanone oxime (Example 1)

To a mixture of (1.3) (30 mg, 0.075 mmol) and hydroxylamine (26.2 mg, 0.377 mmol) in MeOH (3 mL) was added a drop of HCl (1N). It was stirred at rt overnight. After concentration, the residue was purified by prep-HPLC to give the title compound as brown solid (15 mg, 48%). ¹H-NMR (400 MHz, DMSO-d6) δ ppm 12.5 (s, 1H), 9.11 (d, 1H), 8.41 (d, 1H), 8.36 (s, 1H), 8.23 (d, 1H), 9.07 (s, 1H), 7.92 (d, 1H), 7.80 (d, 1H), 7.71 (dd, 2H), 5.03 (q, 1H), 3.89 (s, 3H), 2.13 (s, 3H), 1.91 (d, 3H). LCMS (method A): [M+H]⁺=413, t_{R}=2.16 min.

### Example 2

### (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-ethyl-oxime

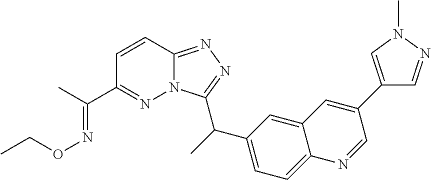 [SMILES: CCO/N=C(\C)c1ccc2nnc(C(C)c3ccc4ncc(-c5cnn(C)c5)cc4c3)n2n1]

The title compound was prepared using the same procedure as described in the synthesis of Example 1 by using the equivalent amount of O-ethylhydroxylamine instead of the hydroxylamine. ¹H-NMR (400 MHz, DMSO-d6) δ ppm 9.11 (s, 1H), 8.41 (s, 1H), 8.35 (s, 1H), 8.25 (d, 1H), 8.06 (s, 1H), 7.91 (d, 1H), 7.81 (s, 1H), 7.70 (d, 2H), 5.04 (m, 1H), 4.23 (q, 2H), 3.89 (s, 3H), 2.15 (s, 3H), 1.92 (s, 3H), 1.26 (t, 3H). LCMS (method A): [M+H]⁺=441, t_{R}=2.49 min.

### Example 3

### (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-methyl-oxime

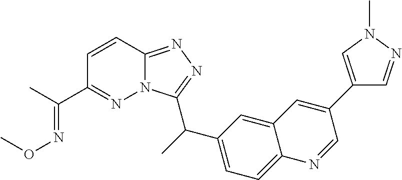 [SMILES: CO/N=C(\C)c1ccc2nnc(C(C)c3ccc4ncc(-c5cnn(C)c5)cc4c3)n2n1]

The title compound was prepared using the same procedure as described in the synthesis of Example 1 by using the equivalent amount of O-methylhydroxylamine instead of the hydroxylamine. ¹H-NMR (400 MHz, DMSO-d6) δ ppm 9.11 (s, 1H), 8.40 (s, 1H), 8.35 (s, 1H), 8.24 (d, 1H), 8.06 (s, 1H), 7.91 (s, 1H), 7.81 (s, 1H), 7.68 (m, 2H), 5.4 (q, 1H), 4.60 (s, 3H), 3.89 (s, 3H), 2.15 (s, 3H), 1.92 (s, 3H). LCMS (method A): [M+H]⁺=427, t_{R}=2.36 min. Chiral separation (method C) provided enantiomeric pure compounds Example 3-(S) and Example 3-(R).

### Example 4

### (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-cyclopropylmethyl-oxime

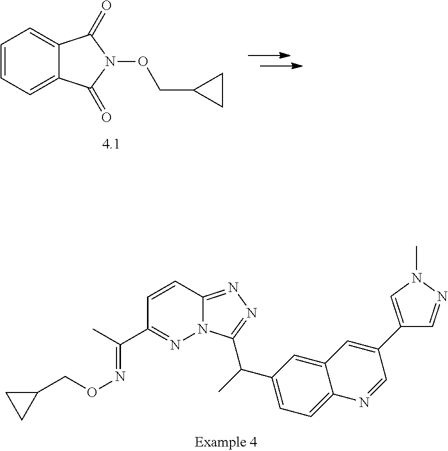 [SMILES: O=C1c2ccccc2C(=O)N1OCC1CC1 ; C/C(=N\OCC1CC1)c1ccc2nnc(C(C)c3ccc4ncc(-c5cnn(C)c5)cc4c3)n2n1]

A mixture of 4.1 (82 mg, 0.377 mmol) and hydrazine hydrate (5.87 μl, 0.189 mmol) in MeOH (3 mL) was heated at reflux for about 3 h. It was filtered and the filtrate was combined with 1-(3-{1-[3-(1-Methyl-1H-pyrazol-4-yl)quinolin-6-yl]-ethyl}-[1,2,4]triazolo[4,3-b]pyridazin-6-yl)-ethanone (Compound 1.3 in Example 1, 30 mg, 0.075 mmol) and a little HCl (1M). The resulting solution was stirred at rt overnight. After concentration, the residue was purified by prep-HPLC to give the title compound as white solid (13 mg, yield 37%). ¹H-NMR (400 MHz, DMSO-d6) δ ppm 9.11 (s, 1H), 8.41 (s, 1H), 8.35 (s, 1H), 8.24 (d, 1H), 8.06 (s, 1H), 7.91 (d, 1H), 7.81 (s, 1H), 7.70 (m, 2H), 5.04 (q, 1H), 4.04 (m, 2H), 3.89 (s, 3H), 2.17 (s, 3H), 1.93 (s, 3H), 1.16 (m, 1H), 0.52 (m, 2H), 0.30 (m, 2H). LCMS (method A): [M+H]⁺=467, t_{R}=2.55 min. Chiral separation (method C) provided enantiomeric pure compounds Example 4-(S) and Example 4-(R).

### Example 5

### (E)-1-(3-{1-[3-(1-Methyl-1H-pyrazol-4-yl)quinolin-6-yl]-ethyl}-[1,2,4]triazolo[4,3-b]pyridazin-6-yl)-ethylidene]-hydrazine

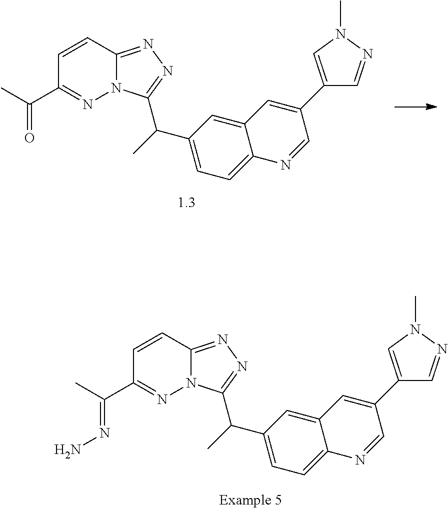 [SMILES: CC(=O)c1ccc2nnc(C(C)c3ccc4ncc(-c5cnn(C)c5)cc4c3)n2n1 ; C/C(=N\N)c1ccc2nnc(C(C)c3ccc4ncc(-c5cnn(C)c5)cc4c3)n2n1]

A mixture of 1-(3-{1-[3-(1-Methyl-1H-pyrazol-4-yl)quinolin-6-yl]-ethyl}-[1,2,4]triazolo[4,3-b]pyridazin-6-yl)-ethanone (Compound 1.3 in Example 1, 164 mg, 0.755 mmol) and hydrazine hydrate (0.012 mL, 0.377 mmol) in MeOH (3 mL) was stirred at rt overnight. After concentration, the residue was purified by prep-HPLC to give the title compound as white solid (6 mg, yield 19%). ¹H-NMR (400 MHz, DMSO-d6) δ ppm 9.11 (s, 1H), 8.42 (s, 1H), 8.36 (s, 1H), 8.06 (s, 2H), 7.91 (d, 1H), 7.81 (s, 1H), 7.74 (d, 1H), 7.69 (d, 1H), 7.48 (s, 2H), 5.00 (q, 1H), 3.90 (s, 3H), 1.99 (s, 3H), 1.91 (s, 3H). LCMS (method A): [M+H]⁺=412, t_{R}=1.89 min.

### Example 6

### Method 1A

### (E)-1-{3-[3-(1-Methyl-1H-pyrazol-4-yl)quinolin-6-ylmethyl]-[1,2,4]triazolo[4,3-b]pyridazin-6-yl}-ethanone O-methyl-oxime

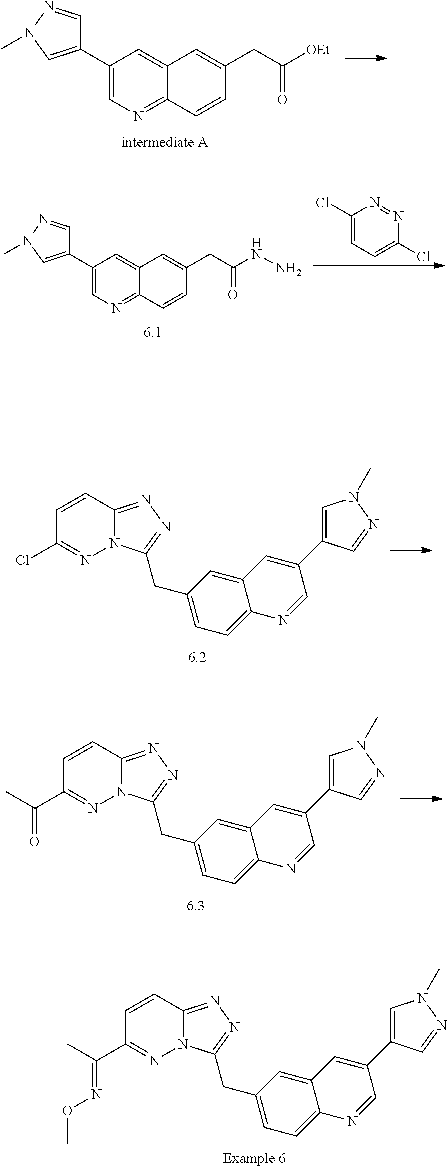 [SMILES: CCOC(=O)Cc1ccc2ncc(-c3cnn(C)c3)cc2c1 ; Cn1cc(-c2cnc3ccc(CC(=O)NN)cc3c2)cn1 ; Clc1ccc(Cl)nn1 ; Cn1cc(-c2cnc3ccc(Cc4nnc5ccc(Cl)nn45)cc3c2)cn1 ; CC(=O)c1ccc2nnc(Cc3ccc4ncc(-c5cnn(C)c5)cc4c3)n2n1 ; CO/N=C(\C)c1ccc2nnc(Cc3ccc4ncc(-c5cnn(C)c5)cc4c3)n2n1]

### [3-(1-Methyl-1H-pyrazol-4-yl)quinolin-6-yl]-acetic acid hydrazide (6.1)

To a solution of 3-(1-methyl-1H-pyrazol-4-yl)quinolin-6-yl]-acetic acid ethyl ester (Intermediate A, 6.8 g, 24.17 mmol) in MeOH (30 mL) was added hydrazine hydrate (2.68 mL, 72.5 mmol). It was heated at reflux overnight. The solution was cooled to rt. The title compound 6.1 was collected as a white solid (6 g, 88%). LCMS (method A): [M+H]⁺=282, t_{R}=2.20 min.

### 6-(6-Chloro-[1,2,4]triazolo[4,3-b]pyridazin-3-ylmethyl)-3-(1-methyl-1H-pyrazol-4-yl)quinoline (6.2)

A suspension of 6.1 (1.6 g, 5.71 mmol) and 3,6-dichloro-pyridazine (1.28 g, 8.56 mmol) in n-BuOH (25 mL) was sealed in a microwave vial and was heated at 140° C. for about 2 h. The solvent was removed and the residue was purified by silica gel chromatography (eluting with 5% MeOH in DCM) to give the title compound as yellow solid (1.8 g, yield 57%). LCMS (method A): [M+H]⁺=376.1, t_{R}=2.06 min.

### 1-{3-[3-(1-Methyl-1H-pyrazol-4-yl)quinolin-6-ylmethyl]-[1,2,4]triazolo[4,3-b]pyridazin-6-yl}-ethanone (6.3)

A mixture of 6.2 (1.6 g, 4.26 mmol) and Pd(PPh₃)₂Cl₂ (448 mg, 0.64 mmol) in dioxane (20 mL) was bubbled with argon for about 20 min, then tributyl-(1-ethoxy-vinyl)-stannane (2.54 mL, 8.52 mmol) was added and it was bubbled with argon for further 3 min. The resulting mixture was heated at 90° C. overnight. The solution was cooled to rt and diluted with MeOH and treated with HCl (3N) overnight. The solvent was removed and the residue was purified by chromatography to give ketone 6.3 as yellow solid (1.0 g, yield 60%). LCMS (method A): [M+H]⁺=384, t_{R}=1.91 min.

### 1-{3-[3-(1-Methyl-1H-pyrazol-4-yl)quinolin-6-ylmethyl]-[1,2,4]triazolo[4,3-b]pyridazin-6-yl}-ethanone O-methyl-oxime (Example 6)

To a solution of 6.3 (60 mg, 0.078 mmol) in MeOH (3 mL) was added O-methylhydroxylamine hydrochloride (6.53 mg, 0.078 mmol) and a drop of 1N HCl. The solution was stirred at rt overnight. After concentration, the residue was purified by prep-HPLC to give the title compound as white solid (12 mg, yield 37%). ¹H-NMR (400 MHz, DMSO-d6) δ ppm 9.13 (s, 1H), 8.37 (d, 2H), 8.28 (s, 1H), 8.07 (s, 1H), 7.92 (d, 1H), 7.74 (s, 1H), 7.71 (m, 2H), 4.74 (s, 2H), 4.04 (s, 3H), 3.89 (s, 3H), 2.24 (s, 3H). LCMS (method A): [M+H]⁺=413, t_{R}=2.29 min.

### Example 7

### (E)-1-{3-[3-(1-Methyl-1H-pyrazol-4-yl)quinolin-6-ylmethyl]-[1,2,4]triazolo[4,3-b]pyridazin-6-yl}-ethanone O-cyclopropylmethyl-oxime

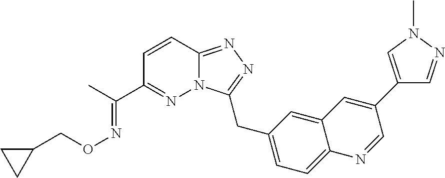 [SMILES: C/C(=N\OCC1CC1)c1ccc2nnc(Cc3ccc4ncc(-c5cnn(C)c5)cc4c3)n2n1]

The title compound was prepared using the same procedure as described in the synthesis of Example 6 from 6.3 by using the equivalent amount of O-cyclopropylmethyl-hydroxylamine instead of the O-methylhydroxylamine. ¹H-NMR (400 MHz, DMSO-d6) δ ppm 9.13 (s, 1H), 8.38 (d, 2H), 8.27 (d, 1H), 8.07 (s, 1H), 7.92 (d, 1H), 7.83 (s, 1H), 7.72 (m, 2H), 4.74 (s, 2H), 4.08 (d, 2H), 3.90 (s, 3H), 2.26 (s, 3H), 1.21 (m, 1H), 0.54 (m, 2H), 0.33 (m, 2H). LCMS (method A): [M+H]⁺=453, t_{R}=2.50 min.

### Example 8

### (E)-1-{3-[3-(1-Methyl-1H-pyrazol-4-yl)quinolin-6-ylmethyl]-[1,2,4]triazolo[4,3-b]pyridazin-6-yl}-ethanone O-ethyl-oxime

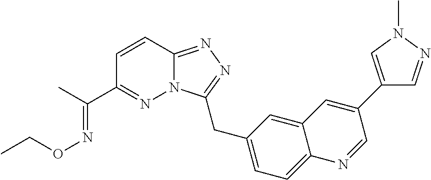 [SMILES: CCO/N=C(\C)c1ccc2nnc(Cc3ccc4ncc(-c5cnn(C)c5)cc4c3)n2n1]

The title compound was prepared using the same procedure as described in the synthesis of Example 6 from 6.3 by using the equivalent amount of O-ethylhydroxylamine instead of the O-methylhydroxylamine. ¹H-NMR (400 MHz, DMSO-d6) δ ppm 9.13 (s, 1H), 8.38 (d, 1H), 8.27 (1, 2H), 8.07 (s, 1H), 7.92 (d, 1H), 7.83 (s, 1H), 7.76 (m, 2H), 4.74 (m, 2H), 4.29 (q, 2H), 3.90 (s, 3H), 2.19 (s, 3H), 2.19 (s, 3H), 1.23 (t, 3H). LCMS (method A): [M+H]⁺=427, t_{R}=2.40 min.

### Example 9

### Method 2

### (E)-1-{3-[1-(3-Morpholin-4-ylmethyl-quinolin-6-yl)-ethyl]-[1,2,4]triazolo[4,3-b]pyridazin-6-yl}-ethanone O-cyclopropylmethyl-oxime

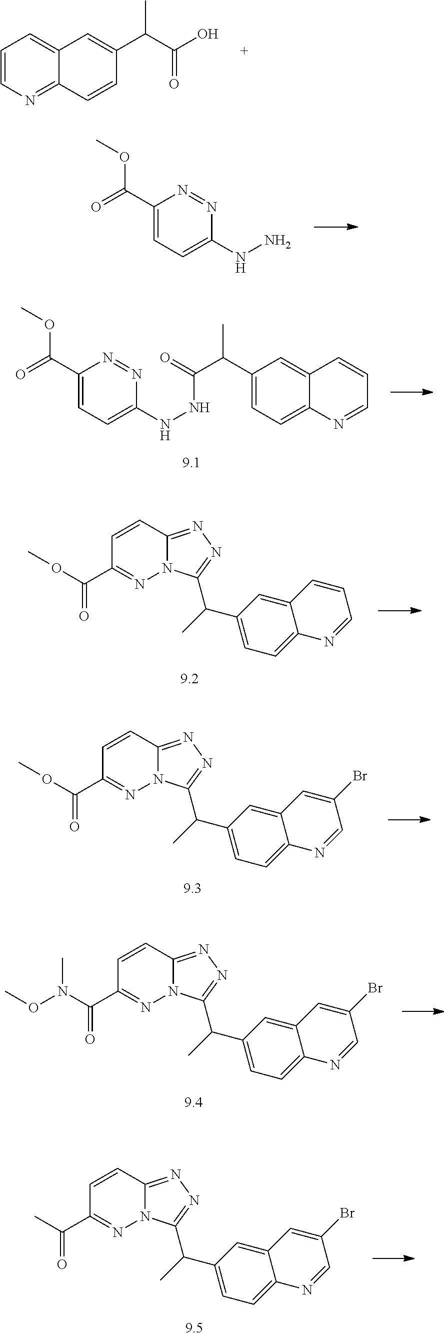

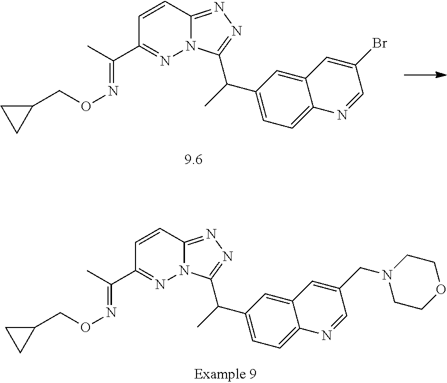 [SMILES: C/C(=N\OCC1CC1)c1ccc2nnc(C(C)c3ccc4ncc(Br)cc4c3)n2n1 ; C/C(=N\OCC1CC1)c1ccc2nnc(C(C)c3ccc4ncc(CN5CCOCC5)cc4c3)n2n1]

### 6-[N′-(2-Quinolin-6-yl-propionyl)-hydrazino]-pyridazine-3-carboxylic acid methyl ester (9.1)

To a solution of 2-quinolin-6-yl-propionic acid (3.2 g, 15.9 mmol) in DCM (20 mL) was added DIPEA (5.55 mL, 31.8 mmol), HATU (6.65 g, 17.49 mmol) and 6-Hydrazino-pyridazine-3-carboxylic acid methyl ester (2.67 g, 15.9 mmol). Then the mixture was stirred at rt for about 1 hr. The mixture was diluted with DCM and washed with NaOH (1 N). The organic phase was dried over anhydrous MgSO₄. Then filtered and concentrated and purified by silica gel chromatography (eluted with 5% MeOH in DCM) to give the title compound as yellow solid (4.2 g, yield 75%). LCMS (method A): [M+H]⁺=408, t_{R}=2.00 min.

### 3-(1-Quinolin-6-yl-ethyl)-[1,2,4]triazolo[4,3-b]pyridazine-6-carboxylic acid methyl ester (9.2)

A suspension of 9.1 (4.2 g, 11.95 mmol) in HOAc (25 mL) was sealed and heated at 100° C. for 3 hr. The solvent was removed under reduced pressure. The residue was diluted with EA, and washed with saturated NaHCO₃ aqueous solution. The water phase was extracted with EA for 2 times. The combined organic phase was dried over anhydrous MgSO₄, then filtered and concentrated. The residue was purified by silica gel chromatography (eluted with 3% MeOH in DCM) to give the title compound as yellow solid (3.1 g, yield 78%). LCMS (method A): [M+H]⁺=334, t_{R}=1.75 min.

### 3-[1-(3-Bromo-quinolin-6-yl)-ethyl]-[1,2,4]triazolo[4,3-b]pyridazine-6-carboxylic acid methyl ester (9.3)

To a suspension of 9.2 (2.5 g, 7.50 mmol) in CCl₄ (200 mL) was added pyridine (1.21 mL, 15.0 mmol) and bromine (0.58 mL, 11.25 mmol) successively. Then the suspension was heated at reflux for 2 hr. Before the suspension was cooled down, it was filtered via silica and the filtrate was concentrated. The residue was purified by chromatography to give the title compound as brown solid (1.1 g, 35%). LCMS (method A): [M+H]⁺=412/414, t_{R}=2.36 min.

### 3-[1-(3-Bromo-quinolin-6-yl)-ethyl]-[1,2,4]triazolo[4,3-b]pyridazine-6-carboxylicacid methoxy-methyl-amide (9.4)

A solution of 9.3 (1.1 g, 2.67 mmol) in MeOH/H₂O (15 mL, v/v=5:1) was added LiOH (0.192 g, 3 mmol). The mixture was stirred at rt overnight. Then N-methylmorpholine (0.293 mL, 2.67 mmol) and HATU (1.02 g, 2.67 mmol) and N,O,-dimethylamine hydrochlodide (260 mg, 2.67 mmol) was added. The mixture was stirred at rt for 5 hr. The reaction was quenched with water. The water phase was extracted with EA and the combined extract was dried over anhydrous MgSO₄. Filtered and the residue was purified by chromatography to give the title compound as yellow solid. (750 mg, yield, 64%). LCMS (method A): [M+H]⁺=441/443, t_{R}=2.22 min.

### 1-{3-[1-(3-Bromo-quinolin-6-yl)-ethyl]-[1,2,4]triazolo[4,3-b]pyridazin-6-yl}-ethanone (9.5)

To a solution of 9.4 (150 mg, 0.340 mmol) in THF (5 mL) was added methylmagnesium iodide (1.360 mL, 4.08 mmol) at −78° C. After addition, the mixture was warmed naturally to rt and was stirred at this temperature for about 2.5 hr. The reaction was quenched with saturated NH₄Cl aqueous solution. THF was removed under reduced pressure. The residue was extracted with EA for 3 times. The organic phase was dried over anhydrous MgSO₄. Filtered and concentrated. The obtained solid (100 mg, yield 74%) was used in next steps without further purification. LCMS (method A): [M+H]⁺=396/398, t_{R}=2.37 min.

### 1-{3-[1-(3-Bromo-quinolin-6-yl)-ethyl]-[1,2,4]triazolo[4,3-b]pyridazin-6-yl}-ethanone O-cyclopropylmethyl-oxime (9.6)

A mixture of 9.5 (160 mg, 0.404 mmol) and O-(cyclopropylmethyl)hydroxylamine (11.36 mg, 0.130 mmol) in MeOH (3 mL) was added a drop of 1N HCl and then the resultant solution was stirred at rt overnight. MeOH was removed and diluted with water. The pH was adjusted to weak base with saturated NaHCO₃ aqueous solution. The water phase was extracted with DCM: IPA(v/v=3:1) for 3 times. The combined extract was dried over anhydrous MgSO₄, then filtered and concentrated. The residue was purified by silica gel chromatography to give the title compound as brown solid (100 mg, 53%). LCMS (method A): [M+H]⁺=465/467, t_{R}=2.81 min.

### 1-{3-[1-(3-Morpholin-4-ylmethyl-quinolin-6-yl)-ethyl]-[1,2,4]triazolo[4,3-b]pyridazin-6-yl}-ethanone O-cyclopropylmethyl-oxime (Example 9)

A mixture of 9.6 (60 mg, 0.103 mmol), potassium (morpholin-4-yl)methyltrifluoroborate (21.36 mg, 0.103 mmol), Pd₂(dba)₃ (18.89 mg, 0.021 mmol), XPhos (19.64 mg, 0.041 mmol) and Cs₂CO₃ (67.2 mg, 0.206 mmol) in THF/H₂O(10:1) (4 mL) was bubbled with argon for about 10 min. The mixture was then heated at 80° C. for 20 h. The solvent was removed under reduced pressure. The residue was purified by prep-HPLC to afford the title compound as white solid (6 mg, 11%). ¹H-NMR (400 MHz, DMSO-d6) 8 ppm. 8.78 (s, 1H), 8.24 (d, 1H), 8.16 (s, 1H), 7.92 (m, 2H), 7.74 (d, 1H), 7.68 (d, 1H), 5.03 (q, 1H), 4.05 (d, 2H), 3.64 (s, 2H), 3.56 (m, 4H), 2.37 (m, 4H), 2.03 (s, 3H), 1.93 (d, 3H), 1.18 (m, 1H), 0.51 (m, 2H), 0.31 (m, 2H). LCMS (method A): [M+H]⁺=486, t_{R}=1.92 min.

### Example 10

### Method 3

### (E)-1-[3-(3-Morpholin-4-yl-quinolin-6-ylsulfanyl)-[1,2,4]triazolo[4,3-b]pyridazin-6-yl]-ethanone oxime

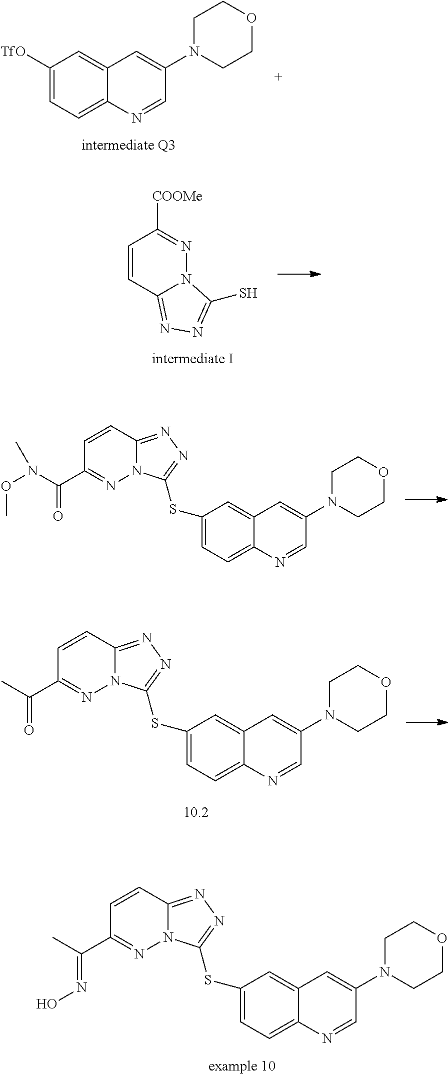 [SMILES: O=S(=O)(Oc1ccc2ncc(N3CCOCC3)cc2c1)C(F)(F)F ; COC(=O)c1ccc2nnc(S)n2n1 ; CON(C)C(=O)c1ccc2nnc(Sc3ccc4ncc(N5CCOCC5)cc4c3)n2n1 ; CC(=O)c1ccc2nnc(Sc3ccc4ncc(N5CCOCC5)cc4c3)n2n1 ; C/C(=N\O)c1ccc2nnc(Sc3ccc4ncc(N5CCOCC5)cc4c3)n2n1]

### N-methoxy-N-methyl-3-((3-morpholin-4-yl-quinolin-6-yl)sulfanyl)-[1,2,4]triazolo[4,3-b]pyridazine-6-carboxamide (10.1)

A mixture of 3-(morpholin-4-yl)-quinolin-6-yl trifluoromethanesulfonate (Intermediate Q3, 100 mg, 0.276 mmol), N,N-diisopropylethylamine (0.145 ml, 0.828 mmol), Xantphos (35 mg, 0.061 mmol), tris(dibenzylideneacetone)dipalladium (0) (28 mg, 0.03 mmol) and methyl 3-mercapto-[1,2,4]triazolo[4,3-b]pyridazine-6-carboxylate (Intermediate I, 58 mg, 0.276 mmol) in DMF (1 ml) was degased by bubbling in N₂ for 2 min at rt. The reaction mixture was stirred at 70° C. for 30 min. After cooling to rt, 1-(3-dimethylaminopropyl)-3-ethylycarbodiimide hydrochloride (106 mg, 0.552 mmol), 1-hydrooxybenzotriazole hydrate (85 mg, 0.552 mmol), N,N-diisopropylethylamine (145 μL, 0.828 mmol) and N,O-dimethylhydroxylamine (55 mg, 0.552 mmol) were added. The reaction mixture was stirred at rt for 12 h, quenched with NaHCO₃ aqueous solution and extracted with CH₂Cl₂. The combined organic layers were concentrated, purified via biotage by flash chromatography on silica gel using a gradient of 0-10% MeOH/CH₂Cl₂ to give the title compound 10.1 (27 mg, 0.061 mmol, 22.0% yield) as a yellow solid. LCMS (method A): [MH]⁺=452, t_{R}=2.14 min.

### 1-(3-((3-morpholin-4-yl-quinolin-6-yl)sulfanyl)-[1,2,4]triazolo[4,3-b]pyridazin-6-yl)ethanone (10.2)

To a solution of 10.1 (27 mg, 0.06 mmol, not complete pure) in THF (1.0 mL) was added methylmagnesium bromide (0.04 mL, 0.12 mmol) solution carefully under N₂ protection at 0° C. The reaction mixture was gradually warmed to rt and kept stirred for 4 h, quenched with NH₄Cl aqueous solution and extracted with CH₂Cl₂. The combined organic layers was concentrated, purified via Biotage by flash chromatography on silica gel using a gradient of 0-3% MeOH/CH₂Cl₂ to afford the title compound 10.2 (10 mg, 0.025 mmol, 41% yield) as a yellow solid. LCMS (method A): [MH]⁺=407, t_{R}=2.27 min.

### (E)-1-[3-(3-Morpholin-4-yl-quinolin-6-ylsulfanyl)-[1,2,4]triazolo[4,3-b]pyridazin-6-yl]-ethanone oxime (Example 10)

To the solution of 10.2 (10 mg, 0.025 mmol) in MeOH (1 ml) was added hydroxylamine hydrochloride (1.71 mg, 0.025 mmol). The reaction mixture was stirred for 5 hr at 60° C., evaporated the solvent and collect the title compound as hydrochloride salt (10.2 mg, 0.024 mmol, 98% yield) as a yellow solid. ¹H-NMR (400 MHz, DMSO) δ ppm 12.3 (s, 1H), 8.94 (m, 1H), 8.39 (d, 1H), 7.92 (d, 1H), 7.87 (m, 2H), 7.72 (m, 1H), 7.56 (m, 1H), 3.77 (m, 4H), 3.32 (m, 4H), 2.03 (s, 3H). LCMS (method A): [MH]⁺=422, t_{R}=2.36 min.

### Example 11

### Method 1B

### (E)-1-[3-(5,7-Difluoro-3-morpholin-4-yl-quinolin-6-ylmethyl)-[1,2,4]triazolo[4,3-b]pyridazin-6-yl]-ethanone oxime

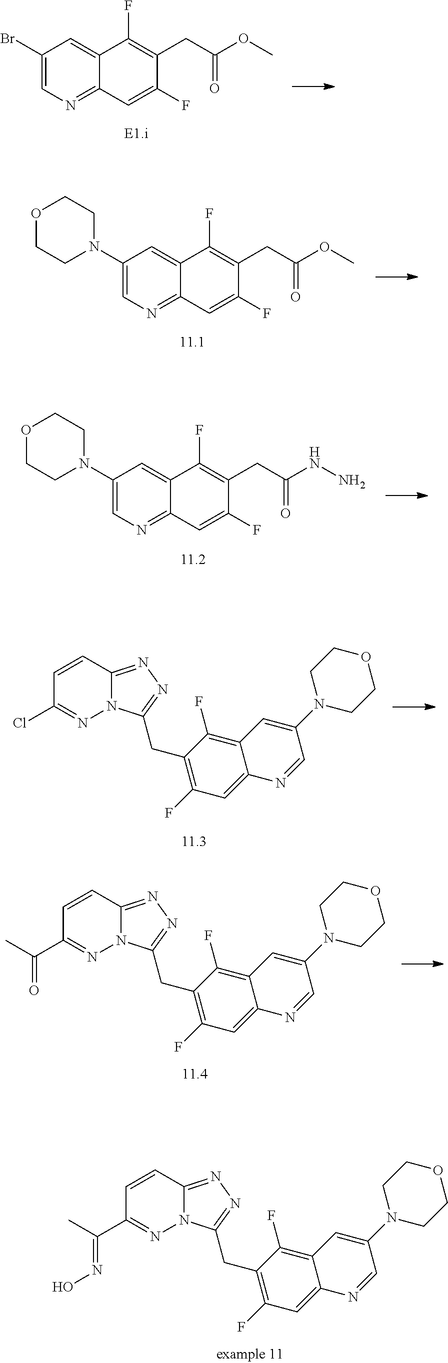

### Methyl 2-(5,7-difluoro-3-morpholin-4-yl-quinolin-6-yl)acetate (11.1)

A solution of methyl 2-(3-bromo-5,7-difluoroquinolin-6-yl)acetate (E1.i) (1.0 g, 3.16 mmol) and morpholine (469 mg, 5.38 mmol) in toluene (20 mL) was purged with argon for 3 min, followed by addition of Pd₂(dba)₃ (290 mg, 0.316 mmol), BINAP (591 mg, 0.949 mmol) and t-BuONa (426 mg, 4.43 mmol) sequentially. The mixture was purged with argon for another half min. The reaction mixture was stirred at 110° C. for 5 h under argon. Then the reaction mixture was cooled to rt, water was added and the product was then extracted with EtOAc. The organic layers were combined, dried over Na₂SO₄ and concentrated under reduced pressure. The residue was purified by column chromatography (33% EtOAc in hexane) to afford 195 mg (19%) of the title compound 11.1. ¹H-NMR (400 MHz, CDCl₃) δ ppm 8.81 (s, 1H), 7.69 (d, 1H), 7.62 (s, 1H), 3.95-3.93 (m, 4H), 3.90 (s, 2H), 3.75 (s, 3H), 3.33-3.31 (m, 4H). LCMS (method A): [MH]⁺=323, t_{R}=2.37 min.

### 2-(5,7-Difluoro-3-morpholin-4-yl-quinolin-6-yl)acetohydrazide (11.2)

To a solution of 11.1 (195 mg, 0.605 mmol) in methanol (5 mL) was added hydrazine monohydrate (1 mL, 20 mmol), and the reaction mixture was stirred at reflux for 0.5 h.

Solvent was removed under reduced pressure, and the residue (11.2) was used without further purification. LCMS (method A): [MI-1]⁺=323, t_{R}=1.742 min.

### 4-(6-((6-Chloro-[1,2,4]triazolo[4,3-b]pyridazin-3-yl)methyl)-5,7-difluoroquinolin-3-yl)morpholine (11.3)

A solution of 11.2 (130 mg, 0.403 mmol) and 3,6-dichloropyridazine (72.1 mg, 0.484 mmol) in butan-1-ol (5 mL) was stirred at 140° C. under microwave irradiation for 6 h. Solvent was removed under reduced pressure, and the residue was purified by column chromatography (10% methanol in ethyl acetate) to afford 132 mg (79%) of the title compound 11.3 as a brown solid. ¹H-NMR (400 MHz, DMSO-d₆) δ ppm 9.00 (s, 1H), 8.45 (d, 1H), 7.61 (d, 1H), 7.53-7.49 (m, 2H), 4.65 (s, 2H), 3.80-3.79 (m, 4H), 3.34-3.31 (m, 4H). LCMS (method A): [MH]⁺=417, t_{R}=2.387 min.

### 1-(3-((5,7-Difluoro-3-morpholin-4-yl-quinolin-6-yl)methyl)-[1,2,4]triazolo[4,3-b]pyridazin-6-yl)ethanone (11.4)

A solution of 11.3 (130 mg, 0.312 mmol) in 1,4-dioxane (10 mL) was purged with argon for 3 min, followed by addition of PdCl₂(PPh₃)₂ (22 mg, 0.031 mmol) and tributyl-(1-ethoxy-vinyl)-stannane (225 mg, 0.624 mmol) sequentially. The mixture was purged with argon for another half min. The reaction mixture was stirred at 110° C. for 2 h under argon. Then the reaction mixture was cooled to rt, 3 N HCl was added and the mixture was stirred for additional 16 h. Water was added, neutralized with NaHCO₃ aqueous solution, and the product was then extracted with dichloromethane. The organic layers were combined, dried over Na₂SO₄ and concentrated under reduced pressure. The residue was purified by column chromatography (10% MeOH in ethyl acetate) to afford 60 mg (45%) of the title compound 11.4. LCMS (method A): [MH]⁺=425, t_{R}=2.10 min.

### (E)-1-(3-((5,7-Difluoro-3-morpholin-4-yl-quinolin-6-yl)methyl)-[1,2,4]triazolo[4,3-b]pyridazin-6-yl)ethanone oxime (Example 11)

A solution of 11.4 (60 mg, 0.141 mmol) and hydroxylamine hydrochloride (29.5 mg, 0.424 mmol) in methanol (5 mL) and HCl (4 N in 1,4-dioxane, 0.1 mL) was stirred at 45° C. for 3 h. The solvent was removed under reduced pressure and the residue was diluted with dichloromethane, neutralized with NaHCO₃ aqueous solution, the product was extracted with dichloromethane. The organic layers were combined, dried over Na₂SO₄ and concentrated under reduced pressure. The residue was purified by column chromatography (10% MeOH in dichloromethane) to afford 33 mg (53%) of the title compound. ¹H-NMR (400 MHz, DMSO-d₆) δ ppm 12.30 (s, 1H), 8.97 (d, 1H), 8.23 (d, 1H), 7.73 (d, 1H), 7.58 (d, 1H), 7.46 (d, 1H), 4.72 (s, 2H), 3.80-3.78 (m, 4H), 3.30-3.29 (m, 4H), 2.16 (s, 3H). LCMS (method A): [MH]⁺=440, t_{R}=2.25 min.

### Example 12

### (E)-1-(3-((3-(1-Methyl-1H-pyrazol-4-yl)quinolin-6-yl)methyl)-[1,2,4]triazolo[4,3-b]pyridazin-6-Methanone oxime

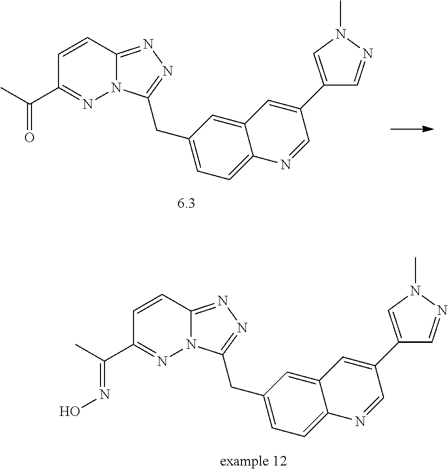 [SMILES: CC(=O)c1ccc2nnc(Cc3ccc4ncc(-c5cnn(C)c5)cc4c3)n2n1 ; C/C(=N\O)c1ccc2nnc(Cc3ccc4ncc(-c5cnn(C)c5)cc4c3)n2n1]

### (E)-1-(3-((3-(1-Methyl-1H-pyrazol-4-yl)quinolin-6-yl)methyl)-[1,2,4]triazolo[4,3-b]pyridazin-6-yl)ethanone oxime (Example 12)

The title compound was prepared from 1-{3-[3-(1-methyl-1H-pyrazol-4-yl)quinolin-6-ylmethyl]-[1,2,4]triazolo[4,3-b]pyridazin-6-yl}-ethanone (6.3) using the same procedure as described in the synthesis of Example 11. ¹H-NMR (400 MHz, DMSO-d₆) δ ppm 12.27 (s, 1H), 9.12 (s, 1H), 8.38 (d, 2H), 8.25 (d, 1H), 8.07 (s, 1H), 7.93 (d, 1H), 7.83 (s, 1H), 7.75-7.69 (m, 2H), 4.74 (s, 2H), 3.90 (s, 3H), 2.22 (s, 3H). LCMS (method A): [MH]⁺=399, t_{R}=2.025 min.

### Example 13

### (E)-1-(3-((3-Morpholin-4-yl-quinolin-6-yl)methyl)-[1,2,4]triazolo[4,3-b]pyridazin-6-yl)ethanone oxime

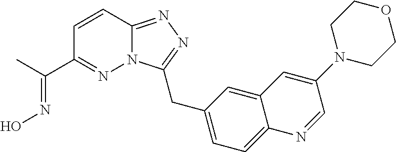 [SMILES: C/C(=N\O)c1ccc2nnc(Cc3ccc4ncc(N5CCOCC5)cc4c3)n2n1]

The title compound was prepared using the same procedure as described in the synthesis of Example 11 by using Intermediate C1 instead of (11.1). ¹H-NMR (400 MHz, DMSO-d₆) δ ppm 12.29 (s, 1H), 8.81 (d, 1H), 8.24 (d, 1H), 7.80 (d, 1H), 7.73 (d, 1H), 7.70 (s, 1H), 7.51 (dd, 1H), 7.46 (d, 1H), 4.68 (s, 2H), 3.78 (t, 4H), 3.24 (t, 4H), 2.21 (s, 3H). LCMS (method A): [MH]⁺=404, t_{R}=2.026 min.

### Example 14

### (E)-1-(3-((3-(4-Methylpiperazin-1-yl)quinolin-6-yl)methyl)-[1,2,4]triazolo[4,3-b]pyridazin-6-yl)ethanone oxime

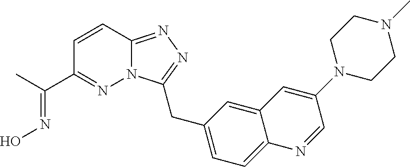 [SMILES: C/C(=N\O)c1ccc2nnc(Cc3ccc4ncc(N5CCN(C)CC5)cc4c3)n2n1]

The title compound was prepared using the same procedure as described in the synthesis of Example 11 by using an equivalent amount of Intermediate C1 instead of (11.1). ¹H-NMR (400 MHz, DMSO-d₆) δ ppm 12.25 (s, 1H), 8.79 (s, 1H), 8.24 (d, 1H), 7.79 (d, 1H), 7.73 (d, 1H), 7.68 (s, 1H), 7.49 (d, 1H), 7.44 (s, 1H), 4.67 (s, 2H), 3.30 (s, 4H), 2.49 (m, 4H), 2.23 (s, 3H), 2.21 (s, 3H). LCMS (method A): [MH]⁺=417, t_{R}=1.276 min.

### Example 15

### (E)-1-(3-((3-(Morpholin-4-yl-methyl)quinolin-6-yl)methyl)-[1,2,4]triazolo[4,3-b]pyridazin-6-yl)ethanone oxime

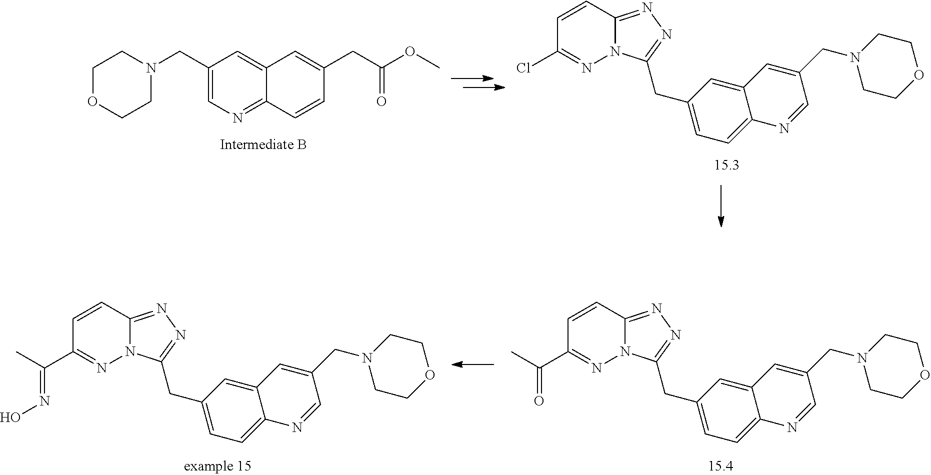 [SMILES: COC(=O)Cc1ccc2ncc(CN3CCOCC3)cc2c1 ; Clc1ccc2nnc(Cc3ccc4ncc(CN5CCOCC5)cc4c3)n2n1 ; C/C(=N\O)c1ccc2nnc(Cc3ccc4ncc(CN5CCOCC5)cc4c3)n2n1 ; CC(=O)c1ccc2nnc(Cc3ccc4ncc(CN5CCOCC5)cc4c3)n2n1]

### 4-((6-((6-Chloro-[1,2,4]triazolo[4,3-b]pyridazin-3-yl)methyl)quinolin-3-yl)methyl)morpholine (15.3) was prepared from methyl 2-(3-(morpholin-4-yl-methyl)quinolin-6-yl)acetate (Intermediate B) using the same procedure as described in the synthesis of 11.2 and 11.3. LCMS (method A): [MH]⁺=395, t_{R}=1.233 min.

1-(3-((3-(Morpholin-4-yl-methyl)quinolin-6-yl)methyl)-[1,2,4]triazolo[4,3-b]pyridazin-6-yl)ethanone (15.4) was prepared from 15.3 using the same procedure as described in the synthesis of 11.4. LCMS (method A): [MH]⁺=403, t_{R}=0.262 min.

(E)-1-(3-((3-(Morpholin-4-yl-methyl)quinolin-6-yl)methyl)-[1,2,4]triazolo[4,3-b]pyridazin-6-yl)ethanone oxime (Example 15) was prepared using the same procedure as described in the synthesis of Example 11. ¹H-NMR (400 MHz, CDCl₃) δ ppm 9.44 (s, 1H), 8.88 (s, 1H), 8.07 (d, 2H), 7.96 (d, 1H), 7.87 (s, 1H), 7.82 (d, 1H), 7.75 (d, 1H), 4.79 (s, 2H), 3.83-3.74 (m, 6H), 2.55 (s_{b}, 4H), 2.36 (s, 3H). LCMS (method A): [MH]⁺=418, t_{R}=1.36 min.

### Example 16

### (E)-1-(3-((3-((4-Methylpiperazin-1-yl)methyl)quinolin-6-yl)methyl)-[1,2,4]triazolo[4,3-b]pyridazin-6-yl)ethanone oxime

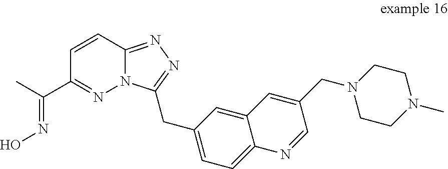 [SMILES: C/C(=N\O)c1ccc2nnc(Cc3ccc4ncc(CN5CCN(C)CC5)cc4c3)n2n1]

The title compound was prepared using the same procedure as described in the synthesis of Example 15 by starting from [3-(4-Methyl-piperazin-1-ylmethyl)-quinolin-6-yl]-acetic acid methyl ester (Intermediate B1). ¹H-NMR (400 MHz, CDCl₃) δ ppm 12.56 (s_{b}, 1H), 8.87 (s, 1H), 8.05 (d, 1H), 7.97 (s, 1H), 7.92 (d, 1H), 7.83-7.74 (m, 3H), 4.78 (s, 2H), 3.70 (s, 2H), 2.58 (s_{b}, 8H), 2.35 (s, 6H). LCMS (method A): [MH]⁺=431, t_{R}=1.27 min.

### Example 17

### (E)-1-(3-((5,7-Difluoro-3-(morpholin-4-yl-methyl)quinolin-6-yl)methyl)-[1,2,4]triazolo[4,3-b]pyridazin-6-yl)ethanone oxime

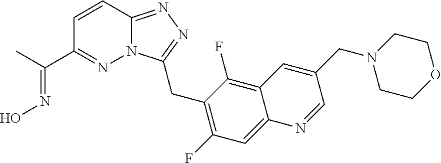 [SMILES: C/C(=N\O)c1ccc2nnc(Cc3c(F)cc4ncc(CN5CCOCC5)cc4c3F)n2n1]

The title compound was prepared using the same procedure as described in the synthesis of Example 15 by starting from (5,7-difluoro-3-morpholin-4-ylmethyl-quinolin-6-yl)-acetic acid methyl ester (Intermediate B2). ¹H-NMR (400 MHz, DMSO-d₆) δ ppm 12.28 (s, 1H), 8.93 (s, 1H), 8.32 (s, 1H), 8.23 (d, 1H), 7.74-7.71 (m, 2H), 4.75 (s, 2H), 3.70 (s, 2H), 3.57 (s_{b}, 4H), 2.40 (s_{b}, 4H), 2.14 (s, 3H). LCMS (method A): [MH]⁺=454, t_{R}=1.523 min.

### Example 18

### (E)-1-(3-((3-(Piperidin-1-ylmethyl)quinolin-6-yl)methyl)-[1,2,4]triazolo[4,3-b]pyridazin-6-yl)ethanone oxime

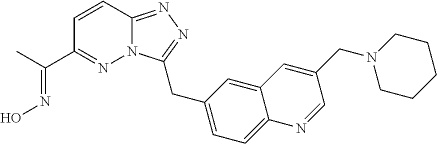 [SMILES: C/C(=N\O)c1ccc2nnc(Cc3ccc4ncc(CN5CCCCC5)cc4c3)n2n1]

The title compound was prepared using the same procedure as described in the synthesis of Example 15 by starting from (3-(piperidin-1-ylmethyl)quinolin-6-yl)-acetic acid methyl ester (Intermediate B3). ¹H-NMR (400 MHz, CDCl₃) δ ppm 11.82 (s, 1H), 8.84 (d, 1H), 8.05 (d, 2H), 7.90-7.79 (m, 3H), 7.70 (d, 1H), 4.76 (s, 2H), 3.69 (s, 2H), 2.48 (s_{b}, 4H), 2.35 (s, 3H), 1.63 (s_{b}, 4H), 1.47 (s, 2H). LCMS (method A): [MH]⁺=416, t_{R}=1.624 min.

### Example 19

### (E)-1-(3-((3-((1S,4S)-5-Methyl-2,5-diazabicyclo[2.2.1]heptan-2-yl)quinolin-6-yl)methyl)-[1,2,4]triazolo[4,3-b]pyridazin-6-yl)ethanone oxime

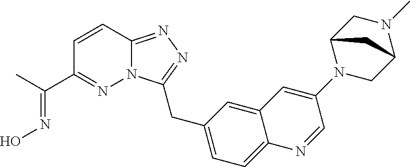 [SMILES: C/C(=N\O)c1ccc2nnc(Cc3ccc4ncc(N5C[C@@H]6C[C@H]5CN6C)cc4c3)n2n1]

The title compound was prepared using the same procedure as described in the synthesis of Example 15 by starting from 2-(3-((1S,4S)-5-methyl-2,5-diazabicyclo[2.2.1]heptan-2-yl)quinolin-6-yl)acetic acid methyl ester (Intermediate C). ¹H-NMR (400 MHz, CDCl₃) δ ppm 8.46 (s, 1H), 7.85 (t, 2H), 7.59-7.57 (m, 2H), 7.48 (d, 1H), 6.90 (s, 1H), 4.68 (s, 2H), 4.40 (s, 1H), 3.60-3.55 (m, 3H), 3.46-3.44 (m, 1H), 2.91-2.83 (m, 2H), 2.43 (s, 3H), 2.29 (s, 3H), 1.99-1.97 (m, 2H). LCMS (method A): [MH]⁺=429, t_{R}=1.570 min.

Deviating from the procedure in Example 15, here, intermediate compound 6-((6-Chloro-[1,2,4]triazolo[4,3-b]pyridazin-3-yl)methyl)-3-((1S,4S)-5-methyl-2,5-diazabicyclo[2.2.1]heptan-2-yl)quinoline was prepared by stirring a solution of 2-(3-((1S,4S)-5-methyl-2,5-diazabicyclo[2.2.1]heptan-2-yl)quinolin-6-yl)acetohydrazide (320 mg, 1.028 mmol) and 3,6-dichloropyridazine (306 mg, 2.055 mmol) in butan-1-ol (10 mL) at 180° C. under microwave irradiation for 7 h. Solvent was removed under reduced pressure, and the residue was purified by column chromatography (20% methanol in dichloromethane) to afford 284 mg (68%) of the respective compound (LCMS (method A): [M H]⁺=406, t_{R}=1.828 min).

### Example 20

### (E)-1-(3-((3-(4-Hydroxypiperidin-1-yl)quinolin-6-yl)methyl)-[1,2,4]triazolo[4,3-b]pyridazin-6-yl)ethanone oxime

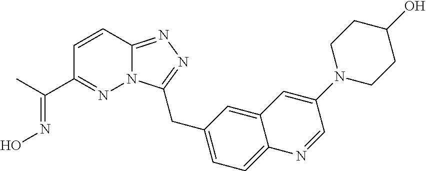 [SMILES: C/C(=N\O)c1ccc2nnc(Cc3ccc4ncc(N5CCC(O)CC5)cc4c3)n2n1]

The title compound was prepared using the same procedure as described in the synthesis of Example 15 by starting from methyl 2-(3-(4-hydroxypiperidin-1-yl)quinolin-6-yl)acetate (Intermediate D). ¹H-NMR (400 MHz, DMSO-d₆) δ ppm 8.77 (s, 1H), 8.19 (d, 1H), 7.77 (d, 2H), 7.67 (s, 1H), 7.48-7.43 (m, 2H), 4.65 (s, 2H), 3.66-3.63 (m, 3H), 2.98 (t, 2H), 2.19 (s, 3H), 1.86-1.83 (m, 2H), 1.54-1.49 (m, 2H). LCMS (method A): [MH]⁺=418, t_{R}=1.970 min.

Deviating from the procedure in Example 15, here, intermediate compound 1464(6-chloro-[1,2,4]triazolo[4,3-b]pyridazin-3-yl)methyl)quinolin-3-yl)piperidin-4-ol was prepared by stirring a solution of 2-(3-(4-hydroxypiperidin-1-yl)quinolin-6-yl)acetohydrazide (290 mg, 0.966 mmol) and 3,6-dichloropyridazine (288 mg, 1.931 mmol) in butan-1-ol (10 mL) at 180° C. under microwave irradiation for 1 h. Solvent was removed under reduced pressure, and the residue was purified by column chromatography (10% methanol in ethyl acetate) to afford 230 mg (60%) of the respective compound. LCMS (method A): [MH]⁺=395, t_{R}=1.879 min.

### Example 21-(S) and Example 21-(R)

### (E)-1-(3-(1-(5,7-Difluoro-3-(1-methyl-1H-pyrazol-4-yl)quinolin-6-yl)ethyl)-[1,2,4]triazolo[4,3-b]pyridazin-6-yl)ethanone oxime

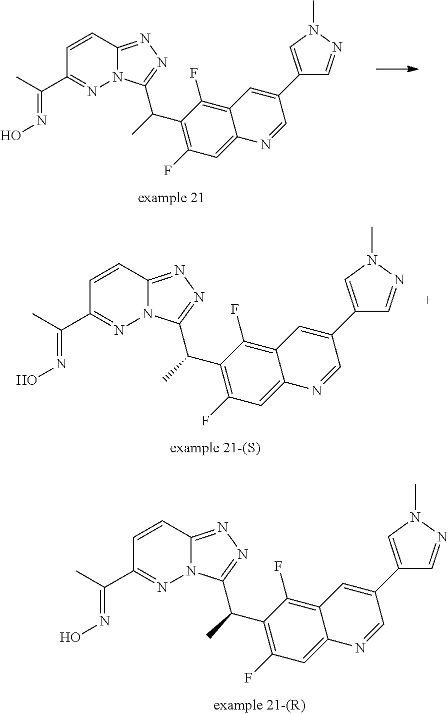 [SMILES: C/C(=N\O)c1ccc2nnc(C(C)c3c(F)cc4ncc(-c5cnn(C)c5)cc4c3F)n2n1 ; C/C(=N\O)c1ccc2nnc([C@@H](C)c3c(F)cc4ncc(-c5cnn(C)c5)cc4c3F)n2n1 ; C/C(=N\O)c1ccc2nnc([C@H](C)c3c(F)cc4ncc(-c5cnn(C)c5)cc4c3F)n2n1]

The title compound was prepared using the same procedure as described in the synthesis of Example 1 by starting from methyl 2-(5,7-difluoro-3-(1-methyl-1H-pyrazol-4-yl)quinolin-6-yl)propanoate (Intermediate E). The enantiomers were obtained by separation of racemic mixture (E)-1-(3-(1-(5,7-difluoro-3-(1-methyl-1H-pyrazol-4-yl)quinolin-6-yl)ethyl)-[1,2,4]triazolo[4,3-b]pyridazin-6-yl)ethanone oxime (Example 21, 90 mg, 0.201 mmol) by preparatory SFC (method C). Data for (S,E)-1-(3-(1-(5,7-difluoro-3-(1-methyl-1H-pyrazol-4-yl)quinolin-6-yl)ethyl)-[1,2,4]triazolo[4,3-b]pyridazin-6-yl)ethanone oxime (21, 10.1 mg): ¹H-NMR (400 MHz, DMSO-d₆) 8 ppm 12.22 (s, 1H), 9.24 (s, 1H), 8.46 (d, 2H), 8.22 (d, 1H), 8.15 (s, 1H), 7.63-7.67 (m, 2H), 5.24 (q, 1H), 3.89 (s, 3H), 2.02 (d, 3H), 1.86 (s, 3H). LCMS (method A): [MH]⁺=449, t_{R}=2.32 min. Data for (R,E)-1-(3-(1-(5,7-difluoro-3-(1-methyl-1H-pyrazol-4-yl)quinolin-6-yl)ethyl)-[1,2,4]triazolo[4,3-b]pyridazin-6-yl)ethanone oxime (21*, 10.3 mg): ¹H-NMR (400 MHz, DMSO-d₆) 8 ppm 12.22 (s, 1H), 9.24 (s, 1H), 8.46 (d, 2H), 8.22 (d, 1H), 8.15 (s, 1H), 7.63-7.67 (m, 2H), 5.24 (q, 1H), 3.89 (s, 3H), 2.02 (d, 3H), 1.86 (s, 3H). LCMS (method A): [MH]⁺=449, t_{R}=2.32 min.

### Example 22

### (E)-1-(3-((5,7-Difluoro-3-(1-methyl-1H-pyrazol-4-yl)quinolin-6-yl)methyl)-[1,2,4]triazolo[4,3-b]pyridazin-6-yl)ethanone oxime

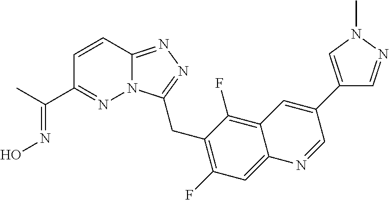 [SMILES: C/C(=N\O)c1ccc2nnc(Cc3c(F)cc4ncc(-c5cnn(C)c5)cc4c3F)n2n1]

The title compound was prepared using the same procedure as described in the synthesis of Example 1 by starting from methyl 2-(5,7-difluoro-3-(1-methyl-1H-pyrazol-4-yl)quinolin-6-yl)acetate (Intermediate E1). ¹H-NMR (400 MHz, DMSO-d₆) 8 ppm 12.37 (s br, 1H), 9.27 (s, 1H), 8.53 (s, 1H), 8.48 (s, 1H), 8.24 (d, 1H), 8.18 (s, 1H), 7.68-7.76 (m, 2H), 4.76 (s, 2H), 3.90 (s, 3H), 2.16 (s, 3H). LCMS (method A): [MH]⁺=435, t_{R}=2.21 min.

### Example 23

### (E)-1-(3-((3-(3,5-Dimethylisoxazol-4-yl)-5,7-difluoroquinolin-6-yl)methyl)-[1,2,4]triazolo[4,3-b]pyridazin-6-yl)ethanone oxime

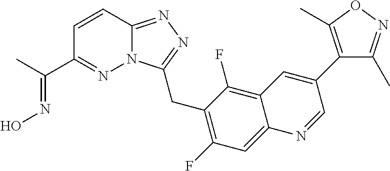 [SMILES: C/C(=N\O)c1ccc2nnc(Cc3c(F)cc4ncc(-c5c(C)noc5C)cc4c3F)n2n1]

The title compound was prepared using the same procedure as described in the synthesis of Example 1 by starting from methyl 2-(3-(3,5-dimethylisoxazol-4-yl)-5,7-difluoroquinolin-6-yl)acetate (Intermediate F). ¹H-NMR (400 MHz, DMSO-d₆) 8 ppm 12.35 (s br, 1H), 9.02 (s, 1H), 8.49 (s, 1H), 8.25 (d, 1H), 7.73-7.81 (m, 2H), 4.78 (s, 2H), 2.49 (s, 3H), 2.29 (s, 3H), 2.16 (s, 3H). LCMS (method A): [MH]⁺=450, t_{R}=2.47 min.

### Example 24, 24-(R) and 24-(S)

### (E)-1-(3-(1-(5,7-Difluoro-3-(2-hydroxypropan-2-yl)quinolin-6-yl)ethyl)-[1,2,4]triazolo[4,3-b]pyridazin-6-yl)ethanone oxime

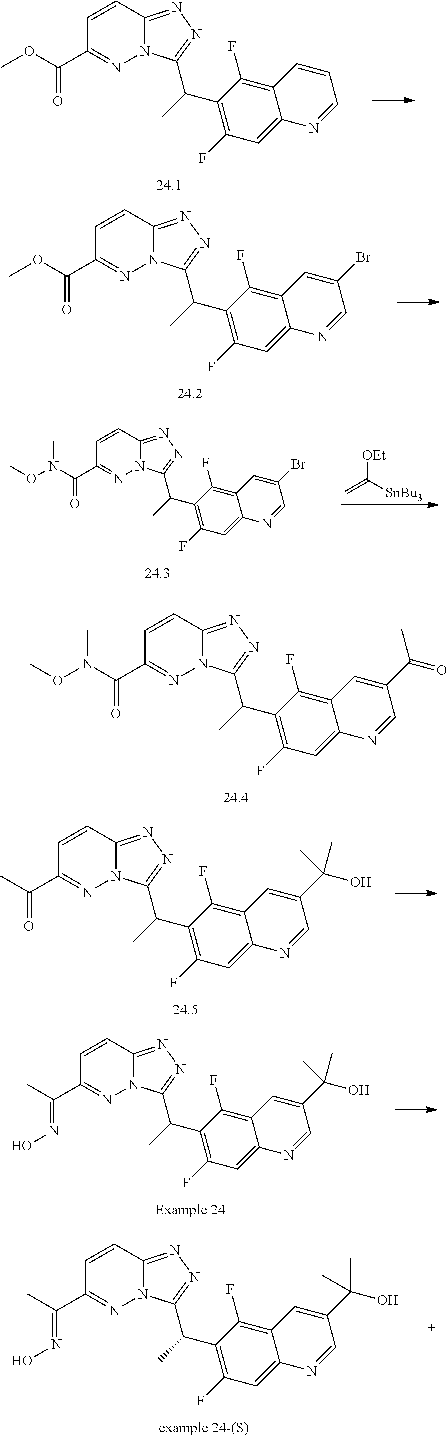

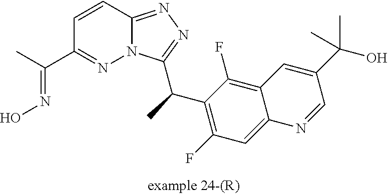 [SMILES: C/C(=N\O)c1ccc2nnc([C@H](C)c3c(F)cc4ncc(C(C)(C)O)cc4c3F)n2n1]

### Methyl 3-(1-(3-bromo-5,7-difluoroquinolin-6-yl)ethyl)-[1,2,4]triazolo[4,3-b]pyridazine-6-carboxylate (24.2)

Intermediate 24.1 was produced using the same procedure for intermediate 9.2. To a solution of methyl 3-(1-(5,7-difluoroquinolin-6-yl)ethyl)-[1,2,4]triazolo[4,3-b]pyridazine-6-carboxylate (24.1, 750 mg, 2.031 mmol—obtained in analogy to compound 9.2 in Example 9 starting from 5,7-difluoro-2-quinolin-6-yl-propionic acid) in CCl₄ (20 mL) was added bromine (0.21 mL, 4.06 mmol) at rt, and the reaction mixture was heated to reflux. The reaction was cooled to rt, and pyridine (0.41 mL, 5.08 mmol) was added dropwise. The reaction was heated to reflux for 2 h. The mixture was diluted with CH₂Cl₂, neutralized with satd. aqueous NaHCO₃ solution, extracted, dried, concentrated, and purified by column chromatography to afford 700 mg of the title compound as white solid. ¹H-NMR (400 MHz, CDCl₃) 8 ppm 8.93 (s, 1H), 8.53 (s, 1H), 8.20 (d, 1H), 7.72 (d, 1H), 7.63 (d, 1H), 5.34 (q, 1H), 3.94 (s, 3H), 2.18 (d, 3H). LCMS (method A): [MH]⁺=449, t_{R}=2.52 min.

### 3-(1-(3-Bromo-5,7-difluoroquinolin-6-yl)ethyl)-N-methoxy-N-methyl-[1,2,4]triazolo[4,3-b]pyridazine-6-carboxamide (24.3)

To a solution of (24.2) (700 mg, 1.09 mmol) in THF (20 mL) was added LiOH (100 mg, 4.18 mmol) followed by water (2 mL), and the reaction mixture was stirred at rt overnight. LCMS showed most satarting material was consumed. N,O-dimethylhydroxylamine hydrochloride (200 mg, 2.05 mmol), N-methylmorpholine (0.25 mL, 2.27 mmol), and HATU (1.00 g, 2.63 mmol) was added successively, and the reaction mixture was stirred at rt for 5 h. Aqueous K₂CO₃ solution was added, and the reaction mixture was extracted with methylene chloride, dried, concentrated, and purified by column chromatography followed by HPLC to afford 404 mg of the title compound 24.3 as white solid. ¹H-NMR (400 MHz, DMSO-d₆) 8 ppm 9.03 (s, 1H), 8.83 (s, 1H), 8.46 (d, 1H), 7.76 (d, 1H), 7.44 (d, 1H), 5.26 (q, 1H), 3.16 (s, 3H), 3.15 (s, 3H), 1.98 (d, 3H). LCMS (method A): [MH]⁺=478, t_{R}=2.34 min.

### 3-(1-(3-Acetyl-5,7-difluoroquinolin-6-yl)ethyl)-N-methoxy-N-methyl-[1,2,4]triazolo[4,3-b]pyridazine-6-carboxamide (24.4)

24.3 (100 mg, 0.21 mmol) in dioxane (10 mL) was bubbled with argon for 3 min, followed by addition of tributyl(1-ethoxyvinyl)stannane (114 mg, 0.314 mmol), and PdCl₂(PPh₃)₂ (14.7 mg, 0.021 mmol). The reaction mixture was heated at 80° C. for 3 h. The reaction mixture was diluted with EtOAc, washed with KF aqueous solution and brine, dried, and concentrated to use without further purification. The crude reside was diluted with methanol (10 mL), and 3N HCl (2 mL) was added, and the reaction mixture was stirred at rt for 2 h. LCMS showed the reaction was complete. The reaction mixture was purified by HPLC to afford 50 mg of the title compound 24.4 as yellow syrup. LCMS (method A): [MH]⁺=441, t_{R}=2.03 min.

### 1-(3-(1-(5,7-Difluoro-3-(2-hydroxypropan-2-yl)quinolin-6-yl)ethyl)-[1,2,4]triazolo[4,3-b]pyridazin-6-yl)ethanone (24.5)

To 24.4 (50 mg, 0.114 mmol) in THF (15 mL) was added methylmagnesium iodide (3N in THF, 0.378 mL, 1.135 mmol) at −78° C., and the reaction mixture was stirred for 15 min, and the reaction mixture was allowed to rise to 0° C. naturally. The reaction was quenched with satd. NH₄Cl aqueous solution, extracted with EtOAc, dried, concentrated, and purified by column chromatography to afford 28 mg of the title compound 24.5 as yellow syrup. LCMS (method A): [MH]⁺=412, t_{R}=2.22 min.

### (E)-1-(3-(1-(5,7-Difluoro-3-(2-hydroxypropan-2-yl)quinolin-6-yl)ethyl)-[1,2,4]triazolo[4,3-b]pyridazin-6-yl)ethanone oxime (Example 24)

To 24.5 (50 mg, 0.122 mmol) in MeOH (5 mL) was added hydroxylamine hydrochloride (40 mg, 0.576 mmol), and the reaction mixture was stirred at rt overnight. LCMS showed the reaction was complete, the mixture was tuned with 1N NaOH until pH 8-9, concentrated, and purified by column chromatography to afford 30 mg of the title compound as white solid. ¹H-NMR (400 MHz, DMSO-d₆) 8 ppm 12.22 (s, 1H), 9.10 (s, 1H), 8.34 (s, 1H), 8.22 (d, 1H), 7.64-7.67 (m, 2H), 5.43 (s, 1H), 5.24 (q, 1H), 2.02 (d, 3H), 1.85 (s, 3H), 1.52 (s, 6H). LCMS (method A): [MH]⁺=427, t_{R}=2.27 min.

Separation of the racemic mixture 24 by preparator SFC using the method C provided 24S(S-isomer) and 24 R(R-isomer).

### Example 25

### (E)-1-(3-((3-Cyclohexyl-5,7-difluoroquinolin-6-yl)methyl)-[1,2,4]triazolo[4,3-b]pyridazin-6-yl)ethanone O-2-hydroxyethyl oxime

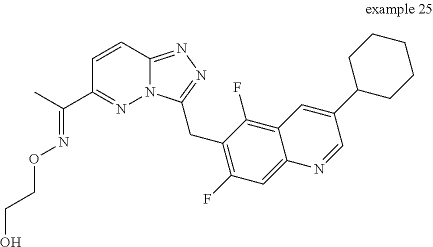 [SMILES: C/C(=N\OCCO)c1ccc2nnc(Cc3c(F)cc4ncc(C5CCCCC5)cc4c3F)n2n1]

The title compound was prepared using the same procedure as described in the synthesis of Example 15 by starting from methyl 2-(3-cyclohexyl-5,7-difluoroquinolin-6-yl)acetate (Intermediate G) and by using the equivalent amount of O-ethylhydroxylamine instead of the hydroxylamine. ¹H-NMR (400 MHz, CDCl₃) δ ppm 8.86 (s, 1H), 8.26 (s, 1H), 8.01 (d, 1H), 7.79 (d, 2H), 4.82 (s, 2H), 4.44-4.42 (m, 2H), 3.98-3.97 (m, 2H), 2.79 (t, 1H), 2.33 (s, 3H), 2.01-1.93 (m, 5H), 1.55-1.43 (m, 5H). LCMS (method A): [MH]⁺=481, t_{R}=2.673 min.

### Example 26

### (E)-1-(3-((3-Cyclohexyl-5,7-difluoroquinolin-6-yl)methyl)-[1,2,4]triazolo[4,3-b]pyridazin-6-yl)-ethanone oxime

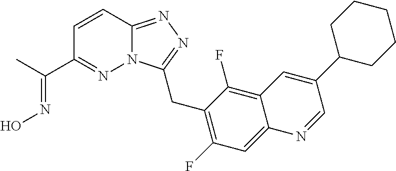 [SMILES: C/C(=N\O)c1ccc2nnc(Cc3c(F)cc4ncc(C5CCCCC5)cc4c3F)n2n1]

The title compound was prepared using the same procedure as described in the synthesis of Example 15 by starting from methyl 2-(3-cyclohexyl-5,7-difluoroquinolin-6-yl)acetate (Intermediate G). ¹H-NMR (400 MHz, CDCl₃) δ ppm 9.28 (s, 1H), 8.85 (s, 1H), 8.21 (s, 1H), 7.99 (d, 1H), 7.79 (d, 1H), 7.72 (d, 1H), 4.83 (s, 2H), 2.76 (t, 1H), 2.33 (s, 3H), 2.00-1.92 (m, 5H), 1.54-1.43 (m, 5H). LCMS (method A): [MH]⁺=437, t_{R}=2.748 min.

### Example 27

### (E)-1-(3-({1-[3-(4-Methyl-piperazin-1-yl)quinolin-6-yl]-cyclopropyl}-[1,2,4]triazolo[4,3-b]-pyridazin-6-yl)-ethanone oxime

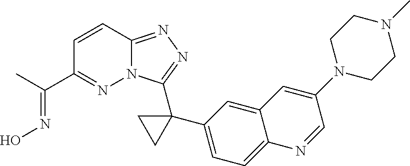 [SMILES: C/C(=N\O)c1ccc2nnc(C3(c4ccc5ncc(N6CCN(C)CC6)cc5c4)CC3)n2n1]

The title compound was prepared using the same procedure as described in the synthesis of Example 15 by starting from 1-[3-(4-methyl-piperazin-1-yl)-quinolin-6-yl]-cyclopropanecarboxylic acid methyl ester (Intermediate C3). ¹H-NMR (400 MHz, CDCl₃) δ ppm 11.95 (s, 1H), 8.73 (d, 1H), 7.89 (dd, 2H), 7.73-7.69 (m, 2H), 7.60 (dd, 1H), 7.24 (d, 1H), 3.36 (s_{b}, 4H), 2.77 (s_{b}, 4H), 2.48 (s, 3H), 2.14 (s, 3H), 1.82-1.80 (m, 2H), 1.64-1.62 (m, 2H). LCMS (method A): [MH]⁺=443, t_{R}=1.740 min.

### Example 28

### (E)-1-(3-((3-(4-Methylpiperazin-1-yl)quinolin-6-yl)sulfanyl)-[1,2,4]triazolo[4,3-b]pyridazin-6-yl)-ethanone oxime

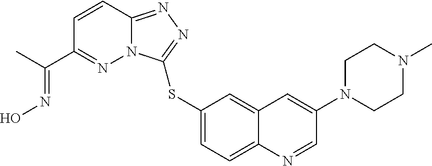 [SMILES: C/C(=N\O)c1ccc2nnc(Sc3ccc4ncc(N5CCN(C)CC5)cc4c3)n2n1]

The title compound was prepared using the same procedure as described in the synthesis of Example 10 by starting from 3-(4-methyl-piperazin-1-yl)-quinolin-6-yl trifluoromethanesulfonate (Intermediate Q4) and methyl 3-mercapto-[1,2,4]triazolo[4,3-b]pyridazine-6-carboxylate (Intermediate I). ¹H-NMR (400 MHz, MeOD) [symbol] ppm 8.80 (d, 1H), 8.17 (d, 1H), 7.93 (m, 3H), 7.86 (m, 1H), 7.59 (d, 1H), 3.59 (m, 4H), 3.29 (m, 4H), 2.83 (s, 3H), 2.08 (s, 3H). LCMS (method B): [M+H]⁺=436, t_{R}=1.78 min.

### Example 29

### (E)-1-(3-((3-(4-Hydroxypiperidin-1-yl)quinolin-6-yl)sulfanyl)-[1,2,4]triazolo[4,3-b]pyridazin-6-yl)-ethanone oxime

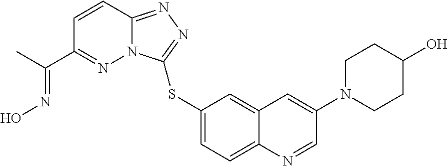 [SMILES: C/C(=N\O)c1ccc2nnc(Sc3ccc4ncc(N5CCC(O)CC5)cc4c3)n2n1]

The title compound was prepared using the same procedure as described in the synthesis of Example 10 by starting from 3-(4-hydroxypiperidin-1-yl)-quinolin-6-yl trifluoromethanesulfonate (Intermediate Q5) and methyl 3-mercapto-[1,2,4]triazolo[4,3-b]pyridazine-6-carboxylate (Intermediate I). ¹H-NMR (400 MHz, MeOD) δ ppm 9.07 (d, 1H), 8.28 (m, 1H), 8.25 (d, 1H), 8.08 (m, 2H), 7.99 (d, 1H), 7.79 (d, 1H), 3.90 (m, 3H), 3.29 (m, 2H), 2.14 (s, 3H), 2.04 (m, 2H), 1.72 (m, 2H). LCMS (method B): [M−H]⁻=434, t_{R}=2.32 min.

### Example 30

### (E)-1-(3-((3-((Tetrahydro-2H-pyran-4-yl)amino)quinolin-6-yl)sulfanyl)-[1,2,4]triazolo[4,3-b]pyridazin-6-yl)-ethanone oxime

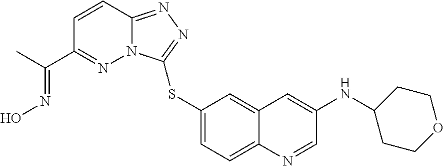 [SMILES: C/C(=N\O)c1ccc2nnc(Sc3ccc4ncc(NC5CCOCC5)cc4c3)n2n1]

The title compound was prepared using the same procedure as described in the synthesis of Example 10 by starting from 3-(tetrahydropyran-4-ylamino)-quinolin-6-yl trifluoromethanesulfonate (Intermediate Q6) and methyl 3-mercapto-[1,2,4]triazolo[4,3-b]pyridazine-6-carboxylate (Intermediate I). ¹H-NMR (400 MHz, DMSO) δ ppm 12.34 (s, 1H), 8.55 (d, 1H), 8.39 (d, 1H), 7.85 (m, 1H), 7.83 (m, 3H), 7.43 (d, 1H), 3.90 (m, 2H), 3.57 (m, 1H), 3.44 (m, 2H), 2.04 (s, 3H), 1.94 (m, 2H), 1.43 (s, 2H). LCMS (method B): [M+H]⁺=437, t_{R}=2.39 min.

### Example 31

### (E)-1-(3-((3-(Morpholin-4-yl-methyl)quinolin-6-yl)sulfanyl)-[1,2,4]triazolo[4,3-b]pyridazin-6-yl)-ethanone oxime

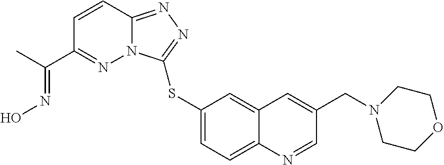 [SMILES: C/C(=N\O)c1ccc2nnc(Sc3ccc4ncc(CN5CCOCC5)cc4c3)n2n1]

The title compound was prepared using the same procedure as described in the synthesis of Example 10 by starting from 3-(morpholin-4-ylmethyl)-quinolin-6-yl trifluoromethanesulfonate (Intermediate Q7) and methyl 3-mercapto-[1,2,4]triazolo[4,3-b]pyridazine-6-carboxylate (Intermediate I). ¹H-NMR (400 MHz, DMSO) δ ppm 9.04 (m, 1H), 8.57 (d, 1H), 8.15 (m, 2H), 8.04 (m, 2H), 7.90 (m, 1H), 4.61 (s, 2H), 3.93 (m, 4H), 3.36 (m, 4H), 2.11 (s, 3H). LCMS (method B): [M+H]⁺=437, t_{R}=1.75 min.

### Example 32

### (E)-1-(3-((3-((diethylamino)methyl)quinolin-6-yl)sulfanyl)-[1,2,4]triazolo[4,3-b]pyridazin-6-yl)ethanone oxime

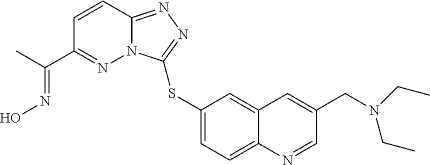 [SMILES: CCN(CC)Cc1cnc2ccc(Sc3nnc4ccc(/C(C)=N/O)nn34)cc2c1]

The title compound was prepared using the same procedure as described in the synthesis of Example 10 by starting from 3-((diethylamino)methyl)quinolin-6-yl trifluoromethanesulfonate (Intermediate Q2) and methyl 3-mercapto-[1,2,4]triazolo[4,3-b]pyridazine-6-carboxylate (Intermediate I). ¹H-NMR (400 MHz, MeOD) [symbol] ppm 9.01 (d, 1H), 8.55 (d, 1H), 8.20 (m, 2H), 8.05 (m, 2H), 7.87 (d, 1H), 4.60 (s, 2H), 3.25 (m, 4H), 2.10 (s, 3H), 1.42 (m, 6H). LCMS (method B): [M+H]⁺=422, t_{R}=1.71 min.

### Example 33

### (E)-1-(3-((3-(3-(dimethylamino)pyrrolidin-1-yl)quinolin-6-yl)sulfanyl)-[1,2,4]triazolo[4,3-b]pyridazin-6-yl)ethanone oxime

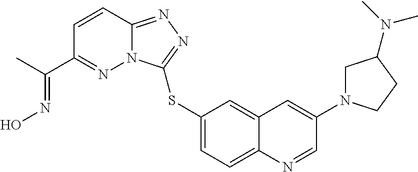 [SMILES: C/C(=N\O)c1ccc2nnc(Sc3ccc4ncc(N5CCC(N(C)C)C5)cc4c3)n2n1]

The title compound was prepared using the same procedure as described in the synthesis of Example 10 by starting from (S)-3-(3-(dimethylamino)pyrrolidin-1-yl)quinolin-6-yl trifluoromethanesulfonate (Intermediate Q1) and methyl 3-mercapto-[1,2,4]triazolo[4,3-b]pyridazine-6-carboxylate (Intermediate I). LCMS (method B): [M+H]⁺=449, t_{R}=1.79 min.

### Example 34

### (E)-1-{3-[3-(Tetrahydro-pyran-4-ylamino)-quinolin-6-ylmethyl]-[1,2,4]triazolo[4,3-b]pyridazin-6-yl}-ethanone oxime

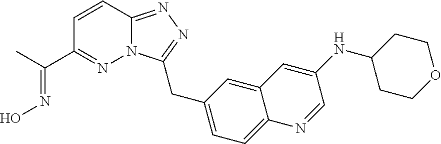 [SMILES: C/C(=N\O)c1ccc2nnc(Cc3ccc4ncc(NC5CCOCC5)cc4c3)n2n1]

The title compound was prepared using the same procedure as described in the synthesis of Example 15 by starting from [3-(tetrahydro-pyran-4-ylamino)-quinolin-6-yl]-acetic acid methyl ester (Intermediate C4). ¹H-NMR (400 MHz, DMSO-d₆) δ ppm 1.40 (dd, 2H), 1.93 (d, 2H), 2.21 (s, 3H), 3.44 (dd, 2H), 3.45-3.60 (m, 1H), 3.88 (d, 2H), 4.63 (s, 2H), 6.18 (d, 1H), 7.03 (s, 1H), 7.32 (d, 1H), 5.55 (s, 1H), 7.68 (d, 1H), 7.73 (d, 1H), 8.24 (d, 1H), 8.40 (s, 1H), 12.26 (s, 1H). LCMS (method A): [M+H]⁺=418, t_{R}=2.03 min.

The activity of a compound according to the present invention can be assessed by the following in vitro & in vivo methods.

### 1. C-Met Enzyme Assay

The exemplified compounds of the present invention were assayed in an antibody based kinase phosphorylation assay as follows.

EPK c-MET Profiling Assay:

The EPK kinase assay for c-MET receptor tyrosine kinase was developed, using the purified recombinant GST-fusion protein, containing the cytoplasmic domain of the enzyme. GST-c-MET(969-1390) was purified by affinity chromatography.

The kinase assay is based on the LanthaScreen™ technology. LanthaScreen™ is the detection of Time-Resolved Fluorescence Resonance Energy Transfer (TR-FRET) using lanthanide chelates to measure interactions between various binding partners. In a TR-FRET kinase assay, a long-lifetime lanthanide donor species is conjugated to an antibody that specifically binds to a phosphorylated product of a kinase reaction that is labeled with a suitable acceptor fluorophore. This antibody-mediated interaction brings the lanthanide donor and the acceptor into proximity such that resonance energy transfer can take place, resulting in a detectable increase in the FRET signal.

The kinase reactions were performed in 384 well microtiter plates in a total reaction volume of 10.05 μL. The assay plates were prepared with 0.05 μL per well of test compound in the appropriate test concentration, as described under “preparation of compound dilutions”. The reactions were started by combining 5 μL of ATP solution with 5 μL of enzyme-substrate mix (consisting of kinase and substrate). The final concentrations in the kinase reactions were 25 mM Tris/HCl, 1 mM DTT, 0.025% Tween20, 10 μM sodium orthovanadate, 0.25% BSA, 0.5% DMSO, 10 mM MgCl₂, 3 mM MnCl₂, 2 μM ATP, 50 nM Fluorescein-PolyEAY, and 0.3 nM enzyme. The reactions were incubated for 60 minutes at room temperature and stopped by adding 5 μL of stop buffer (50 mM EDTA, 0.04% NP40, 20 mM Tris/HCl). Subsequently 5 μL of detection mix (50 mM Tris/HCl, 2 mM DTT, 0.05% Tween20, 20 μM sodium orthovanadate, 1% BSA, 1 nM Tb-PY20 antibody) were added to the stopped reactions. After 45 minutes incubation in dark at room temperature, the plates were measured in a Perkinelmer Envision fluorescence reader. The effect of compound on the enzymatic activity was in all assays obtained from the linear progress curves and normally determined from one reading (end point measurement). Results are summarized in the Table 1 below.

These endpoint results should therefore only be seen as an indicator for the activity range, since repeated measurements can result in about two times higher or lower values. Accordingly, “active” compounds of the invention have an IC₅₀ in this enzyme assay of less than 5000 nM, preferably less than 1000 nM, more preferably less than 200 nM and most preferably less than 10 nM.

TABLE 1
c-Met Inhibitory activity of compounds of the invention

| | c-Met Biochem |
|---|---|
| Example No. | IC₅₀ [nM] |
| 1 | 5 |
| 2 | 6 |
| 3-(S) | 5 |
| 3-(R) | 26 |
| 4 | 25 |
| 4-(S) | 23 |
| 4-(R) | 144 |
| 5 | 5 |
| 6 | 4 |
| 7 | 11 |
| 8 | 3 |
| 9 | 86 |
| 10 | 4 |
| 11 | 1 |
| 12 | 0.5 |
| 13 | 2 |
| 14 | 3 |
| 15 | 7 |
| 16 | 25 |
| 17 | 3 |
| 18 | 207 |
| 19 | 5 |
| 20 | 4 |
| 21-(S) | 3 |
| 21-(R) | 47 |
| 22 | 1 |
| 23 | 8 |
| 24-(S) | 5 |
| 24-(R) | 146 |
| 25 | 16 |
| 26 | 15 |
| 27 | 31 |
| 28 | 5 |
| 29 | 0.7 |
| 30 | 0.9 |
| 31 | 3 |
| 32 | 4 |
| 33 | 0.9 |
| 34 | 2 |

As it can be seen, each of the exemplified compounds of the invention has an IC₅₀ value in this enzyme assay below 200 nM.

### 2. GTL16 Cell Viability Assay:

GTL16 cell line is derived from a gastric cancer patient. GTL16 expresses high level of c-Met receptor tyrosine kinase due to the gene amplification. The growth of GTL16 is highly dependent on c-Met kinase activity; hence it is used as a cell-based assay to monitor the cellular activity of the c-Met kinase inhibitors.

GTL16 cells were seeded in DMEM medium with 10% FBS and 1% Pene. & Strep. at 5000 cells/well/90 μL in 96 well plate and incubated overnight for attachment at 37° C. in 5% CO₂ incubator. 10-fold serials dilutions of compounds were added to the cell as 10 μL/well. The final assay volume was 100 μl/well. The assay plates were incubated at 37° C. in 5% CO₂ incubator for 24 hours. The viability of cells was measured using the CellTiter Glo (Cat# G7573 Promega) according to the protocol suggested by the vender. Briefly, the plates were cooled at room temperature for 10 mins and 100 μl of CellTiter Glo reagent was added into each well. Plates were shaken for 10 mins. The chemiluminescent light unit was read in Envision from Perkin Elmer. All the tests were run at triplicates. The IC₅₀ was calculated using Spotfire software.

Results are summarized in the Table 2 below. “Active” compounds of the invention have an IC₅₀ in this enzyme assay of less than 500 nM, preferably less than 100 nM, more preferably less than 20 nM and most preferably less than 10 nM.

TABLE 2
c-Met inhibitory activity of selected compounds of the invention

| | GTL-16 |
|---|---|
| | Proliferation |
| Example No. | IC₅₀ [nM] |
| 1 | 9 |
| 2 | 2 |
| 3-(S) | 1 |
| 3-(R) | 27 |
| 4-(S) | 9 |
| 4-(R) | 99 |
| 5 | 1 |
| 6 | 0.5 |
| 7 | 8 |
| 8 | 0.3 |
| 9 | 311 |
| 10 | 1 |
| 11 | 6 |
| 12 | 1 |
| 13 | 10 |
| 14 | 2 |
| 15 | 13 |
| 16 | 491 |
| 17 | 10 |
| 18 | 317 |
| 19 | 12 |
| 20 | 17 |
| 21-(S) | 7 |
| 21-(R) | 30 |
| 22 | 3 |
| 23 | 11 |
| 24-(S) | 80 |
| 24-(R) | 126 |
| 25 | 12 |
| 26 | 93 |
| 27 | 240 |
| 28 | 2 |
| 29 | 1 |
| 30 | 1 |
| 31 | 23 |
| 32 | 256 |
| 33 | 1 |
| 34 | 10 |

Each of the exemplified compounds has an IC₅₀ value in this enzyme assay below 500 nM.

3. hPDE3 Assay

Phosphodiesterase-3 (PDE3) is one of a family of phosphodiesterases responsible for the regulation of cyclic nucleotide second messengers. Human PDE3 has high affinity for both cAMP and cGMP and is distributed in a wide range of tissues and cell types. Inhibitors of hPDE3 are potentially useful as inotropic/vasodilator, antithrombotic and anti-inflammatory agents (Komas et al. 1996). Agents that inhibit PDE3 were originally investigated for the treatment of heart failure but have unwanted arrhythmic side effects (Dart R. C., Medical Toxicology, Edition 3, page 708; Lippincott 2004).

PDE3 assays to measure the inhibitory potential of compounds at this enzyme are well known to the person skilled in the art. For example, cAMP and cGMP levels can be measured by the use of the tritium containing compounds ³HcAMP and ³HcGMP as described in [Hansen, R. S., and Beavo, J. A., PNAS 1982; 79: 2788-92]. To screen a compound pool comprised of a large number of compounds, the microtiter plate-based scintillation proximity assay (SPA) as described in [Bardelle, C. et al. (1999) Anal. Biochem. 275: 148-155] can be applied. Alternatively, the phosphodiesterase activity of the recombinant protein can be assayed using a commercially available SPA kit (Amersham Pharmacia). Such an assay for PDE3 was e.g. described within Kima et al (2004) Bioorganic & Medicinal Chemistry Letters, Vol 14(9): 2099-2103. An alternative PDE3 assay for measuring the PDE3 inhibitory potential of c-Met inhibitors was disclosed in WO 2010/138673.

A possible isolation method for human PDE3 from human platelets is disclosed within Ito et al (1996) Cell Signal. 1996 December; 8(8):575-81.

Here, compounds of formula I were screened for their ability to inhibit human PDE3 in the assay based on Amersham Pharmacia Biotech's Phosphodiesterase (PDE) [³H]-adenosine 3′,5′ cyclic phosphate ([³H]cAMP) Scintillation Proximity Assay (SPA). The assay is based on the hydrolysis of [³H]cAMP, by human platelet PDE3, to [³H]5′-adenoside monophosphate (5′-AMP). The [³H]5′-AMP is specifically captured by yttrium silicate SPA beads in the presence of zinc sulphate. When [³H]5′-AMP binds to the beads, β-particles are emitted and excite, by their proximity, the fluorophore in the beads and hence produce light. Free [³H]cAMP in turn does not activate the scintillant, since the unbound radioactivity is released too distant from the scintillant, and hence does not produce light.

### Materials

Optiplate and TopSeal-S (Can berra Packard)
Human platelet PDE3 (partially purified from human platelets)—a titration curve of human platelet PDE3 activity was performed to optimise the concentration of hPDE3 required in the assay.
Yttrium silicate SPA beads and [³H]cAMP (Amersham)
Tris-Base, magnesium chloride, ethylenediaminetetraacetic acid (di-sodium salt), bovine serum albumin BSA and cAMP (Sigma)

Solutions and Buffers:
Assay buffer:
7.56 g Tris-Base was dissolved in approximately 800 mL distilled water and the pH adjusted to 7.5 with 1 M hydrochloric acid. 10.3 mL 1 M magnesium chloride and 4.25 mL 0.5 M EDTA were added. The solution was made to 1 L with distilled water and stored at 4° C. On day of use 18 mL of the above solution was removed and 2 mL 5 mg/ml BSA were added thereto.
Enzyme buffer: 10 mM Tris-HCl at pH 7.5, 1 mM EDTA
Yttrium silicate SPA beads: 1 vial was reconstituted in 28 mL distilled water and stored at 4° C.

### Assay

The assay was performed in a final volume of 100 μL per well of an Optiplate (Can berra Packard).

A 10 μL aliquot of the test compound dissolved in DMSO/distilled water was placed in a well of an Optiplate plate, followed by the addition of 80 μL ‘Assay mix’ (5.5 μL [³H]cAMP and 88 μL “cold” cAMP were diluted to 8.8 mL using assay buffer). The reaction was started by adding 10 μL hPDE3 (50 μL stock hPDE3 solution was diluted 50 fold to 2.5 mL using enzyme buffer). The plate was incubated at room temperature for 30 min, the reaction was then terminated by the addition of 50 μL Yttrium silicate SPA beads (pre-warmed to room temperature) to all wells. The plate incubated at room temperature for at least 15 min. The plate was sealed using TopSeal-S according to the manufacturer's instructions and counted using a Packard TopCount, each well being counted for 1 min. IC₅₀ values were determined using non-linear regression.

Results of some exemplary compounds are summarized in the Table 3 below. Compounds of the invention have preferably high IC₅₀ values in this enzyme assay, preferably more than 500 nM, preferably more than 1 μM, more preferably more than 10 μM and most preferably more than 30 μM.

TABLE 3
PDE3 Inhibitory activity of selected compounds of the invention

| | hPDE3 |
|---|---|
| Example No. | IC₅₀ [μM] |
| 3-(S) | 2 |
| 3-(R) | 11 |
| 4-(S) | 1.2 |
| 4-(R) | 10 |
| 11 | >30 |
| 12 | 9 |
| 13 | 14 |
| 14 | 25 |
| 15 | >30 |
| 21-(S) | 4 |
| 21-(R) | 7 |
| 22 | 21 |
| 24-(S) | 8 |
| 24-(R) | 6 |
| 25 | 3 |
| 26 | >30 |
| 28 | >30 |
| 29 | >30 |
| 30 | 17 |
| 31 | >30 |
| 32 | >30 |

As it can be seen, each of the exemplified compounds has an IC₅₀ value in this enzyme assay above 1 μM.

Certain preferred compounds of the invention have good exposure in vivo, and/or have a favourable solubility profile. Assays to measure bioavailability, pharmacokinetic profiles and solubility are well known in the art.

Certain preferred compounds of the invention produce metabolites in vivo which themselves have a favourable solubility profile, thereby avoiding or limiting undesirable effects in vivo.

Preferred compounds of the invention are metabolically stable, and/or produce metabolites that doe not have undesirable effects in the body. For example the metabolites formed do not interfere, or have limited interference, with normal renal function.

Those skilled in the art will recognize, or be able to ascertain using no more than routine experimentation, many equivalents to the specific embodiments and methods described herein. Such equivalents are intended to be encompassed by the scope of the following claims.

1. A compound of formula (I) 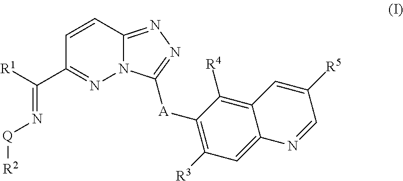
wherein
Q is O, NH or N(C₁-C₄)allyl,
A is a group selected from i or ii: 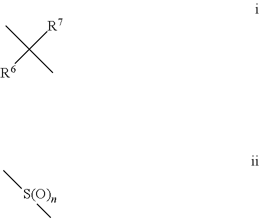
wherein
R⁶ is hydrogen, deuterium, OH, methyl or halo;
R⁷ is hydrogen, deuterium, halo, or (C₁-C₃)allyl, wherein said (C₁-C₃)alkyl is optionally substituted by one or more substituents independently selected from OH and halo;
or R⁶ and R⁷, together with the carbon to which they are attached form cyclopropyl, wherein said cyclopropyl is optionally substituted by methyl;
n is 0, 1 or 2;
R¹ is hydrogen, NH₂, or (C₁-C₄)alkyl, wherein said (C₁-C₄)allyl is optionally substituted by one or more substituents independently selected from OH, NH₃ and halo;
R² is
hydrogen,
(C₁-C₄)allyl, wherein said (C₁-C₄)alkyl is optionally substituted by one or more substituents independently selected from halo, hydroxy and methoxy, or
—(C₀-C₂)alkyl(C₃-C₆)cycloalkyl;
R³ and R⁴ are independently selected from hydrogen and halo;
R⁵ is
—(C₀-C₃)alkyl-heterocyclyl¹,
—(C₀-C₃)alkyl-(C₃-C₈)cycloalkyl,
—NR⁸R⁹, or
(C₁-C₃)allyl substituted by one or more OH or by —N((C₁-C₃)alkyl)₂,
wherein heterocyclyl¹ is a 4, 5, 6, 7 or 8 membered saturated, unsaturated or partially unsaturated mono- or bicyclic group comprising 1, 2 or 3 ring heteroatoms independently selected from N, O and S, wherein the total number of ring S atoms does not exceed 1, and the total number of ring O atoms does not exceed 1, and wherein heterocyclyl is optionally substituted by one or two substituents independently selected from —OH, —CONH₂, —N((C₁-C₃)alkyl)₂ and —NH₂,
R⁸ is hydrogen or (C₁-C₃)alkyl,
and R⁹ is (C₁-C₃)allyl, (C₃-C₈)cycloalkyl, or heterocyclyl², wherein heterocyclyl² is a 5 or 6-membered saturated or partially unsaturated monocyclic group comprising 1 or 2 ring heteroatoms independently selected from N, O and S, and optionally substituted by —OH or (C₁-C₃)allyl,
or a pharmaceutically acceptable salt thereof;
with the proviso that the compound is not (E)-1-{3-[3-(4-Methyl-piperazin-1-yl)-quinolin-6-ylmethyl]-[1,2,4]triazolo[4,3-b]pyridazin-6-yl}-ethanone O-(2-hydroxy-ethyl)-oxime.

2. A compound or pharmaceutically acceptable salt thereof as claimed in claim 1, wherein
R⁵ is
—(C₀-C₃)alkyl-heterocyclyl¹,
—(C₀-C₃)alkyl-(C₃-C₈)cycloalkyl, or
(C₁-C₃)allyl substituted by one or more OH,
wherein heterocyclyl¹ is a 4, 5, 6, 7 or 8 membered saturated, unsaturated or partially unsaturated mono- or bicyclic group comprising 1, 2 or 3 ring heteroatoms independently selected from N, O and S, wherein the total number of ring S atoms does not exceed 1, and the total number of ring O atoms does not exceed 1, and wherein heterocyclyl¹ is optionally substituted by one or two substituents independently selected from —OH, —NH₂, —N((C₁-C₃)alkyl)₂, —CONH₂, and (C₁-C₃)allyl.

3. A compound or pharmaceutically acceptable salt thereof as claimed in claim 1, wherein Q is —O— and R¹ is methyl.

4. A compound or pharmaceutically acceptable salt thereof as claimed in claim 1 wherein R² is hydrogen, cyclopropylmethyl-, ethyl, methyl or 2-hydroxyethyl.

5. A compound or pharmaceutically acceptable salt thereof as claimed in claim 4, wherein R² is hydrogen.

6. A compound or pharmaceutically acceptable salt thereof as claimed in claim 1, wherein A is 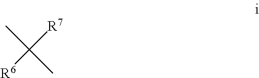
wherein R⁶ is hydrogen and R⁷ is hydrogen or methyl, or
R⁶ and R⁷, together with the carbon to which they are attached form cyclopropyl.

7. A compound or pharmaceutically acceptable salt thereof as claimed in claim 6, wherein R⁶ and R⁷ are both hydrogen.

8. A compound or pharmaceutically acceptable salt thereof as claimed in claim 1, wherein A is —S—.

9. A compound or pharmaceutically acceptable salt thereof as claimed in claim 1, wherein R³ and R⁴ are independently selected from hydrogen and fluoro.

10. A compound or pharmaceutically acceptable salt thereof as claimed in claim 1, wherein R⁵ is —NR⁸R⁹, wherein R⁸ is hydrogen or methyl, and R⁹ is cyclohexyl or heterocyclyl² optionally substituted by methyl.

11. A compound or pharmaceutically acceptable salt thereof as claimed in claim 10, wherein R⁵ is —NR⁸R⁹, wherein R⁸ is hydrogen and R⁹ is piperidin-4-yl or tetrahydropyran-4-yl, both optionally substituted by methyl.

12. A compound or pharmaceutically acceptable salt thereof as claimed in claim 1, wherein R⁵ is —(C₀-C₃)alkyl-heterocyclyl¹, or —(C₀-C₃)alkyl-(C₃-C₈)cycloalkyl, wherein heterocyclyl¹ is a 5, 6, 7 or 8 membered saturated, unsaturated or partially unsaturated mono- or bicyclic group comprising 1 or 2 ring heteroatoms independently selected from N, O and S, wherein the total number of ring S atoms does not exceed 1, and the total number of ring O atoms does not exceed 1, and wherein heterocyclyl¹ is optionally substituted by one or two (C₁-C₃)allyl groups or one —N((C₁-C₃)allyl)₂, —NH₂ or —OH group.

13. A compound or pharmaceutically acceptable salt thereof as claimed in claim 1, wherein R⁵ is —(C₁-C₃)alkyl-heterocyclyl¹, or —(C₀-C₃)alkyl-(C₃-C₈)cycloalkyl, wherein heterocyclyl¹ is a 5, 6, 7 or 8 membered saturated, unsaturated or partially unsaturated mono- or bicyclic group comprising 1 or 2 ring heteroatoms independently selected from N, O and S, wherein the total number of ring S atoms does not exceed 1, and the total number of ring O atoms does not exceed 1, and wherein heterocyclyl¹ is optionally substituted by one or two (C₁-C₃)allyl groups or one —N((C₁-C₃)allyl)₂, —NH₂ or —OH group.

14. A compound or pharmaceutically acceptable salt thereof as claimed in claim 12, wherein R⁵ is —(C₀-C₁)alkyl-heterocyclyl¹ or —(C₀-C₁)alkyl-(C₃-C₆)cycloalkyl, wherein heterocyclyl¹ is selected from tetrahydrofuranyl, tetrahydrothiophenyl, 3,6-dihydro-2H-pyridinyl, 1,2,3,4-tetrahydropyridinyl, 1,2,5,6-tetrahydropyridinyl, pyrrolidinyl, thiazolidinyl, morpholinyl, thiomorpholinyl, piperidinyl, piperazinyl, quinuclidinyl, 2,5-diaza-bicyclo[2.2.1]heptyl, pyrrolyl, furanyl, thiophenyl, pyrazolyl, imidazolyl, isoxazolyl, oxazolyl, oxazolinyl, oxazolidinyl, isothiazolyl, thiazolyl, pyridinyl, pyridazinyl, pyrimidinyl, pyrazinyl, 3,4-dihydro-2H-pyranyl, 5,6-dihydro-2H-pyranyl, 2H-pyranyl, tetrahydropyranyl, dihydro-1H-pyrrolyl, azepanyl, diazepanyl, oxazepanyl, and thiazepanyl, and wherein heterocyclyl¹ is optionally substituted by one or two methyl groups or one —N((C₁-C₃)alkyl)₂, —NH₂ or —OH group.

15. A compound or pharmaceutically acceptable salt thereof as claimed in claim 1, wherein R⁵ is —(C₀-C₁)alkyl-heterocyclyl¹, and heterocyclyl¹ is selected from 3,6-dihydro-2H-pyridin-1-yl, 1,2,3,4-tetrahydropyridin-1-yl, 1,2,5,6-tetrahydropyridin-1-yl, pyrrolidin-1-yl, thiazolidin-3-yl, morpholin-4-yl, thiomorpholin-4-yl, piperidin-1-yl, piperazin-1-yl, quinuclidin-1-yl, 2,5-diaza-bicyclo[2.2.1]hept-2-yl, pyrrol-1-yl, pyrazol-1-yl, imidazol-1-yl, H-isoxazol-2-yl, oxazol-3-yl, oxazolidin-3-yl, isothiazol-2-yl, thiazol-3-yl, pyridin-1-yl, pyridazin-1-yl, pyrimidin-1-yl, pyrazin-1-yl, dihydro-pyrrol-1-yl, azepan-1-yl, diazepan-1-yl, oxazepan-3-yl, and thiazepan-3-yl, and wherein heterocyclyl¹ is optionally substituted by one or two methyl groups or one —N((C₁-C₃)alkyl)₂, —NH₂ or —OH group.

16. A compound or pharmaceutically acceptable salt thereof as claimed in claim 15, wherein R⁵ is —CH₂-heterocyclyl¹.

17. A compound or pharmaceutically acceptable salt thereof as claimed in claim 16, wherein heterocyclyl¹ is not 4-methyl-piperazin-1-yl.

18. A compound or pharmaceutically acceptable salt thereof as claimed in claim 1, wherein R⁵ is selected from morpholin-4-ylmethyl, 4-methylpiperazin-1-ylmethyl, piperidin-1-ylmethyl, 1-methyl-1H-pyrazol-4-yl, morpholin-4-yl, 3,5-dimethyl-isoxazol-4-yl, (1S,4S)-5-methyl-2,5-diaza-bicyclo [2.2.1]hept-2-yl, 3-dimethylamino-pyrrolidin-1-yl and 4-hydroxypiperidin-1-yl.

19. A compound or pharmaceutically acceptable salt thereof as claimed in claim 1, wherein
Q is O or NH,
A is a group selected from i or ii′: 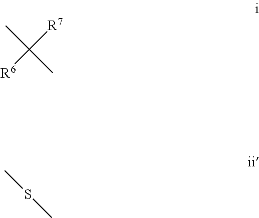
wherein
R⁶ is hydrogen;
R⁷ is hydrogen or methyl;
or R⁶ and R⁷, together with the carbon to which they are attached form cyclopropyl;
R¹ is methyl;
R² is
hydrogen,
(C₁-C₂)allyl, wherein said (C₁-C₂)alkyl is optionally substituted by hydroxy, or
—CH₂-cyclo(C₃-C₄)alkyl;
R³ and R⁴ are independently selected from hydrogen and fluoro;
R⁵ is
heterocyclyl¹,
—CH₂-heterocyclyl¹,
—(C₀-C₁)alkyl-(C₃-C₆)cycloalkyl,
—NR⁸R⁹, or
(C₁-C₃)allyl substituted by one or more OH or by —N((C₁-C₃)alkyl)₂,
wherein
heterocyclyl¹ is morpholin-4-yl, piperazin-1-yl, piperidin-1-yl, 1H-pyrazol-4-yl, isoxazol-4-yl, 2,5-diaza-bicyclo[2.2.1]hept-2-yl, pyrrolidin-1-yl, and wherein heterocyclyl¹ is optionally substituted by one or two methyl groups or one —N(CH₃)₂ or one —OH group,
R⁸ is hydrogen or (C₁-C₃)alkyl,
and R⁹ is (C₁-C₃)allyl, (C₃-C₆)cycloalkyl, or heterocyclyl², wherein heterocyclyl² is piperidin-4-yl or tetrahydropyran-4-yl, optionally substituted by methyl.

20. A compound or pharmaceutically acceptable salt thereof as claimed in claim 2, wherein
Q is —O—,
R¹ is methyl,
R² is hydrogen,
A is —CH₂— or —S—,
R³ and R⁴ are independently selected from hydrogen and fluoro,
R⁵ is —(C₀-C₁)alkyl-heterocyclyl¹, wherein heterocyclyl¹ is selected from morpholinyl, piperidinyl, piperazinyl, pyrazolyl, isoxazolyl, 2,5-diaza-bicyclo[2.2.1]heptyl, and pyrrolidinyl, and wherein heterocyclyl¹ is optionally substituted by one or two methyl groups or one —N(CH₃)₂ or one —OH group.

21. A compound or pharmaceutically acceptable salt thereof as claimed in claim 1, wherein the compound is selected from
No. 1 1-(3-{1-[3-(1-Methyl-1H-pyrazol-4-yl)quinolin-6-yl]-ethyl}-[1,2,4]triazolo[4,3-b]pyridazin-6-yl)-ethanone oxime
No. 2 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-ethyl-oxime
No. 3 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-methyl-oxime
No. 4 (E)-1-(3-{1-[3-(1-Methyl-1H-pyrazol-4-yl)quinolin-6-yl]-ethyl}-[1,2,4]triazolo[4,3-b]pyridazin-6-yl)-ethanone O-cyclopropylmethyl-oxime
No. 5 (E)-1-(3-{1-[3-(1-Methyl-1H-pyrazol-4-yl)quinolin-6-yl]-ethyl}-[1,2,4]triazolo[4,3-b]pyridazin-6-yl)-ethylidene]-hydrazine
No. 6 (E)-1-{3-[3-(1-Methyl-1H-pyrazol-4-yl)quinolin-6-ylmethyl]-[1,2,4]triazolo[4,3-b]pyridazin-6-yl}-ethanone O-methyl-oxime
No. 7 (E)-1-{3-[3-(1-Methyl-1H-pyrazol-4-yl)quinolin-6-ylmethyl]-[1,2,4]triazolo[4,3-b]pyridazin-6-yl}-ethanone O-cyclopropylmethyl-oxime
No. 8 (E)-1-{3-[3-(1-Methyl-1H-pyrazol-4-yl)quinolin-6-ylmethyl]-[1,2,4]triazolo[4,3-b]pyridazin-6-yl}-ethanone O-ethyl-oxime
No. 9 (E)-1-{3-[1-(3-(Morpholin-4-yl-methyl)quinolin-6-yl)-ethyl]-[1,2,4]triazolo[4,3-b]pyridazin-6-yl}-ethanone O-cyclopropylmethyl-oxime
No. 10 (E)-1-[3-(3-(Morpholin-4-yl)quinolin-6-ylsulfanyl)-[1,2,4]triazolo[4,3-b]pyridazin-6-yl]-ethanone oxime
No. 11 (E)-1-[3-((5,7-Difluoro-3-morpholin-4-yl-quinolin-6-yl)methyl)-[1,2,4]triazolo[4,3-b]pyridazin-6-yl]-ethanone oxime
No. 12 (E)-1-(3-((3-(1-Methyl-1H-pyrazol-4-yl)quinolin-6-yl)methyl)-[1,2,4]triazolo[4,3-b]pyridazin-6-yl)-ethanone oxime
No. 13 (E)-1-(3-((3-Morpholin-4-yl-quinolin-6-yl)methyl)-[1,2,4]triazolo[4,3-b]pyridazin-6-yl)-ethanone oxime
No. 14 (E)-1-(3 #3-(4-Methylpiperazin-1-yl)quinolin-6-yl)methyl)-[1,2,4]triazolo[4,3-b]pyridazin-6-yl)-ethanone oxime
No. 15 (E)-1-(3-((3-Morpholin-4-yl-methyl-quinolin-6-yl)methyl)-[1,2,4]triazolo[4,3-b]pyridazin-6-yl)-ethanone oxime
No. 16 (E)-1-(3-((3-(4-Methylpiperazin-1-yl-methyl)quinolin-6-yl)methyl)-[1,2,4]triazolo[4,3-b]pyridazin-6-yl)-ethanone oxime
No. 17 (E)-1-(3-((5,7-Difluoro-3-((morpholin-4-yl)-methyl)quinolin-6-yl)methyl)-[1,2,4]triazolo[4,3-b]pyridazin-6-yl)-ethanone oxime
No. 18 (E)-1-(3-((3-(Piperidin-1-ylmethyl)quinolin-6-yl)methyl)-[1,2,4]triazolo[4,3-b]pyridazin-6-yl)-ethanone oxime
No. 19 (E)-1-(3-((3-((1S,4S)-5-Methyl-2,5-diazabicyclo[2.2.1]heptan-2-yl)quinolin-6-yl)methyl)-[1,2,4]triazolo[4,3-b]pyridazin-6-yl)-ethanone oxime
No. 20 (E)-1-(3-((3-(4-Hydroxypiperidin-1-yl)quinolin-6-yl)methyl)-[1,2,4]triazolo[4,3-b]pyridazin-6-yl)-ethanone oxime
No. 21 (E)-1-(3-(1-(5,7-Difluoro-3-(1-methyl-1H-pyrazol-4-yl)quinolin-6-yl)ethyl)-[1,2,4]triazolo[4,3-b]pyridazin-6-yl)-ethanone oxime
No. 22 (E)-1-(3-((5,7-Difluoro-3-(1-methyl-1H-pyrazol-4-yl)quinolin-6-yl)methyl)-[1,2,4]triazolo[4,3-b]pyridazin-6-yl)-ethanone oxime
No. 23 (E)-1-(3-((3-(3,5-Dimethylisoxazol-4-yl)-5,7-difluoroquinolin-6-yl)methyl)-[1,2,4]triazolo[4,3-b]pyridazin-6-yl)-ethanone oxime
No. 24 (E)-1-(3-(1-(5,7-Difluoro-3-(2-hydroxypropan-2-yl)quinolin-6-yl)ethyl)-[1,2,4]triazolo[4,3-b]pyridazin-6-yl)-ethanone oxime
No. 25 (E)-1-(3-((3-Cyclohexyl-5,7-difluoroquinolin-6-yl)methyl)-[1,2,4]triazolo[4,3-b]pyridazin-6-yl)-ethanone O-2-hydroxyethyl oxime
No. 26 (E)-1-(3-((3-Cyclohexyl-5,7-difluoroquinolin-6-yl)methyl)-[1,2,4]triazolo[4,3-b]pyridazin-6-yl)-ethanone oxime,
No. 27 (E)-1-(3-({1-[3-(4-Methyl-piperazin-1-yl)quinolin-6-yl]-cyclopropyl}-[1,2,4]triazolo[4,3-b]pyridazin-6-yl)-ethanone oxime,
No. 28 (E)-1-(3-((3-(4-Methylpiperazin-1-yl)quinolin-6-yl)sulfanyl)-[1,2,4]triazolo[4,3-b]pyridazin-6-yl)-ethanone oxime
No. 29 (E)-1-(3-((3-(4-Hydroxypiperidin-1-yl)quinolin-6-yl)sulfanyl)-[1,2,4]triazolo[4,3-b]pyridazin-6-yl)-ethanone oxime
No. 30 (E)-1-(3-((3-((Tetrahydro-2H-pyran-4-yl)amino)quinolin-6-yl)sulfanyl)-[1,2,4]triazolo[4,3-b]pyridazin-6-yl)-ethanone oxime
No. 31 (E)-1-(3-((3-((Morpholin-4-yl)-methyl) quinolin-6-yl)sulfanyl)-[1,2,4]triazolo[4,3-b]pyridazin-6-yl)-ethanone oxime
No. 32 (E)-1-(3-((3-((Diethylamino)methyl)quinolin-6-yl)sulfanyl)-[1,2,4]triazolo[4,3-b]pyridazin-6-yl)ethanone oxime,
No. 33 (E)-1-(3-((3-(3-(Dimethylamino)pyrrolidin-1-yl)quinolin-6-yl)sulfanyl)-[1,2,4]triazolo[4,3-b]pyridazin-6-yl)ethanone oxime, and
No. 34 (E)-1-{3-[3-(Tetrahydro-pyran-4-ylamino)-quinolin-6-ylmethyl]-[1,2,4]triazolo[4,3-b]pyridazin-6-yl}-ethanone oxime.

22-24. (canceled)

25. A pharmaceutical composition comprising a compound of formula (I) as claimed in claim 1, or a pharmaceutically acceptable salt thereof, and at least one pharmaceutically acceptable carrier and/or diluents and optionally one or more further therapeutic agents.

26. A compound of formula (I) or a pharmaceutically acceptable salt thereof, as claimed in claim 1, in combination with one or more additional therapeutically active agents.

27. (canceled)

28. A method of treating a c-Met related disorder or condition which involves administering to a subject in need thereof an effective amount of a compound of formula (I) or a pharmaceutically acceptable salt thereof as claimed in claim 1.
